(19) United States
(12) Patent Application Publication
(10) Pub. No.: US 2023/0330562 A1
(43) Pub. Date: Oct. 19, 2023

(54) METHODS OF PREPARING PROTEIN-OLIGONUCLEOTIDE COMPLEXES

(71) Applicant: Dyne Therapeutics, Inc., Waltham, MA (US)

(72) Inventors: Timothy Weeden, Waltham, MA (US); Scott Hilderbrand, Waltham, MA (US); Sean Spring, Waltham, MA (US); Peiyi Shen, Waltham, MA (US); Benjamin Vieira, Waltham, MA (US)

(73) Assignee: Dyne Therapeutics, Inc., Waltham, MA (US)

(21) Appl. No.: 18/024,486

(22) PCT Filed: Sep. 3, 2021
(86) PCT No.: PCT/US21/49141

§ 371 Date: Mar. 2, 2023

Related U.S. Application Data
(60) Provisional application No. 63/074,439, filed on Sep. 3, 2020.
(60) Provisional application No. 63/074,436, filed on Sep. 3, 2020.

(51) Int. Cl. B01D 15/38; A61K 47/68

(52) U.S. Cl. CPC B01D 15/3847; A61K 47/6807; A61K 47/6849

(57) ABSTRACT

Aspects of the disclosure relate to methods of purifying complexes comprising a protein (e.g., antibody) covalently linked to a molecular payload (e.g., a charge-neutral oligonucleotide, a charged oligonucleotide, or a hydrophobic small molecule) using mixed-mode resin that comprises positively-charged metal sites and negatively charged ionic sites, e.g., hydroxyapatite resin. Methods of producing the complexes are also provided.

### RELATED APPLICATIONS

This application claims priority under 35 U.S.C. § 119(e) to U.S. Provisional Application Ser. No. 63/074,439, entitled “METHODS OF PREPARING PROTEIN-OLIGONUCLEOTIDE COMPLEXES”, filed on Sep. 3, 2020, and to U.S. Provisional Application Ser. No. 63/074,436, entitled “METHODS OF PREPARING PROTEIN-OLIGONUCLEOTIDE COMPLEXES”, filed on Sep. 3, 2020; the contents of each of which are incorporated herein by reference in their entirety.

### FIELD OF THE INVENTION

The present application relates to methods of purifying complexes (e.g., protein-oligonucleotide conjugates and antibody-drug conjugates).

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Sep. 3, 2021, is named D082470043WO00-SEQ-ZJG and is 57,060 bytes in size.

### BACKGROUND

In recent years, several oligonucleotides (e.g., antisense oligonucleotides) have been developed to combat tissue- or cell-specific diseases (e.g., muscle-specific diseases, e.g., various forms of muscular dystrophy). It has, nonetheless, proven challenging to effectively deliver these oligonucleotides to their desired tissues or cells.

### SUMMARY

Complexes comprising tissue- or cell-specific proteins (e.g., antibodies) covalently linked to therapeutic oligonucleotides offer excellent opportunities for delivery of said therapeutic oligonucleotides. Such therapeutic oligonucleotides include, for example, charge-neutral oligonucleotides (e.g., PMOs, PNAs, and others) as well as charged oligonucleotides (e.g., gapmers, mixers, siRNAs, and others). However, purification and isolation of said complexes away from excess protein and oligonucleotide presents challenges. The present disclosure, in some aspects, provide methods of processing complexes comprising protein covalently linked to oligonucleotides that separate out unconjugated oligonucleotides and proteins (e.g., antibodies) from the complexes. Methods of producing the complexes are also provided. In some embodiments, the methods of producing the complexes described herein reduces the level of unlinked antibodies that comprises an alkyne group. In some embodiments, the methods of producing the complexes described herein results in only trace amounts of unlinked antibodies that comprises an alkyne group after the conjugation reaction.

One aspect of the present disclosure relates to a method of processing complexes each comprising an antibody covalently linked to one or more charge-neutral oligonucleotides, the method comprising:

(i) contacting a mixture comprising an organic solvent, the complexes and unlinked charge-neutral oligonucleotides with a mixed-mode resin that comprises positively-charged metal sites and negatively charged ionic sites, under conditions in which the complexes adsorb to the mixed-mode resin, and
(ii) eluting the complexes from the mixed-mode resin under conditions in which the complexes dissociate from the mixed-mode resin. In some embodiments, the organic solvent is Dimethylacetamide (DMA), isopropyl alcohol (IPA), dimethyl sulfoxide (DMSO), acetonitrile (ACN), or propylene glycol (PG). In some embodiments, the organic solvent is at 5%-30% (v/v) in the mixture in step (i), optionally wherein the organic solvent is at 15% (v/v) in the mixture in step (i). In some embodiments, the organic solvent is at 30% (v/v) in the mixture in step (i).

In some embodiments, the mixture in step (i) further comprises up to 10 mM phosphate ions and/or up to 20 mM chloride ions. In some embodiments, the method further comprises washing the mixed-mode resin between step (i) and step (ii) with a washing solution comprising of an organic solvent, optionally wherein the organic solvent is Dimethylacetamide (DMA), isopropyl alcohol (IPA), dimethyl sulfoxide (DMSO), acetonitrile (ACN), or propylene glycol (PG). In some embodiments, the organic solvent is at 5%-30% (v/v) in the washing solution, optionally wherein the organic solvent is at 15% (v/v) in the washing solution. In some embodiments, the organic solvent is at 30% (v/v) in the washing solution. In some embodiments, the washing solution further comprises up to 10 mM phosphate ions and/or up to 20 mM chloride ions.

In some embodiments, step (ii) comprises applying an elution solution to the mixed-mode resin to elute the complexes, wherein the elution solution comprises an organic solvent, optionally wherein the organic solvent is Dimethylacetamide (DMA), isopropyl alcohol (IPA), dimethyl sulfoxide (DMSO), acetonitrile (ACN), or propylene glycol (PG). In some embodiments, the organic solvent is at 10%-30% (v/v) in the elution solution, optionally wherein the organic solvent is at 10% (v/v) in the elution solution. In some embodiments, the elution solution comprises at least 30 mM phosphate ions, optionally wherein the elution solution comprises at least 100 mM phosphate ions. In some embodiments, the elution solution comprises a gradually increasing concentration of phosphate ions, optionally wherein the concentration of the phosphate ions increases from at least 10 mM to at least 100 mM. In some embodiments, the elution solution has a pH of 7.6-8.5.

Another aspect of the present disclosure relates to a method of producing a complex comprising an antibody covalently linked to one or more oligonucleotides, the method comprising:

(i) obtaining an oligonucleotide comprising a structure of:

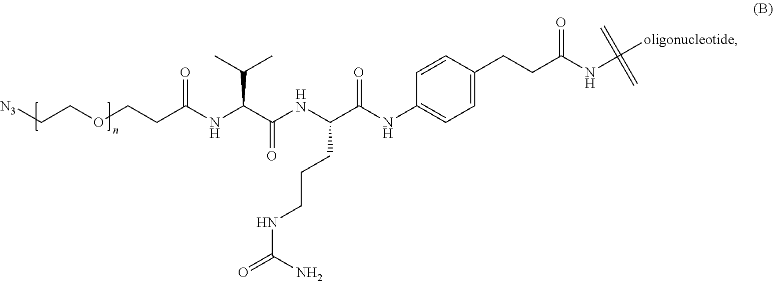

wherein n is 3;
(ii) obtaining an antibody comprises a structure of:

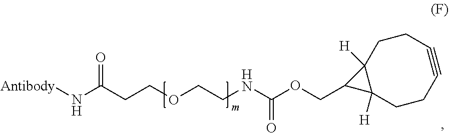

wherein m is 4; and
(iii) reacting the oligonucleotide in step (i) and the antibody obtained in step (ii) to obtain the complex.

Another aspect of the present disclosure relates to a method of producing a complex comprising an antibody covalently linked to one or more oligonucleotides, the method comprising:

(i) obtaining an oligonucleotide comprising a structure of:

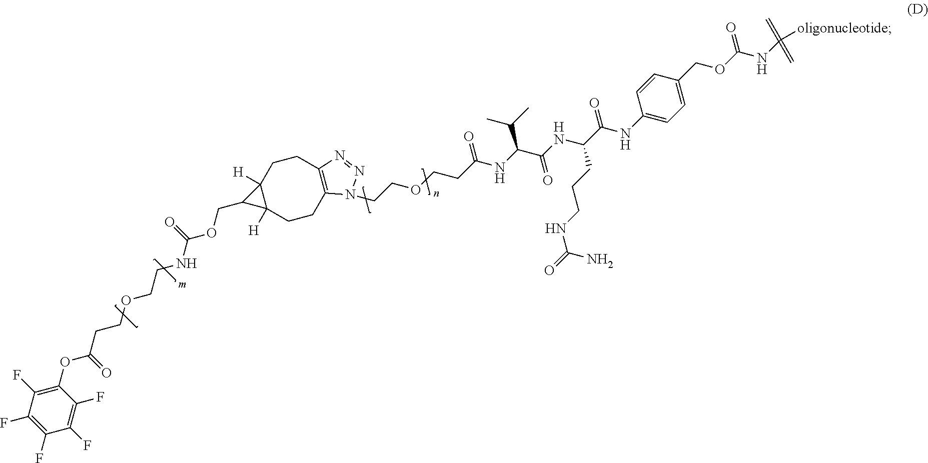

wherein n is 3 and wherein m is 4;
(ii) obtaining an antibody; and
(iii) reacting the oligonucleotide in step (i) and the antibody obtained in step (ii) to obtain the complex.

In some embodiments, the complex comprises a structure of:

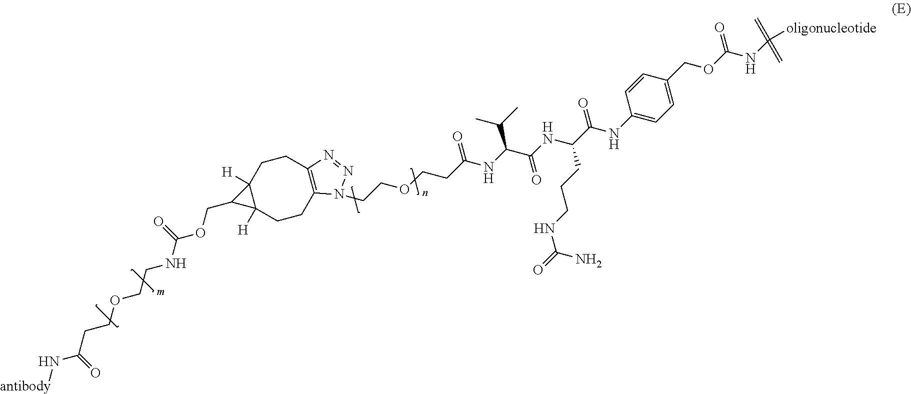

wherein n is 3 and m is 4, and wherein the antibody is linked via a lysine.

Another aspect of the present disclosure relates to a mixture comprising complexes, each complex comprising an antibody covalently linked to one or more oligonucleotides, and unlinked oligonucleotides, wherein the mixture is produced by a method comprising:

(i) obtaining a first intermediate comprising an oligonucleotide covalently linked to a cleavable linker comprising a valine-citrulline sequence;
(ii) linking the first intermediate obtained in step (i) with a compound comprising a bicyclononyne to obtain a second intermediate; and
(iii) linking the second intermediate obtained in step (ii) to an antibody to obtain the complexes;
wherein the compound comprising the bicyclononyne is present in the reaction of step (iii) in an amount that is less than 5% of the starting amount of the compound in step (ii), optionally wherein the oligonucleotide is covalently linked the cleavable linker comprising the valine-citrulline sequence at the 5′ end and/or the antibody is linked via a lysine.

Another aspect of the present disclosure relates to a mixture comprising complexes, each complex comprising an antibody covalently linked to one or more oligonucleotides, and ii) unlinked oligonucleotides, wherein the mixture is produced by a method comprising:

(i) combining one or more oligonucleotides with a linker of Formula (A):

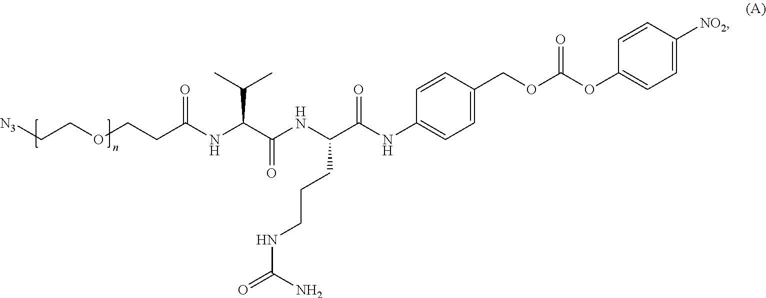

wherein n is 3; under reaction conditions that produce product of Formula (B):

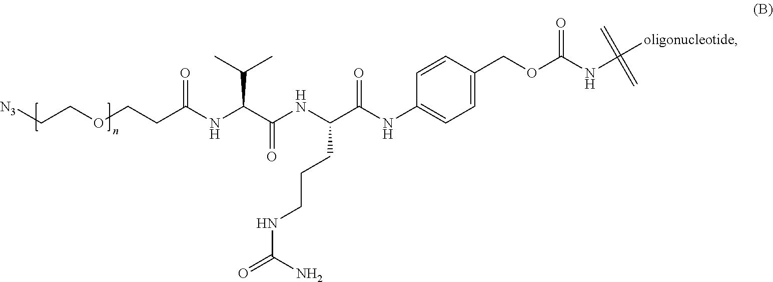

wherein n is 3;
(ii) contacting the product of Formula (B) with a compound of Formula (C):

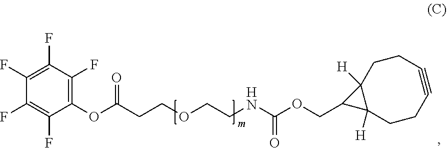

wherein m is 4; under reaction conditions that produce a product of Formula (D):

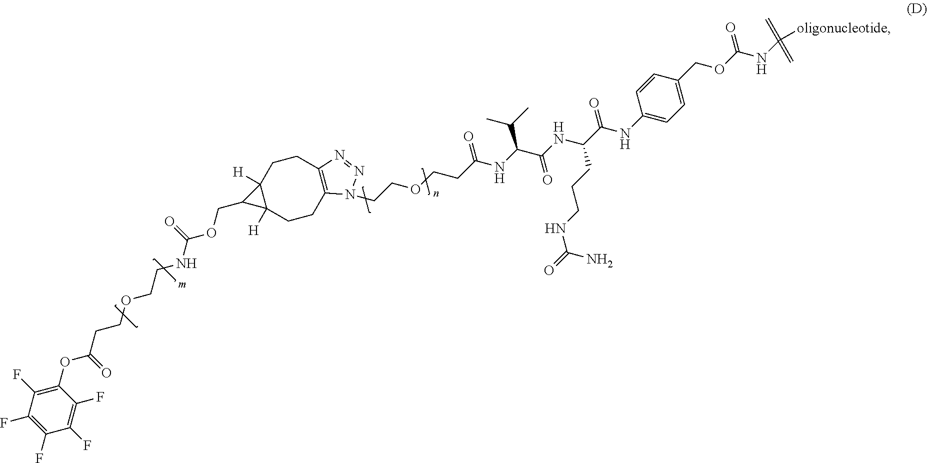

wherein n is 3 and m is 4; and
(iii) contacting the product of Formula (D) with an antibody under reaction conditions that produce a complex of Formula (E):

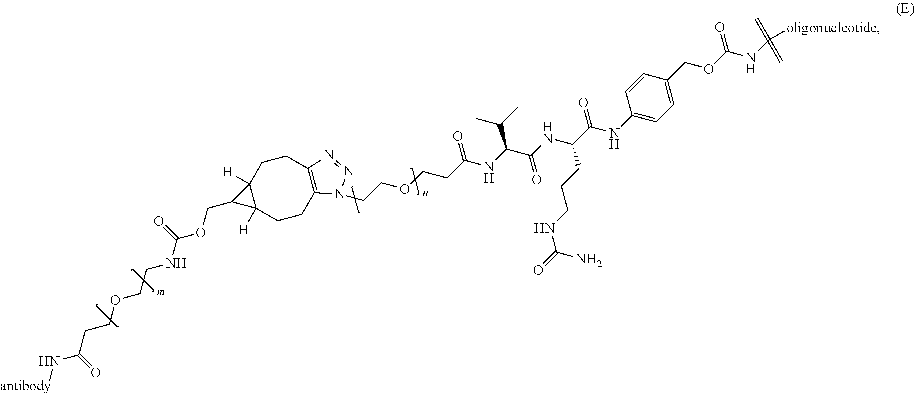

wherein n is 3 and m is 4;
wherein the compound of Formula (C) is present in the reaction of step (iii) in an amount that is less than 5% of the starting amount of the compound of Formula (C) in the reaction of step (ii).

Another aspect of the present disclosure relates to a method of processing complexes each comprising an antibody covalently linked to one or more oligonucleotides, the method comprising:

(i) contacting the mixture of either one of the two mixtures described above with a mixed-mode resin that comprises positively-charged metal sites and negatively charged ionic sites, under conditions in which the complexes adsorb to the mixed-mode resin, wherein the mixture comprises trace amounts of unlinked antibodies that comprise an alkyne group; and
(ii) eluting the complexes from the mixed-mode resin under conditions in which the complexes dissociate from the mixed-mode resin. In some embodiments, the mixture in step (i) has not been subjected to previous purification. In some embodiments, the mixture of step (i) comprises trace amounts of phosphate ions and/or chloride ions. In some embodiments, the method further comprises washing the mixed-mode resin between step (i) and step (ii) with a washing solution comprising up to 20 mM phosphate ions and/or up to 30 mM chloride ions, optionally wherein the solution comprises up to 10 mM phosphate ions and/or up to 25 mM chloride ions. In some embodiments, the washing solution has a pH of 5.0-7.6. In some embodiments, most or all unlinked oligonucleotides are removed from the mixed-mode resin in the washing step.

In some embodiments, step (ii) comprises applying an elution solution comprising at least 30 mM phosphate ions and/or at least 50 mM chloride ions to the mixed-mode resin to elute the complexes, optionally wherein the elution solution comprises at least 100 mM phosphate ions and/or at least 100 mM chloride ions. In some embodiments, the elution solution has a pH of 7.5-8.5.

In some embodiments, the antibody is an anti-transferrin receptor antibody. In some embodiments, the oligonucleotide is a charged oligonucleotide. In some embodiments, the oligonucleotide is a negatively charged oligonucleotide. In some embodiments, the oligonucleotide is single-stranded. In some embodiments, the oligonucleotide is an antisense oligonucleotide, optionally a gapmer. In some embodiments, the oligonucleotide is one strand of a double stranded oligonucleotide, optionally wherein the double stranded oligonucleotide is an siRNA, and optionally wherein the one strand is the sense strand of the siRNA. In some embodiments, the oligonucleotide comprises at least one modified internucleotide linkage, optionally wherein the at least one modified internucleotide linkage is a phosphorothioate linkage. In some embodiments, the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.

### BRIEF DESCRIPTION OF THE DRAWINGS

FIGS. 1A-1C show the chemical structures of molecules involved in the linking of an anti-TfR antibody to an oligonucleotide payload. FIG. 1A shows the structure of oligonucleotide-PAB-VC-PEG3-azide. FIG. 1B shows endo-BCN-PEG4-PFP ester. FIG. 1C shows oligonucleotide-PAB-VC-PEG3-triazole-PEG4-PFP ester. BCN: Bicyclononyne. PFP: Pentafluorophenyl. PAB: 4-aminobcnzoic acid. VC: val-cit. In all FIGS. 1A-1C, n is 3 and m is 4.

FIG. 2 shows an SDS-PAGE gel of crude Fab-oligonucleotide conjugates produced in reactions containing various ratios of anti-TfR Fab to oligonucleotide-PAB-VC-PEG3-triazole-PEG4-PFP ester. The leftmost lane shows a molecular weight ladder and the second lane shows unreacted anti-TfR Fab. Lanes labeled A, B, C, D, E and F show reaction products. Labels D0, D1, D2 and D3 indicate reaction products having drug to antibody ratios of 0, 1, 2 and 3, respectively.

FIG. 3 shows an SDS-PAGE gel of crude Fab-oligonucleotide conjugates produced in reactions containing various ratios of anti-TfR Fab to oligonucleotide-PAB-VC-PEG3-triazole-PEG4-PFP ester and various solvent conditions. The leftmost lane shows a molecular weight ladder. Lanes labeled G, H, T, J, K, L and M show reaction products.

FIG. 4 shows results of RP C18 UPLC monitoring of the reaction between endo-BCN-PEG4-PFP ester and oligonucleotide-PAB-VC-PEG4-azide over time. The peak area of the endo-BCN-PEG4-PFP ester starting material was measured over time at 220 nm and is shown as a percent of the original starting material peak at time 0.

FIG. 5 shows a size exclusion chromatography (SEC) chromatogram of anti-TfR Fab′-oligonucleotide conjugate after purification, measured at 260 nm and 280 nm.

FIG. 6 shows an SDS-PAGE gel anti-TfR Fab-oligonucleotide conjugate after purification. The rightmost lane shows a molecular weight ladder. The remaining three lanes show fractions eluted from a purification column. Labels D0, D1, D2 and D3 indicate reaction products having drug to antibody ratios of 0, 1, 2 and 3, respectively.

FIG. 7 is a graph showing the activities of the complexes processed using the methods described herein in reducing DMPK mRNA level in vitro. Complex 1 was prepared via 2-step conjugation: see Example 6. Complex 2 was prepared via pre-reaction conjugation: see Example 1.

FIG. 8 shows analytical HPLC-SEC traces of crude and purified anti-TfR-BCN reaction product. The curve for the crude product shows a second major peak at ˜17.4 minutes which is absent from the curve for the purified product, which demonstrates removal of the unconjugated BCN. BCN: bicyclononyne.

FIG. 9 shows analytical RP-HPLC of crude linker-oligonucleotide reaction product, demonstrating peaks corresponding to free oligonucleotide as well as free linker in addition to the peak for the desired oligonucleotide-linker product.

FIG. 10 shows analytical RP-HPLC of linker-oligonucleotide reaction product purified by alcohol precipitation. The curve shows substantially reduced free oligonucleotide peak and almost eliminated free linker peak relative to the curve shown in FIG. 9, indicating removal of the free oligonucleotide and linker by the purification process.

FIG. 11 shows LCMS of purified oligonucleotide-linker product, showing only one main peak.

FIG. 12 shows SDS-PAGE analysis of anti-TfR-oligonucleotide conjugation reaction product. The left lane shows a molecular weight ladder and the right lane shows the reaction product. DAR0, DAR1, DAR2, DAR3 and DAR4 labels indicate products with a drug-antibody ratio (DAR) of 0, 1, 2, 3 or 4, respectively.

FIG. 13 shows analytical SEC curves for anti-TfR-oligonucleotide conjugation reaction product.

FIG. 14 shows RP-C18 UPLC monitoring of pre-reaction progress by disappearance of endo-BCN-PEG4-PFP ester starting material over time at 220 nm at 5, 35, and 65 minutes reaction duration. PFP: Pentafluorophenyl.

FIG. 15 shows SDS-PAGE analysis of crude reaction mixture after conjugation of anti-TfR Fab with oligonucleotide-PAB-VC-PEG3-triazole-PEG4-PFP ester proceeded for 20 hours. The left lane shows a molecular weight ladder. D0, D1, D2, D3, D4, D5 and D6 labels indicate products with a drug-antibody ratio (DAR) of 0, 1, 2, 3, 4, 5 or 6, respectively.

FIG. 16 shows a size exclusion chromatography (SEC) chromatogram (260 nm) of the HA flow-through fraction during chromatographic purification. The chromatogram shows free (unconjugated) payload species at 10.5 and 11.3 minutes. Little to no Fab-oligonucleotide conjugate (main peak at ˜9.1 min) was observed in the load flow-through.

FIG. 17 shows an SEC chromatogram (260 nm) of the HA elution peak during chromatographic purification. The chromatogram indicates complete removal of free (unconjugated) oligonucleotide payload species (expected peaks at ˜10.5 and ˜11.3 min), and instead shows only Fab-oligonucleotide conjugates (main peak at ˜9.1 min).

FIG. 18 shows overlaid SEC chromatograms (260 nm) of a 24 μg injection of crude conjugate reaction product (4.0 ul at 6 μg/μL; “Crude reaction mixture” curve with two major peaks) and a theoretical 24 μg of Fab-oligonucleotide conjugate in the HA eluate, assuming 100% recovery (7.4 μL at 3.24 μg/μL and a volume of 13.9 mL; “HA eluate” curve with only one major peak).

FIGS. 19A-19B show SEC chromatograms of the final purified anti-TfR-oligonucleotide Fab-oligonucleotide conjugate at 260 nm (FIG. 19A) and 280 nm (FIG. 19B).

FIG. 20 shows SDS-PAGE analysis of purified reaction product in 50 mM His (pH 6.0) buffer, after conjugation of anti-TfR Fab to oligonucleotide to form the Fab-oligonucleotide conjugate. The left lane shows the purified Fab-oligonucleotide conjugate reaction product, and the right lane shows a molecular weight ladder.

FIG. 21 shows SDS-PAGE analysis of crude reaction products after conjugation of oligonucleotide-PAB-VC-PEG3-triazole-PEG4-PFP ester to anti-TfR Fab at various molar ratios. The left lane shows a molecular weight ladder. Lanes A, B, C, and D show products of conjugation reactions conducted with 2, 4, 6, or 10 molar equivalents of BCN relative to anti-TfR, respectively. The right lane shows unreacted anti-TfR Fab. D0, D1, D2, D3, D4, D5 and D6 labels indicate products with a drug-antibody ratio (DAR) of 0, 1, 2, 3, 4, 5 or 6, respectively.

FIG. 22 shows SDS-PAGE analysis of crude reaction products after conjugation of oligonucleotide-PAB-VC-PEG3-triazole-PEG4-PFP ester to anti-TfR Fab at various molar ratios. The leftmost and rightmost lanes show a molecular weight ladder. Lanes I, H, G, F and E show products of conjugation reactions conducted with 8, 6, 5, 4, or molar equivalents of BCN relative to anti-TfR, respectively. D0, D1, D2, D3, D4, D5 and D6 labels indicate products with a drug-antibody ratio (DAR) of 0, 1, 2, 3, 4, 5 or 6, respectively.

FIG. 23 shows the hydrolysis rate of the endo-BCN-PEG4-PFP ester. The blue curve result shows that with 1:1 DMA, the endo-BCN-PEG4-PFP ester has an initial hydrolysis rate of about 0.9%/h. The green curve shows the reaction between a model val-cit-PAB-PEG3-azide payload with endo-BCN-PEG4-PFP ester under the same 1:1 DMA reaction conditions. Both curves show absorbance at 220 nm.

FIGS. 24A-24C show SEC-UPLC analysis of the crude reaction mixture (FIG. 24A), the sample flow through (FIG. 24B), and the pooled HA eluate (FIG. 24C). The crude reaction mixture comprises the free oligonucleotide and anti-TfR-oligonucleotide Fab-oligonucleotide conjugate. The sample flow through comprises high DAR species, species with high antibody-oligonucleotide conjugation. The pooled HA eluate comprises residual oligonucleotide and antibody-oligonucleotide conjugates.

FIG. 25 shows SEC-UPLC analysis of the buffer exchanged final conjugate. The overall conjugation efficiency yield is 62% with residual oligonucleotide appearing in the right peak.

FIGS. 26A-26B show SEC-UPLC analysis of the pooled HA eluate (FIG. 26A) and the sample flow through (FIG. 26B). The sample was maintained at 21° C., and a flow rate of 0.25 mL/min. The mobile phase comprises 100 mM sodium phosphate and 10% MeCN at pH 7.3. The pooled HA eluate and sample flow through show the presence of free oligonucleotide.

FIG. 27 shows SEC-UPLC of the final antibody fragment-drug conjugates (FDC). The sample was maintained at 21° C., and a flow rate of 0.25 mL/min. The mobile phase comprises 100 mM NaPO and 10% MeCN at pH 7.3. 47.2% of the conjugates were recovered with free oligonucleotides appearing in the right peak.

FIGS. 28A-28B show HA purification chromatogram (FIG. 28A) and SDS-PAGE for each solution (FIG. 28B). The reaction mixture pH was adjusted to 5.7 with 500 mM MES at pH 3.5.

FIGS. 29A-29C show sample flow through chromatograms by SEC with different equilibration buffers and at a loading ratio of 6 mg/mL. FIG. 29A shows the sample flow through in a 10 mM sodium phosphate equilibration buffer at pH 5.6 with 0% IPA. FIG. 29B shows the sample flow through in a 10 mM NaPO equilibration buffer at pH 5.6 with 25% IPA. FIG. 29C shows the sample flow through in a 5 mM sodium phosphate equilibration buffer at pH 5.6 with 25% IPA.

### DETAILED DESCRIPTION OF CERTAIN EMBODIMENTS

As described herein, the present disclosure provides methods of processing (e.g., production, purification) complexes, e.g., complexes comprising proteins (e.g., muscle-targeting agents (e.g., an antibody) covalently linked to molecular payloads (e.g., oligonucleotides or small molecules)). In some embodiments, the molecular payload in the complexes process by the methods described herein is an oligonucleotide (e.g., charge-neutral oligonucleotide (e.g., PMO) or charged oligonucleotide) or a hydrophobic small molecule. In some embodiments, a complex or plurality of complexes comprising a protein (e.g., an antibody) covalently linked to a molecular payload (e.g., a charge-neutral oligonucleotide or a charged oligonucleotide) is purified from a mixture comprising said complex and unlinked (e.g., excess) molecular payload (e.g., a charge-neutral oligonucleotide or a charged oligonucleotide) using a mixed-mode resin that comprises positively-charged metal sites and negatively charged ionic sites (e.g., hydroxyapatite resin, ceramic hydroxyapatite resin, hydroxyfluoroapatite resin, fluoroapatite resin, chlorapatite resin), wherein an organic solvent is present in the mobile phase of the mixed-mode resin chromatography. The presence of the organic solvent in the mobile phase of the mixed-mode chromatography reduces the non-specification interactions between molecular payloads such as oligonucleotides (e.g., charge-neutral oligonucleotides (e.g., PMO) or charged oligonucleotide) and hydrophobic small molecules with the mixed-mode resin and increases the yields of the complexes.

In some embodiments, the complexes being purified are particularly useful for delivering molecular payloads (e.g., oligonucleotides) that modulate expression or activity of target genes in muscle cells, e.g., in a subject having or suspected of having a muscle disease. For example, in some embodiments, complexes are useful for treating subjects having rare muscle diseases, including myotonic dystrophy (e.g., myotonic dystrophy type 1), Facioscapulohumeral muscular dystrophy (FSHD), Pompe disease, Centronuclear myopathy, Fibrodysplasia Ossificans Progressiva, Friedreich's ataxia, and Duchenne muscular dystrophy. In some embodiments, depending on the condition to be treated, different oligonucleotides may be used in such complexes.

Further aspects of the disclosure, including a description of defined terms, are provided below.

### I. Definitions

Administering: As used herein, the terms “administering” or “administration” means to provide a complex to a subject in a manner that is physiologically and/or pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody, e.g., a full-length IgG. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (E), gamma (γ) or mu (p) heavy chain. In some embodiments, the heavy chain of an antibody described herein can comprise a human alpha (a), delta (A), epsilon (E), gamma (γ) or mu (p) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation. O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure covalently linked to a linker polypeptide or an immunoglobulin constant domain. Linker polypeptides comprise two or more amino acid residues joined by peptide bonds and are used to link one or more antigen binding portions. Example linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino add substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferring receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Disease allele: As used herein, the term “disease allele” refers to any one of alternative forms (e.g., mutant forms) of a gene for which the allele is correlated with and/or directly or indirectly contributes to, or causes, disease. A disease allele may comprise gene alterations including, but not limited to, insertions (e.g., disease-associated repeats described below), deletions, missense mutations, nonsense mutations and splice-site mutations relative to a wild-type (non-disease) allele. In some embodiments, a disease allele has a loss-of-function mutation. In some embodiments, a disease allele has a gain-of-function mutation. In some embodiments, a disease allele encodes an activating mutation (e.g., encodes a protein that is constitutively active). In some embodiments, a disease allele is a recessive allele having a recessive phenotype. In some embodiments, a disease allele is a dominant allele having a dominant phenotype.

Disease-associated-repeat: As used herein, the term “disease-associated-repeat” refers to a repeated nucleotide sequence at a genomic location for which the number of units of the repeated nucleotide sequence is correlated with and/or directly or indirectly contributes to, or causes, genetic disease. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, embodiments, the disease-associated-repeat comprises CAG repeats. CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or an extracellular domain, which may optionally further comprise a ligand-binding domain. In some embodiments, a cell surface receptor becomes internalized by a cell after ligand binding. In some embodiments, a ligand may be a muscle-targeting protein or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Mixed-mode resin: As used herein, the term “mixed-mode resin” refers to a chromatographic resin or material for use in purification, separation, and/or isolation of biomolecules that comprises positively-charged metal sites and negatively charged ionic sites. In some embodiments, the metal sites comprise calcium. In some embodiments, the negatively charged ionic sites comprise phosphate, sulfate, fluoride, or chloride. In some embodiments, the metal sites comprise calcium and the negatively charged ionic sites comprise phosphate, and optionally sulfate, fluoride, or chloride. In some embodiments, a mixed-mode resin is an apatite resin. In some embodiments, an apatite resin is hydroxyapatite resin, ceramic hydroxyapatite resin, hydroxyfluoroapatite resin, fluoroapatite resin, or chlorapatite resin. In some embodiments, apatite resin comprises minerals of the formula: Ca₁₀(PO₄)₆(OH)₂. In some embodiments, apatite resin comprises minerals of the formula: Ca₁₀(PO₄)₆F₂. In some embodiments, apatite resin comprises minerals of the formula: Ca₁₀(PO₄)₆Cl₂.

Molecular payload: As used herein, the term “molecular payload” refers to a molecule or species that functions to modulate a biological outcome. In some embodiments, a molecular payload is covalently linked to, or otherwise associated with a muscle-targeting agent. In some embodiments, the molecular payload is a small molecule, a protein, a peptide, a nucleic acid, or an oligonucleotide. In some embodiments, the molecular payload is a hydrophobic small molecule. In some embodiments, the molecular payload is an oligonucleotide (e.g., charge-neutral oligonucleotide (e.g., a phosphorodiamidate morpholino oligomer (PMO)) or charged oligonucleotide). In some embodiments, the molecular payload functions to modulate the transcription of a DNA sequence, to modulate the expression of a protein, or to modulate the activity of a protein. In some embodiments, the molecular payload is an oligonucleotide, e.g., an oligonucleotide that comprises a strand having a region of complementarity to a target gene.

Muscle Disease Gene: As used herein, the term “muscle disease gene” refers to a gene having a least one disease allele correlated with and/or directly or indirectly contributing to, or causing, a muscle disease. In some embodiments, the muscle disease is a rare disease, e.g., as defined by the Genetic and Rare Diseases Information Center (GARD), which is a program of the National Center for Advancing Translational Sciences (NCATS). In some embodiments, the muscle disease is a rare disease that is characterized as affecting fewer than 200,000 people. In some embodiments, the muscle disease is a single-gene disease. In some embodiments, a muscle disease gene is a gene listed in Table 1.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is covalently linked to a molecular payload. In some embodiments, the muscle-targeting agent is a muscle targeting protein (e.g., an antibody)

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capably of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of a oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a muscle disease (e.g., any of the diseases provided in Table 1).

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

An example human transferrin receptor amino acid sequence, corresponding to NCBI sequence NP_003225.2 (transferrin receptor protein 1 isoform 1, Homo sapiens) is as follows:

(SEQ ID NO: 1)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 2)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 3)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 4)
MMDQARSAFSNLFGGEPLSYTRFSLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLQWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF.

Unlinked molecular payload: As used herein, the term “unlinked molecular payload” refers to free molecular payload (e.g., oligonucleotide or small molecule) or excess molecular payload (e.g., oligonucleotide or small molecule) that is present in solution, e.g., following a conjugation reaction to generate complexes comprising protein linked to molecular payload (e.g., oligonucleotide or small molecule). In some embodiments, an unlinked molecular payload (e.g., oligonucleotide or small molecule) is not linked to a protein (e.g., antibody). In some embodiments, an unlinked molecular payload (e.g., oligonucleotide or small molecule) is not linked to any other moieties. In some embodiments, an unlinked molecular payload (e.g., oligonucleotide or small molecule) is linked to a functional group but is not linked to a protein (e.g., an antibody) to form a complex. In some embodiments, an unlinked molecular payload (e.g., oligonucleotide or small molecule) is linked to a linker or a portion of a linker but is not linked to a protein (e.g., an antibody) to form a complex.

Unlinked protein: As used herein, the term “unlinked protein” refers to free protein or excess protein (e.g., free antibody or excess antibody) that is present in solution, e.g., following a conjugation reaction to generate complexes comprising protein linked to molecular payload. In some embodiments, the unlinked protein (e.g., antibody) is not chemically modified. In some embodiments, the unlinked (e.g., antibody) is chemically modified. In some embodiments, the unlinked (e.g., antibody) is chemically modified to comprise a functional group but is not linked to a molecular payload (e.g., an oligonucleotide or a small molecule). In some embodiments, the functional group is for conjugation to the molecular payload (e.g., via click chemistry). In some embodiments, the functional group is an alkyne group. In some embodiments, the unlinked (e.g., antibody) comprises an alkyne group.

Complex: As used herein, the term “complex” refers to conjugates comprising a protein (e.g., antibody) covalently linked to one or more molecular payloads (e.g., a therapeutic agent such as a small molecule or an oligonucleotide). In some embodiments, the protein in the complex comprises a targeting agent (e.g., an antibody). In some embodiments, the targeting agent targets muscle (e.g., an anti-transferrin receptor antibody).

Process: As used herein, the term “process” includes but is not limited to the production (e.g., via conjugation), isolation (e.g., from the reaction mixture), and/or (e.g., and) modification of the complexes described herein.

Alkyne: As used herein, the term “alkyne” refers an unsaturated hydrocarbon containing at least one carbon-carbon triple bond.

Charged oligonucleotide: As used herein, the term “charged oligonucleotide” refers to an oligonucleotide analog comprising a backbone that has a net negative or net positive charge at a physiological pH (e.g., pH 7.35-pH 7.45). In some embodiments, a charged oligonucleotide has a net negative charge at a physiological pH (referred to herein as negatively charged oligonucleotide). In some embodiments, a charged oligonucleotide has a net positive charge at a physiological pH (referred to herein as positively charged oligonucleotide). In some embodiments, a charged oligonucleotide comprises a phosphodiester backbone that has a net negative charge at physiological pH. In some embodiments, a charged oligonucleotide comprises a phosphothioate backbone that has a net negative charge at physiological pH.

Charge-neutral oligonucleotide: As used herein, the term “charge-neutral oligonucleotide” refers to oligonucleotide analogs comprising charge-neutral backbones at a physiological pH (e.g., pH 7.35-pH 7.45). Examples of charge-neutral oligonucleotides include, without limitation, phosphorodiamidate morpholino oligomers (PMOs) and peptide nucleic acids (PNA), e.g., as described in Jarver et al., (Nucleic Acid Therapeutics, Vol. 25, No. 2, 2015), incorporated herein by reference.

Organic Solvent: As used herein, the term “organic solvent” refers to a carbon-based substance that is capable of dissolving other substances. By being carbon based, organic solvents have carbon atoms present in the structure of their compound. Non-limiting examples of organic solvents that can be used in accordance with the present disclosure include, without limitation, Dimethylacetamide (DMA), isopropyl alcohol (IPA), dimethyl sulfoxide (DMSO), acetonitrile (ACN), or propylene glycol (PG).

% (v/v): As used herein, the term “% (v/v)” refers to the % of the volume of the one component of a mixture (e.g., the organic solvent) in the entire volume of the mixture.

Drug-to-antibody Ratio (DAR): As used herein, the term “drug-to-antibody ratio (DAR)” refers to the number of drugs conjugated to the antibodies. This DAR number can vary with the nature of the antibody and of the drug used along with the experimental conditions used for the conjugation (ratio of antibody and molecular payload in the starting reaction material, the reaction time, the nature of the solvent and of the cosolvent if any). The DAR that is determined is a mean value. One example of a method that can be used to determine the DAR is described in Dimitrov et al., 2009, Therapeutic Antibodies and Protocols, vol 525, 445, Springer Science, incorporated herein by reference.

Intermediate: As used herein, the term “intermediate” refers to molecules that are generated doing the method of producing the complexes before the complex is obtained. In some embodiments, an intermediate comprises an oligonucleotide linked to a linker (e.g., cleavable linker, e.g., a val-cit linker (i.e., a cleavable linker comprising a valine-citrulline sequence). In some embodiments, an intermediate comprises an oligonucleotide covalently linked to a linker (e.g., cleavable linker, e.g., a val-cit linker) that is covalently linked to a compound comprising a bicyclononyne. In some embodiments, an intermediate comprises an antibody covalently linked a compound comprising a bicyclononyne.

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleotides and oligonucleotides comprising the 2′-modified nucleotides have increased affinity to a target sequences, relative to an unmodified oligonucleotide. Examples of structures of 2′-modified nucleosides are provided below:

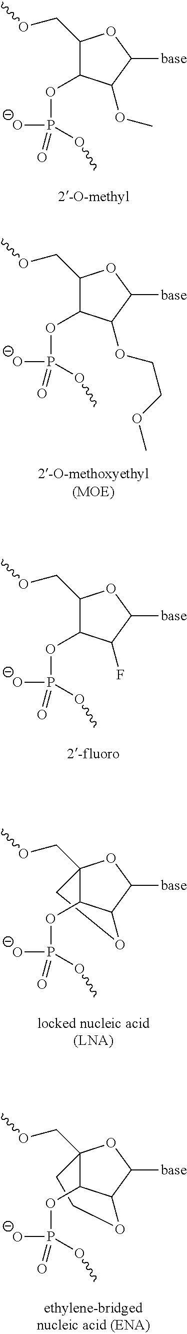

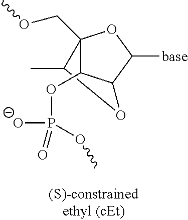

### II. Methods of Processing Complexes

In some aspects, the present disclosure provides methods of processing (e.g., producing, isolating) complexes that comprise a protein, e.g, an antibody, covalently linked to one or more molecular payloads (e.g., charge-neutral oligonucleotide or charged oligonucleotide).

In some aspects, the present disclosure provides methods of producing complexes that comprise a protein (e.g., an antibody) covalently linked to one or more oligonucleotides (e.g., charge-neutral oligonucleotide or charged oligonucleotide). In some embodiments, the methods comprise (i) covalently linking an oligonucleotide to a cleavable linker (e.g., val-cit linker) to obtain a first intermediate. In some embodiments, the methods further comprise (ii) covalently linking the first intermediate obtained in step (i) with a bicyclononyne compound to obtain a second intermediate. In some embodiments, the methods further comprise (iii) covalently linking the second intermediate obtained in step (ii) to an antibody to obtain the complexes. In some embodiments, the bicyclononyne compound is present in the reaction of step (iii) in an amount that is 10% or less or 5% or less (e.g., less than 5%, less than 4%, less than 3%, less than 2%, less than 1%, less than 0.5%, or less than 0.1%) of the starting amount of the bicyclononyne compound in step (ii). In some embodiments, the cleavable linker (e.g., val-cit linker) is linked to the 5′ end of the oligonucleotide. In some embodiments, the cleavable linker (e.g., val-cit linker) is linked to the oligonucleotide via additional chemical moieties. In some embodiments, step (iii) results in a lysine residue of the antibody being linked to the second intermediate.

In some embodiments, method of producing complexes comprises (i) covalently linking an oligonucleotide to a cleavable linker (e.g., val-cit linker) to obtain a first intermediate; (ii) covalently linking the first intermediate obtained in step (i) with a bicyclononyne compound to obtain a second intermediate; and (iii) covalently linking the second intermediate obtained in step (ii) to an antibody to obtain the complexes; wherein the bicyclononyne compound is present in the reaction of step (iii) in an amount that is 10% or less or 5% or less (e.g., less than 5%, less than 4%, less than 3%, less than 2%, less than 1%, less than 0.5%, or less than 0.1%) of the starting amount of the compound in step (ii). In this method, the cleavable linker (e.g., val-cit linker) is optionally linked to the 5′ end of the oligonucleotide. In some embodiments, the cleavable linker (e.g., val-cit linker) is linked to the oligonucleotide via additional chemical moieties. Further, in some embodiments, step (iii) results in a lysine residue of the antibody being linked to the second intermediate.

In some embodiments, the molecular payload is a hydrophobic small molecule. In some embodiments, the molecular payload is an oligonucleotide. In some embodiments, the molecular payload is a charge-neutral oligonucleotide. In some embodiments, the molecular payload is a charged oligonucleotide. In some embodiments, the oligonucleotide is a single stranded oligonucleotide (e.g., a charge-neutral single stranded oligonucleotide or a charged single stranded oligonucleotide). In some embodiments, the charge-neutral stranded oligonucleotide is an antisense oligonucleotide. In some embodiments, the charge-neutral oligo nucleotide is a phosphorodiamidate morpholino oligomer (PMO). In some embodiments, the charge-neutral oligo nucleotide is a peptide nucleic acid (PNA). In some embodiments, the charged oligonucleotide is a gapmer. In some embodiments, the antibody is covalently linked to the 5′ end of the single stranded oligonucleotide (e.g., gapmer or PMO). In some embodiments, the antibody is covalently linked to the 3′ end of the single stranded oligonucleotide (e.g., gapmer or PMO). In some embodiments, the antibody is covalently linked to the 5′ end of an antisense oligonucleotide (e.g., gapmer or PMO).

In some embodiments, the single stranded oligonucleotide is one strand of a double stranded oligonucleotide. In some embodiments, one strand of the double stranded oligonucleotide can be covalently conjugated to the antibody and the complexes can be isolated using the method described herein, followed by annealing of the other strand of the double stranded oligonucleotide. In some embodiments, the double stranded oligonucleotide is an siRNA and the sense strand is covalently linked to the antibody (e.g., at the 3′ end or at the 5′ end). In some embodiments, the complexes purified using the methods described herein comprise an antibody covalently linked to the 3′ end of the sense strand of a siRNA. The antisense strand of the siRNA can be annealed to the sense strand post purification.

In some embodiments, the oligonucleotide comprises at least one modified internucleotide linkage. In some embodiments, the at least one modified internucleotide linkage is a phosphorothioate linkage. In some embodiments, the oligonucleotide comprises one or more modified nucleotides. In some embodiments, the modified nucleotide comprises 2′-O-methoxyethylribose (MOE), locked nucleic acid (LNA), a 2′-fluoro modification, or a morpholino modification.

In some embodiments, the oligonucleotide is a single stranded oligonucleotide (e.g., an antisense oligonucleotide) comprising a modified nucleotide comprising 2′-O-methoxyethylribose (MOE), locked nucleic acid (LNA), a 2′-fluoro modification, or a morpholino modification. In some embodiments, the antisense oligonucleotide is a gapmer comprising a modified nucleotide comprising 2′-O-methoxyethylribose (MOE), locked nucleic acid (LNA), or a 2′-fluoro modification. In some embodiments, the antisense oligonucleotide is a phosphorodiamidate Morpholino oligomer (PMO). The antisense oligonucleotide may comprise more than one type of modifications described herein, e.g., having MOE and 2′-fluoro modifications.

In some embodiments, the oligonucleotide is a single stranded oligonucleotide (e.g., one strand of a double stranded RNA such as siRNA or an antisense oligonucleotide) comprising a modified nucleotide comprising 2′-O-methoxyethylribose (MOE), locked nucleic acid (LNA), or a 2′-fluoro modification. In some embodiments, the oligonucleotide is the sense strand of a siRNA comprising a modified nucleotide comprising 2′-O-methoxyethylribose (MOE), locked nucleic acid (LNA), or a 2′-fluoro modification. In some embodiments, the oligonucleotide is the antisense strand of a siRNA comprising a modified nucleotide comprising 2′-O-methoxyethylribose (MOE), locked nucleic acid (LNA), or a 2′-fluoro modification. The sense and antisense strand of the siRNA may comprise the same types or different types of modifications described herein. One or both strands of the siRNA may comprise more than one type of modifications described herein, e.g., having MOE and 2′-fluoro modifications.

In some embodiments, the method of producing complexes comprising an antibody covalently linked to one or more oligonucleotides (e.g., charge-neutral oligonucleotides or charged oligonucleotides) comprises:

(i) covalently linking the oligonucleotide (e.g., charge-neutral oligonucleotides or charged oligonucleotides) to a linker of Formula (A):

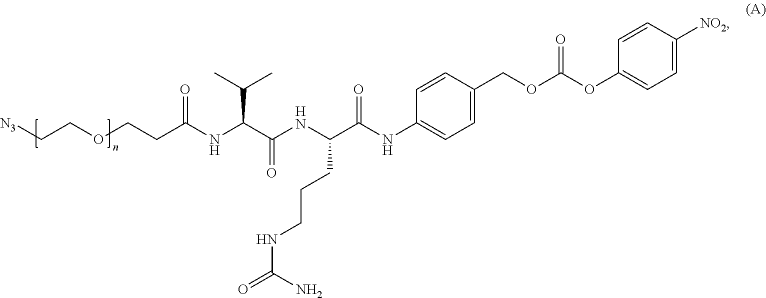

wherein n is 0-15 (e.g., 3): to provide a modified oligonucleotide of Formula (B):

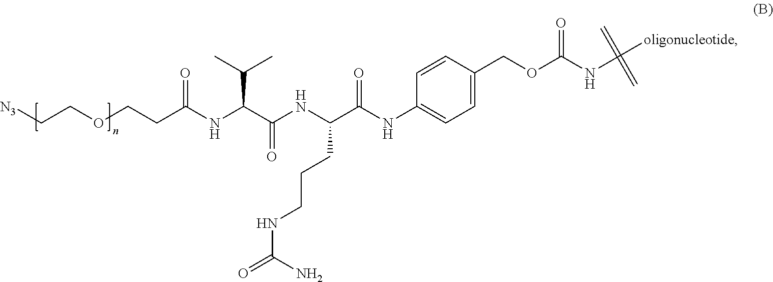

wherein n is 0-15 (e.g., 3);

(ii) contacting the modified oligonucleotide of Formula (B) with a compound of Formula (C):

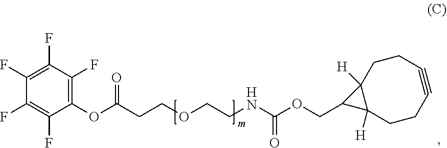

wherein m is 0-15 (e.g., 4); to provide a modified oligonucleotide of Formula (D):

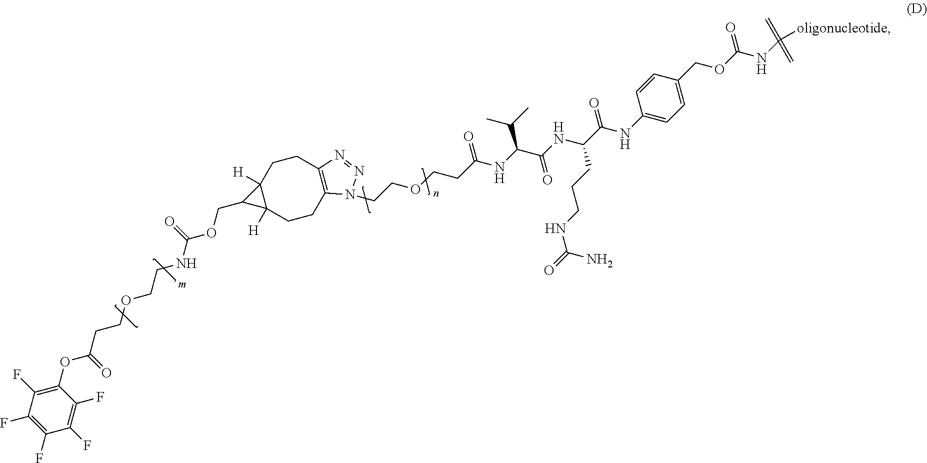

wherein n is 0-15 (e.g., 3) and m is 0-15 (e.g., 4); and

(iii) contacting the modified oligonucleotide of Formula (D) with an antibody to provide a complex of Formula (E):

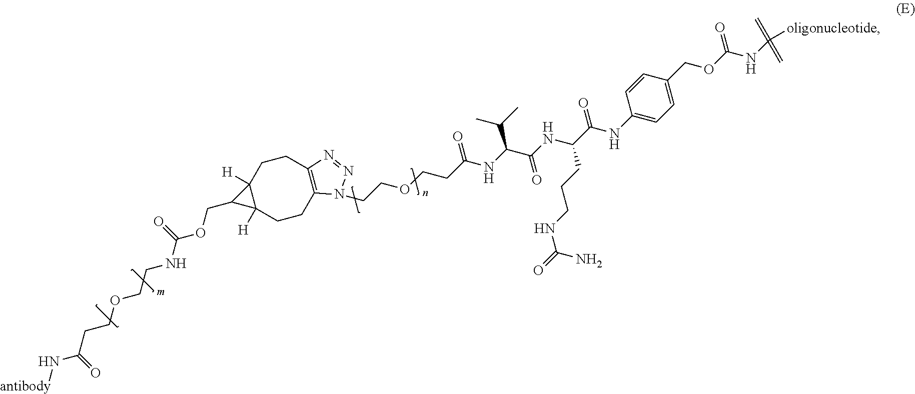

wherein n is 0-15 (e.g., 3) and m is 0-15 (e.g., 4).

In some embodiments, the methods of producing the complexes described herein produce a reaction mixture comprising the complexes, unlinked oligonucleotides (e.g., charge-neutral oligonucleotides or charged oligonucleotides), and/or unlinked antibodies. In some embodiments, the reaction mixture comprises complexes of Formula (E):

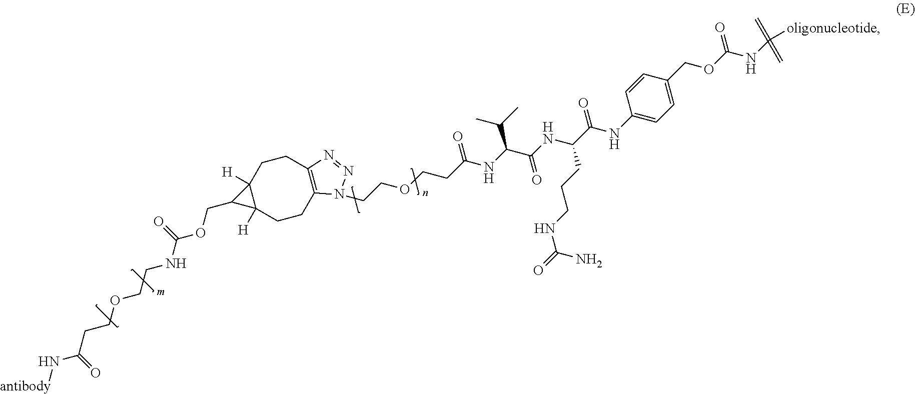

wherein n is 0-15 (e.g., 3) and m is 0-15 (e.g., 4); unlinked oligonucleotides of Formula (B):

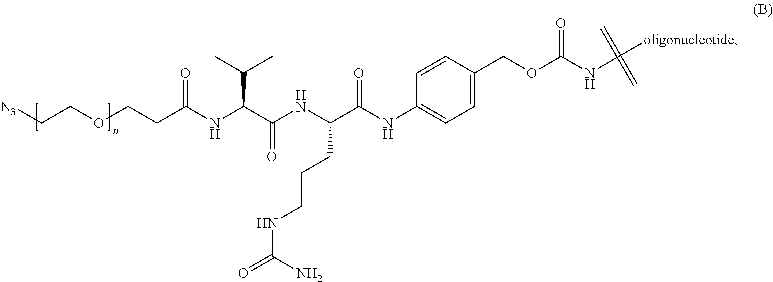

wherein n is 0-15 (e.g., 3); and unlinked antibodies. In some embodiments, the compound of Formula (C) is present in the reaction mixture of step (iii) in an amount that is 10% or less or 5% or less (e.g., less than 5%, less than 4%, less than 3%, less than 2%, less than 1%, less than 0.5%, or less than 0.1%) of the starting amount of the compound of Formula (C) in the reaction of step (ii), optionally wherein the compound of Formula (C) is contacted with the antibody in step (iii) to form, in the reaction mixture, unlinked antibodies of Formula (F):

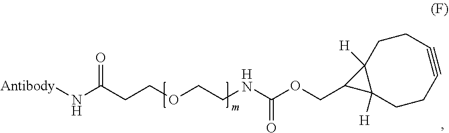

wherein m is 0-15 (e.g., 4).

In some embodiments, the methods of producing complexes each comprising an antibody covalently linked to one or more oligonucleotides (e.g., charge-neutral oligonucleotides or charged oligonucleotides) comprise covalently linking an oligonucleotide to a linker (e.g., a cleavable linker (e.g., val-cit linker)) to obtain a first intermediate. In some embodiments, the methods further comprise covalently linking an antibody with a bicyclononyne compound to obtain a second intermediate. In some embodiments, the methods further comprise covalently linking the first intermediate and the second intermediate to obtain the complexes. In this method, the linker (e.g., val-cit linker) is optionally linked to the 5′ end of the oligonucleotide. In some embodiments, the linker (e.g., val-cit linker) is covalently linked to the oligonucleotide via additional chemical moieties. Further, in some embodiments, step (iii) results in a lysine residue of the antibody being covalently linked to the second intermediate.

In some embodiments, the method of producing complexes each comprising an antibody covalently linked to one or more oligonucleotides (e.g., charge-neutral oligonucleotides or charged oligonucleotides) comprises: (i) covalently linking an oligonucleotide to a linker (e.g., cleavable linker (e.g., val-cit linker)) to obtain a first intermediate; (ii) covalently linking an antibody with a compound comprising a bicyclononyne to obtain a second intermediate; and (iii) covalently linking the first intermediate obtained in step (i) and the second intermediate obtained in step (ii) to obtain the complexes. In this method, the linker (e.g., val-cit linker) is optionally linked to the 5′ end of the oligonucleotide. In some embodiments, the linker (e.g., val-cit linker) is covalently linked to the oligonucleotide via additional chemical moieties. Further, in some embodiments, step (iii) results in a lysine residue of the antibody being covalently linked to the second intermediate.

In some embodiments, the method of producing complexes comprising an antibody covalently linked to one or more oligonucleotides (e.g., charge-neutral oligonucleotides or charged oligonucleotides) comprises:

(i) covalently linking the oligonucleotide to a linker of Formula (A):

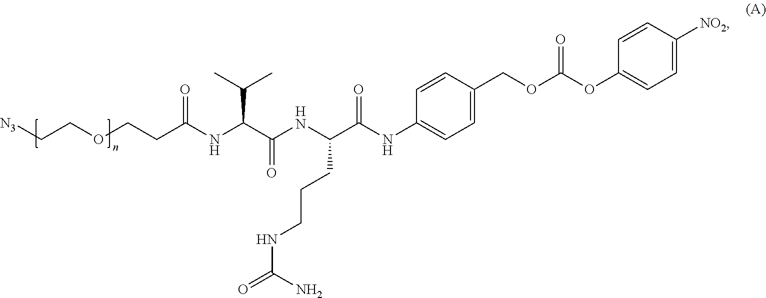

wherein n is 0-15 (e.g., 3): to provide a modified oligonucleotide of Formula (B):

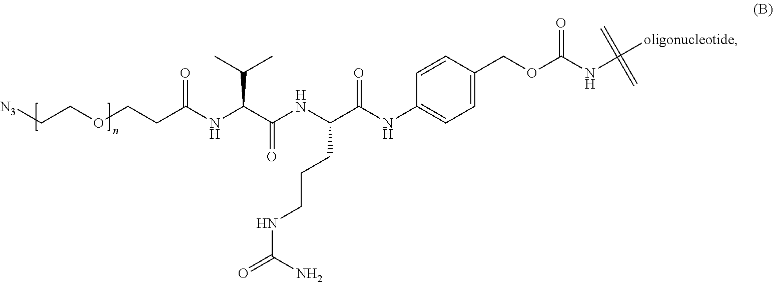

wherein n is 0-15 (e.g., 3);

(ii) covalently linking an antibody with a compound of Formula (C):

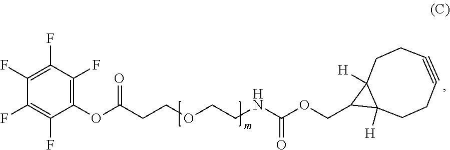

wherein m is 0-15 (e.g., 4); to provide a modified antibody of Formula (F):

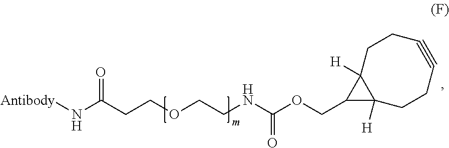

wherein n is 0-15 (e.g., 3) and m is 0-15 (e.g., 4); and

(iii) contacting the modified oligonucleotide of Formula (B) with the modified antibody of Formula (F) to provide a complex of Formula (E):

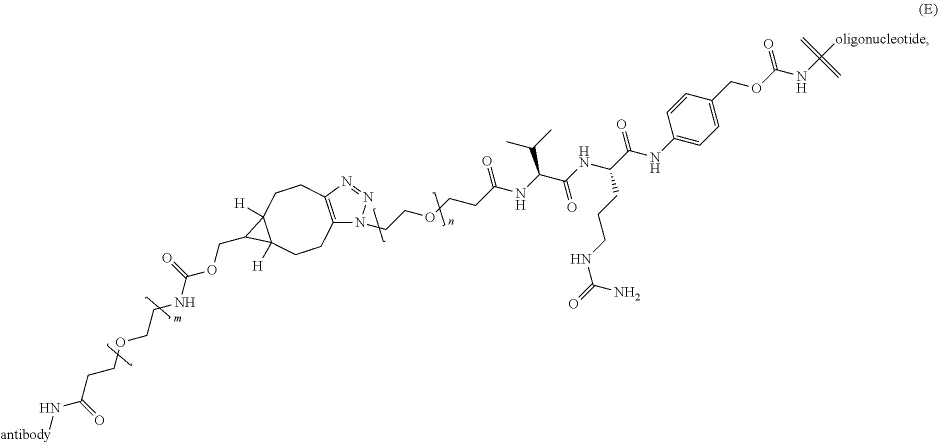

wherein n is 0-15 (e.g., 3) and m is 0-15 (e.g., 4).

In some embodiments, the methods of producing the complexes described herein produce a reaction mixture comprising the complexes, unlinked oligonucleotides, and/or unlinked antibodies. In some embodiments, the reaction mixture comprises complexes of Formula (E):

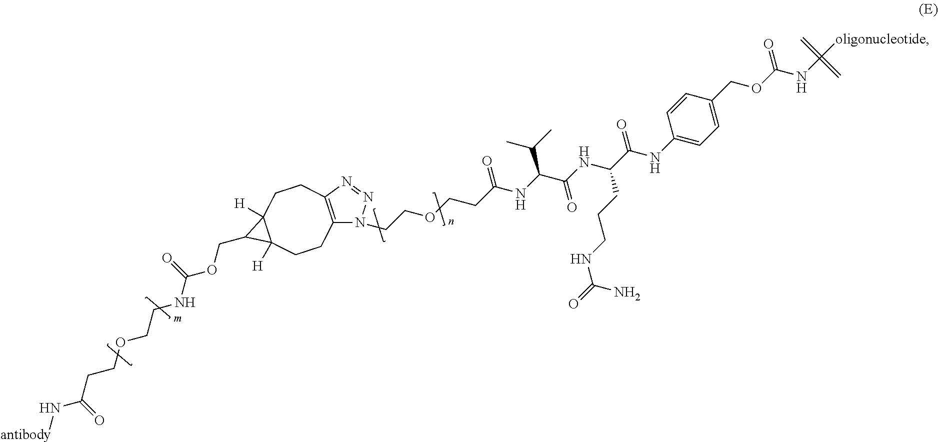

wherein n is 0-15 (e.g., 3) and m is 0-15 (e.g., 4); unlinked oligonucleotides of Formula (B):

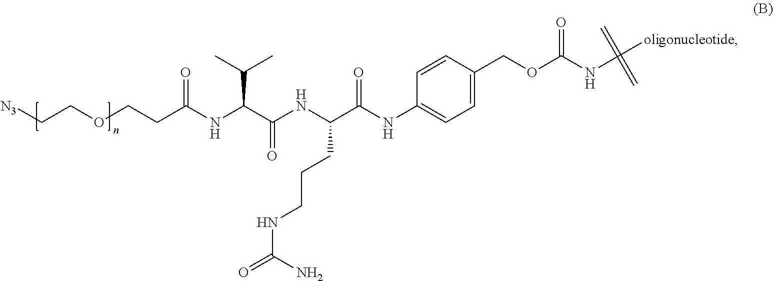

wherein n is 0-15 (e.g., 3); and unlinked antibodies of Formula (F)

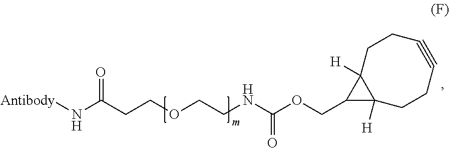

wherein m is 0-15 (e.g., 4).

In some embodiments, the oligonucleotide (e.g., charge-neutral oligonucleotide (e.g., PMO) or charged oligonucleotide) is 10-50 (e.g., 10-50, 10-40, 10-30, 10-20, 20-50, 20-40, 20-30, 30-50, 30-40, or 40-50) nucleotides in length. In some embodiments, the oligonucleotide (e.g., charge-neutral oligonucleotide (e.g., PMO) or charged oligonucleotide) is 15-30 (e.g., 15-30, 15-25, 15-20, 20-30, 20-25, or 25-30) nucleotides in length. In some embodiments, the oligonucleotide (e.g., charge-neutral oligonucleotide (e.g., PMO) or charged oligonucleotide) is 15, 16, 17, 18, 19, 20, 21, 22, 23, 24, 25, 26, 27, 28, 29, or 30 nucleotides in length. In some embodiments, the oligonucleotide (e.g., charge-neutral oligonucleotide (e.g., PMO) or charged oligonucleotide) is 30 nucleotides in length. In some embodiments, the oligonucleotide (e.g., charge-neutral oligonucleotide (e.g., PMO) or charged oligonucleotide) is covalently linked to the antibody via a lysine or a cysteine. In some embodiments, the oligonucleotide (e.g., charge-neutral oligonucleotide (e.g., PMO) or charged oligonucleotide) is covalently linked to the antibody via a linker (e.g., a linker that comprises a Val-cit linker). In some embodiments, the linker is of Formula (C):

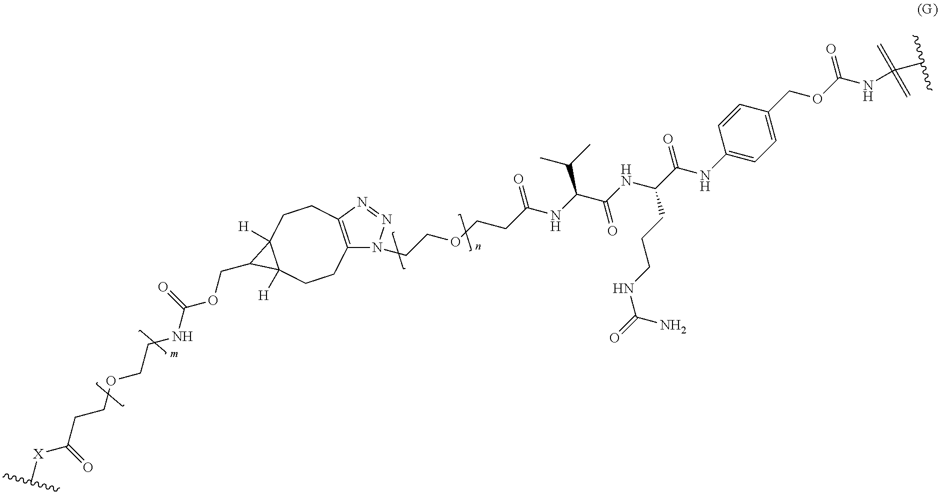

wherein n is 0-15 (e.g., 3) and m is 0-15 (e.g., 4). In some embodiments, n is 3 and/or (e.g., and) m is 4. In some embodiments, n is 3 and/or (e.g., and) m is 4. In some embodiments, X is NH (e.g., NH from an amine group of a lysine), S (e.g., S from a thiol group of a cysteine), or O (e.g., O from a hydroxyl group of a serine, threonine, or tyrosine) of the antibody.

In some embodiments, the complex is of Formula (E):

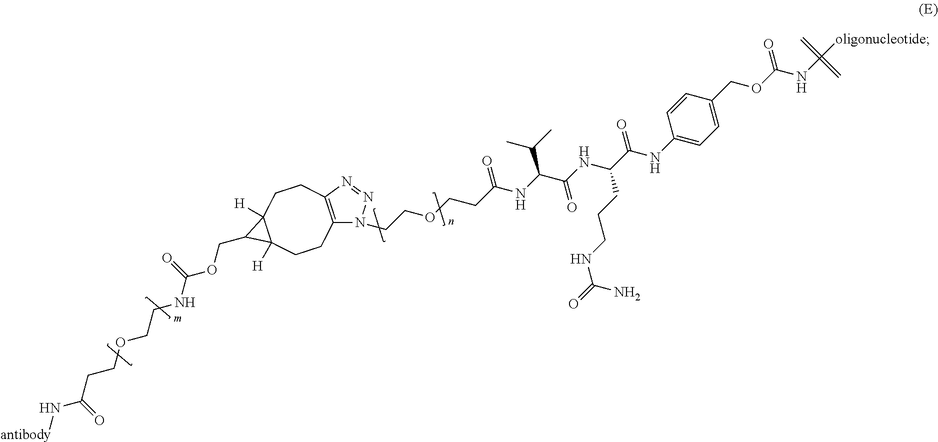

wherein n is 0-15 (e.g., 3) and m is 0-15 (e.g., 4). In some embodiments, the oligonucleotide is a charge-neutral oligonucleotide (e.g., PMO) or a charged oligonucleotide (e.g., gapmer).

In some aspects, the methods of isolating complexes described herein involve contacting a mixture comprising the complexes and unlinked molecular payloads (e.g., charge-neutral oligonucleotides or charged oligonucleotides) with a mixed-mode resin comprising positively-charged metal sites and negatively charged ionic sites, removing the unlinked molecular payloads (e.g., charge-neutral oligonucleotides or charged oligonucleotides), and eluting the adsorbed complexes from the mixed-mode resin. In some embodiments, the mixed-mode resin is an apatite resin. In some embodiments, an apatite resin is a hydroxyapatite resin, a ceramic hydroxyapatite resin, a hydroxyfluoroapatite resin, a fluoroapatite resin, or a chlorapatite resin.

In some embodiments, the mixture containing the complexes and the unlinked molecular payloads (e.g., charge-neutral oligonucleotides or charged oligonucleotides) that is subjected to the mixed-mode chromatography is a reaction mixture of a reaction that produces the complexes (e.g., via conjugation if the antibody and the molecular payloads). In some embodiments, the mixture the complexes and the unlinked molecular payloads (e.g., charge-neutral oligonucleotides or charged oligonucleotides) that is subjected to the mixed-mode chromatography is a reaction mixture of a reaction that produces the complexes (e.g., via conjugation if the antibody and the molecular payloads) that has not been subjected to any previous purification steps before the mixed-mode chromatography. In some embodiments, the complexes in the mixture that is subjected to the mixed-mode chromatography have an average drug to antibody ratio (DAR) of at least about 1.0 (e.g., about 1.0, 1.1, 1.2, 1.3, 1.4, 1.5, 1.6, 1.7, 1.8, 1.9, 2.0, 2.5, 3.0 or higher).

In some embodiments, complexes are substantially purified away from unlinked molecular payloads (e.g., charge-neutral oligonucleotides or charged oligonucleotides) using the mixed-mode resin chromatography described herein. In some embodiments, compositions of complexes following purification using the methods described herein do not comprise any detectable levels of unlinked oligonucleotide or unlinked protein.

In some embodiments, the methods described herein are suitable for isolating complexes comprising an antibody covalently linked to one or more oligonucleotides. In some embodiments, the antibody may be a full-length IgG, a Fab fragment, a Fab′ fragment, a F(ab′)2 fragment, a scFv, or a Fv fragment. The specific antibody sequences do not affect the purification outcome. In some embodiments, the antibody is an anti-transferrin receptor antibody (e.g., any of the anti-transferrin receptor antibodies listed in Table 2) or any antigen binding fragments thereof (e.g., a Fab fragment, a Fab′ fragment, a F(ab′)2 fragment, a scFv, or a Fv fragment).

A. Removal of Unlinked Charge-Neutral Oligonucleotide from Complex Using Mixed-Mode Resin

In some embodiments, it was shown herein that the use of mixed-mode resins that comprise positively-charged metal sites and negatively charged ionic sites (e.g., apatite resin, e.g., hydroxyapatite resin) are effective in removing unlinked molecular payload, particularly charge-neutral or hydrophobic molecular payloads (e.g., charge-neutral oligonucleotides or hydrophobic small molecules) from complexes, and that the use of organic solvent throughout the purification process significantly increases the yields of complexes free of unlinked molecular payloads. The mixed-mode resin purification method described herein is advantageous compared to other known methods of removing unlinked molecular payloads and/or excess salt (desalting). One such known method is size exclusion chromatography (SEC). The mixed-mode resin purification method is advantageous over SEC at least due to its scalability, and higher recovery rate. A recovery of at least 50% complexes were achieved using the mixed-mode resin method described herein, while SEC can only achieve 20-30% recovery of the complexes.

In some aspects, the present disclosure provides methods of processing (e.g., isolating) complexes each comprising an antibody covalently linked to one or more molecular payloads. In some embodiments, the molecular payload is a small molecule (e.g., a hydrophobic small molecule). In some embodiments, the molecular payload is an oligonucleotide (e.g., a charge-neutral oligonucleotide).

In some embodiments, the method of processing complexes described herein comprises: (i) contacting a mixture comprising an organic solvent, the complexes and unlinked molecular payload (e.g., charge-neutral oligonucleotides or hydrophobic small molecules) with a mixed-mode resin that comprises positively-charged metal sites and negatively charged ionic sites, under conditions in which the complexes adsorb to the mixed-mode resin, and (ii) eluting the complexes from the mixed-mode resin under conditions in which the complexes dissociate from the mixed-mode resin. In some embodiments, the mixture of step (i) further comprises no more than 30% (e.g., no more than 30%, no more than 25%, no more than 20%, no more than 15%, no more than 10%, no more than 9%, no more than 8%, no more than 7%, no more than 6%, no more than 5%, no more than 4%, no more than 3%, no more than 2%, no more than 1%, or less than 0.5%) of unlinked antibodies. In some embodiments, the mixture of step (i) further comprises undetectable levels of unlinked antibodies. In some embodiments, the mixture of step (i) is a reaction mixture that produces the complexes. In some embodiments, the mixture of step (i) is a reaction mixture that produces the complexes that has not been subjected to any previous steps of purification. In some embodiments, the mixture of step (i) comprises trace amounts of unlinked antibodies that comprise an alkyne group.

In some embodiments, the molecular payload (linked or unlinked) interacts with the mixed-mode resin non-specifically, affecting the yield of the complex. It was demonstrated herein that, including an organic solvent in the mobile phase of the mixed-mode chromatography effectively reduced/eliminated the non-specific interaction between charge-neutral oligonucleotides and the mixed-mode resin. In some embodiments, reducing the non-specific interaction between molecular payloads (e.g., charge-neutral oligonucleotides or hydrophobic small molecules) results in increased (e.g., by at least 20%, at least 30%, at least 40%, at least 50%, at least 60%, at least 70%, at least 80%, at least 90%, at least 100%, at least 2-fold, at least 5-fold, or more) yields of complexes, compared to without reducing such non-specific interaction.

Organic solvents commonly used in chromatography methods can be used throughout the methods described herein. In some embodiments, the organic solvent used in step (i) of the methods of processing complexes described herein is Dimethylacetamide (DMA), isopropyl alcohol (IPA), dimethyl sulfoxide (DMSO), acetonitrile (ACN), or propylene glycol (PG). In some embodiments, the organic solvent used in step (i) of the methods described herein is Dimethylacetamide (DMA). In some embodiments, the organic solvent used in step (i) of the methods described herein is isopropyl alcohol (IPA). In some embodiments, the organic solvent used in step (i) of the methods described herein is dimethyl sulfoxide (DMSO). In some embodiments, the organic solvent used in step (i) of the methods described herein is acetonitrile (ACN). In some embodiments, the organic solvent used in step (i) of the methods described herein is propylene glycol (PG).

In some embodiments, the organic solvent is at 2-50% (v/v) in the mixture in step (i). For example, the organic solvent may be at 2-50% (v/v), 5-50% (v/v), 10-50% (v/v), 20-50% (v/v), 30-50% (v/v), 40-50% (v/v), 2-40% (v/v), 5-40% (v/v), 10-40% (v/v), 20-40% (v/v), 30-40% (v/v), 2-30% (v/v), 5-30% (v/v), 10-30% (v/v), 20-30% (v/v), 2-20% (v/v), 5-20% (v/v), 10-20% (v/v), 2-10% (v/v). 5-10% (v/v), 5-20% (v/v), 5-15% (v/v), 5-10% (v/v), 10-15% (v/v), or 15-20% (v/v) in the mixture in step (i) of the methods described herein. In some embodiments, the organic solvent is at 5-20% (v/v) in the mixture in step (i). In some embodiments, the organic solvent is at 5% (v/v), 6% (v/v), 7% (v/v), 8% (v/v), 9% (v/v), 10% (v/v), 11% (v/v), 12% (v/v), 13% (v/v), 14% (v/v), 15% (v/v), 16% (v/v), 17% (v/v), 18% (v/v), 19% (v/v), or 20% (v/v) in the mixture in step (i) of the methods described herein. In some embodiments, organic solvents at more than 20% (v/v) may be used in the mixture of step (i) in the methods described herein. In some embodiments, the organic solvent is at 15% (v/v) in the mixture in step (i) of the methods described herein. In some embodiments, the organic solvent is at 30% (v/v) in the mixture in step (i) of the methods described herein.

In some embodiments, the conditions in step (i) under which the complexes adsorb are achieved by including phosphate ions and/or chloride ions in the mixture in step (i) at a concentration that allows the complexes to adsorb to the mixed-mode resin. In some embodiments, the mixture in step (i) has a pH of about 5.0-8.0 (e.g., about 5.0, 5.5, 6.0, 6.5, 7.0, 7.5, 8.0). In some embodiments, the mixture in step (i) has a pH of about 5.0-6.0 (e.g., about 5.0, 5.1, 5.2, 5.3, 5.4, 5.5, 5.6, 5.7, 5.8, 5.9, or 6.0). In some embodiments, the mixture in step (i) of the methods described herein does not comprise a phosphate ion or a chloride ion. In some embodiments, the mixture in step (i) of the methods described herein further comprises up to 10 mM phosphate ion. In some embodiments, the mixture in step (i) of the methods described herein further comprises up to 10 mM (e.g., up to 10 mM, or up to 5 mM) phosphate ion, and optionally in some embodiments, the mixture in step (i) of the methods described herein further comprises up to 20 mM (e.g., up to 20 mM, up to 15 mM, up to 10 mM, or up to 5 mM) chloride ion. In some embodiments, the mixture in step (i) of the methods described herein further comprises 5-10 mM (e.g., 5, 6, 7, 8, 9, or 10 mM) phosphate ion, and optionally in some embodiments, the mixture in step (i) of the methods described herein further comprises 5-20 mM chloride ions (e.g., 5, 6, 7, 8, 9, 10, 11, 12, 13, 14, 15, 16, 17, 18, 19, or 20 mM). Under these conditions, the unlinked molecular payloads (e.g., charge-neutral oligonucleotides or hydrophobic small molecules) remain in the flow through and do not adsorb to the mixed-mode resin.

In some embodiments, the unlinked molecular payloads (e.g., charge-neutral oligonucleotides or hydrophobic small molecules) do not adsorb to the mixed-mode resin in step (i). In some embodiments, less than 5% (e.g., less than 5%, less than 4%, less than 3%, less than 2%, less than 1%, or less than 0.5%) of unlinked molecular payloads (e.g., charge-neutral oligonucleotides or hydrophobic small molecules) adsorb to the mixed-mode resin in step (i). In some embodiments, less than 5% (e.g., less than 5%, less than 4%, less than 3%, less than 2%, less than 1%, or less than 0.5%) of unlinked molecular payloads (e.g., charge-neutral oligonucleotides or hydrophobic small molecules) interact non-specifically with the mixed-mode resin.

In some embodiments, the mixed-mode resin may further be washed between step (i) and step (ii) under conditions that remove unlinked molecular payloads (e.g., charge-neutral oligonucleotides or hydrophobic small molecules) that are loosely bound but not adsorbed to the mixed-mode resin. In some embodiments, the washing step is performed using a washing solution. In some embodiments, the washing solution comprises an organic solvent. In some embodiments, the organic solvent used in the washing solution is Dimethylacetamide (DMA), isopropyl alcohol (IPA), dimethyl sulfoxide (DMSO), acetonitrile (ACN), or propylene glycol (PG). In some embodiments, the organic solvent used in the washing solution is Dimethylacetamide (DMA). In some embodiments, the organic solvent used in the washing solution is isopropyl alcohol (IPA). In some embodiments, the organic solvent used in the washing solution is dimethyl sulfoxide (DMSO). In some embodiments, the organic solvent used in the washing solution is acetonitrile (ACN). In some embodiments, the organic solvent used in the washing solution is propylene glycol (PG).

In some embodiments, the organic solvent is at 5%-20% (v/v) in the washing solution. For example, the organic solvent may be at 5%-20% (v/v), 5%-15% (v/v), 5%-10% (v/v), 10%-20% (v/v), 10%-15% (v/v), or 15%-20% (v/v) in the washing solution. In some embodiments, the organic solvent is at 5% (v/v), 6% (v/v), 7% (v/v), 8% (v/v), 9% (v/v), 10% (v/v), 11% (v/v), 12% (v/v), 13% (v/v), 14% (v/v), 15% (v/v), 16% (v/v), 17% (v/v), 18% (v/v), 19% (v/v), or 20% (v/v) in the washing solution. In some embodiments, organic solvents at more than 20% (v/v) may be used in the washing solution. In some embodiments, the organic solvent is at 15% (v/v) in the washing solution. In some embodiments, the organic solvent is at 30% (v/v) in the washing solution.

In some embodiments, the washing solution comprises phosphate ions and/or chloride at a concentration that removes the loosely bound molecular payloads (e.g., charge-neutral oligonucleotides or hydrophobic small molecule) but does not dissociate the complexes from the mixed-mode resin. In some embodiments, the washing solution has a pH of 5.0-8.0 (e.g., about 5.0, 5.5, 6.0, 6.5, 7.0, 7.5, or 8.0). In some embodiments, the washing solution has a pH of 5.0-6.0 (e.g., 5.0, 5.1, 5.2, 5.3, 5.4, 5.5, 5.6, 5.7, 5.8, 5.9, or 6.0). In some embodiments, the washing solution does not comprise a phosphate ion or a chloride ion. In some embodiments, the washing solution further comprises up to 10 mM phosphate ion. In some embodiments, the washing solution further comprises up to 10 mM (e.g., up to 10 mM, or up to 5 mM) phosphate ion, and optionally in some embodiments, the washing solution further comprises up to 20 mM (e.g., up to 20 mM, up to 15 mM, up to 10 mM, or up to 5 mM) chloride ion. In some embodiments, the washing solution further comprises 5-10 mM (e.g., 5, 6, 7, 8, 9, or 10 mM) phosphate ion and optionally in some embodiments the washing solution further comprises 5-20 mM chloride ions (e.g., 5, 6, 7, 8, 9, 10, 11, 12, 13, 14, 15, 16, 17, 18, 19, or 20 mM).

In some embodiments, to elute the complexes from the mixed-mode resin, in step (ii), the mixed-mode and the bound complexes are subject to conditions that allow the dissociation of the complexes from the mixed-mode resin. In some embodiments, conditions in step (ii) that allow dissociation of the complexes are achieved by applying to the mixed-mode resin an elution solution comprising a higher concentration of phosphate ions and/or chloride ions, compared to the concentration of phosphate ions and/or chloride ions in the mixture of step (i) or in the washing solution. In some embodiments, the elution solution comprises a higher concentration of phosphate ions, compared to the concentration of phosphate in the mixture of step (i) or in the washing solution, and does not comprise chloride ions. The elution step may be done using an elution solution comprising a single phosphate ion concentration, or using an elution solution have a gradient of increasing phosphate ion concentration.

In some embodiments, the elution solution has a pH of about 6.5-8.5 (e.g., about 6.5, 7.0, 7.5, 8.0, or 8.5). In some embodiments, the elution solution has a pH of about 7.6-8.5 (e.g., about 7.6, 7.7, 7.8, 7.9, 8.0, 8.1, 8.2, 8.3, 8.4, or 8.5).

In some embodiments, the elution solution comprises an organic solvent. In some embodiments, the organic solvent used in the elution solution is Dimethylacetamide (DMA), isopropyl alcohol (IPA), dimethyl sulfoxide (DMSO), acetonitrile (ACN), or propylene glycol (PG). In some embodiments, the organic solvent used in the elution solution is Dimethylacetamide (DMA). In some embodiments, the organic solvent used in the elution solution is isopropyl alcohol (IPA). In some embodiments, the organic solvent used in the elution solution is dimethyl sulfoxide (DMSO). In some embodiments, the organic solvent used in the elution solution is acetonitrile (ACN). In some embodiments, the organic solvent used in the elution solution is propylene glycol (PG).

In some embodiments, the organic solvent is at 2-50% (v/v) in the elution solution. For example, the organic solvent may be at 2-50% (v/v), 5-50% (v/v), 10-50% (v/v), 20-50% (v/v), 30-50% (v/v), 40-50% (v/v), 2-40% (v/v), 5-40% (v/v), 10-40% (v/v), 20-40% (v/v), 30-40% (v/v), 2-30% (v/v), 5-30% (v/v), 10-30% (v/v), 20-30% (v/v), 2-20% (v/v), 5-20% (v/v), 10-20% (v/v), 2-10% (v/v), 5-10% (v/v), 5-20% (v/v), 5-15% (v/v), 5-10% (v/v), 10-15% (v/v), or 15-20% (v/v), in the elution solution. In some embodiments, the organic solvent is at 5-20% (v/v) in the elution solution. In some embodiments, the organic solvent is at 5% (v/v), 6% (v/v), 7% (v/v), 8% (v/v), 9% (v/v), 10% (v/v), 11% (v/v), 12% (v/v), 13% (v/v), 14% (v/v), 15% (v/v), 16% (v/v), 17% (v/v), 18% (v/v), 19% (v/v), or 20% (v/v) in the elution solution. In some embodiments, organic solvents at more than 20% (v/v) may be used in the elution solution. In some embodiments, the organic solvent is at 15% (v/v) in the elution solution. In some embodiments, the organic solvent is at 30% (v/v) in the elution solution.

In some embodiments, the elution solution of step (ii) comprises at least 30 mM phosphate ions. In some embodiments, the elution solution of step (ii) comprises at least 30 mM (e.g., at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM) phosphate ions. In some embodiments, the elution solution of step (ii) comprises at least 100 mM phosphate ions. In some embodiments, the elution solution of step (ii) comprises 100 mM phosphate ions.

In some embodiments, the elution solution comprises a gradually increasing concentration of phosphate ions. In some embodiments, the concentration of the phosphate ions increases from at least 10 mM (e.g., 10 mM, 15 mM, or 20 mM) to at least 100 mM (e.g., 100 mM, 150 mM, 200 mM, 250 mM, 300 mM, or higher) during step (ii). In some embodiments, the concentration of the phosphate ions increases from 10 mM to 100 mM during step (ii).

In some embodiments, applying the elution solution to the mixed mode resin dissociates at least 50% (e.g., at least 50%, at least 60%, at least 70%, at least 80%, at least 90%, at least 95%, or at least 99%) of the complexes from the mixed-mode resin. In some embodiments, applying the elution solution to the mixed mode resin dissociates 50%, 60%, 70%, 80%, 90%, 95%, 99%, or more of the of the complexes from the mixed-mode resin.

In some embodiments, the elution solution does not comprise chloride ions. In some embodiments, the elution solution may further comprise at least 50 mM (e.g., at least 50 mM, at least 60 mM, at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, or at least 200 mM) chloride ions.

In some embodiments, the method described herein further comprises collecting the dissociated complexes. In some embodiments, the method described herein further comprises preparing the complexes in a formulation buffer (e.g., by buffer exchange). In some embodiments, the buffer exchange can be performed via ultrafiltration/diafiltration (UF/DF), or tangential flow filtration (TFF).

In some aspects, the present disclosure provides methods of processing complexes each comprising an antibody covalently linked to one or more charge-neutral oligonucleotides, the method comprises:

(i) contacting a mixture comprising DMA or IPA at 15% (v/v), the complexes and unlinked charge-neutral oligonucleotides with hydroxyapatite (HA) resin that comprises positively-charged metal sites and negatively charged ionic sites, wherein the mixture has a pH of about 5.7 and optionally further comprises 10 mM of phosphate ions and/or (e.g., and) 20 mM of chloride ions;

(ii) washing the HA resin with a washing solution that has a pH of about 5.7 and comprises DMA or IPA at 15% (v/v), and optionally further comprises 10 mM of phosphate ions and/or (e.g., and) 20 mM of chloride ions;

(iii) eluting the complexes from the mixed-mode resin by applying an elution solution to the HA resin, wherein the elution solution has a pH of about 7.6 and comprises:
(a) DMA or IPA at 15% (v/v), and
(b) phosphate ions at 100 mM or a gradient of phosphate ions having a concentration range of 30 mM to 100 mM; and

(iv) collecting the elected complexes.

In some embodiments, purification of the anti-TfR Fab-oligonucleotide conjugate requires a two-part purification process. In some embodiments, the reaction mixture containing the complexes and unlinked Fabs and/or oligonucleotides is diluted (e.g., 1:3) in nuclease free water and the pH is adjusted (e.g., to 5.7) with the addition of appropriate buffer (e.g., MES buffer) at an appropriate concentration (e.g., about 50 mM). In some embodiments, a ceramic hydroxyapatite (HA) column is equilibrated using 15:85 v/v % of organic solvent DMA to 10 mM sodium phosphate at pH 5.8. In some embodiments, the crude reaction mixture is loaded onto the HA column to remove unconjugated oligonucleotide. In some embodiments, the HA column is washed with 15:85 v/v % of DMA in 10 mM sodium phosphate buffer (pH 5.8). In some embodiments, elution is initiated via a step gradient with 100 mM sodium phosphate, pH 7.6 buffer containing DMA at 15:85 v/v % at a flow rate of 5 mL/min. In some embodiments, the HA eluate is buffer exchanged into the final formulation. In some embodiments, the final purified anti-TfR Fab-oligonucleotide conjugate is analyzed (e.g. by SEC, SDS-PAGE densitometry, and/or BCA).

In some embodiments, the mixtures and solutions used in the method described herein further comprises counter ions for the phosphate ion and/or the chloride ions. In some embodiments, the counter ion for phosphate is a calcium, sodium, magnesium, potassium, or manganese. In some embodiments, the counter ion used in the methods described herein is sodium. In some embodiments, a source of phosphate ions is NaH₂PO₄, Na₂HPO₄, or Na₃PO₄. In some embodiments, the counter ion for chloride is a calcium, sodium, magnesium, potassium, or manganese. In some embodiments, a source of chloride ions is NaCl. One of skill in the art would readily understand that many other equivalent salts and ions may be used for the methods described herein.

In some embodiments, a wash solution and/or an eluent solution may further comprise a buffering agent in order to maintain a consistent pH. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

Any mixed-mode resin that comprises positively-charged metal sites and negatively charged ionic sites may be used in accordance with the present disclosure. In some embodiments, the mixed-mode resin used in the methods described herein is an apatite resin. In some embodiments, the apatite resin is a hydroxyapatite resin, a ceramic hydroxyapatite resin, a hydroxyfluoroapatite resin, a fluoroapatite resin, or a chlorapatite resin. An apatite resin may comprise apatite in any form and is typically used as a chromatographic solid phase in the separation and purification of biomolecules, e.g., complexes described herein, using affinity, ion exchange, hydrophobic interactions, or combinations thereof.

In some embodiments, a hydroxyapatite resin is a Bio-Gel HT resin, e.g., from Bio-Rad Laboratories, Inc. (Hercules, Calif., USA). In some embodiments, a ceramic hydroxyapatite resin is a Bio-Scale Mini CHT resin, e.g., from Bio-Rad Laboratories, Inc. In some embodiments, an apatite resin, e.g., ceramic hydroxyapatite, comprises spherical particles of apatite. In some embodiments, the spherical particles of apatite are about 10 microns to about 100 microns, about 25 microns to about 50 microns, about 20 microns, about 30 microns, about 40 microns, about 50 microns, about 60 microns, or about 80 microns in diameter. In some embodiments, an apatite resin, e.g., ceramic hydroxyapatite, is Type I (medium porosity and a high binding capacity) or Type 11 (larger porosity and a lower binding capacity). In some embodiments, an apatite particle may be used in admixture with another separation medium or support.

In some embodiments, a mixed-mode resin may be equilibrated prior to being contacted with a mixture of complex and unlinked molecular payloads (e.g., charge-neutral oligonucleotides or hydrophobic small molecules). In some embodiments, a mixed-mode resin is equilibrated using a wash solution, as described above. In some embodiments, a mixed-mode resin is equilibrated to bring the pH to about 5.0-8.0.

In some embodiments, a mixed-mode resin is packed into a column, e.g., a vertical column. In some embodiments, a column may be used under pressure, optionally pressure from top to bottom or bottom to top. In some embodiments, a column may be used without external pressure, e.g., using gravity flow only. In some embodiments, a mixed-mode resin is used as free resin, e.g., using a batch method. In some embodiments, a batch method may further comprise centrifugation and/or filtration steps following contacting of the resin with the mixture of complex and unlinked molecular payloads (e.g., charge-neutral oligonucleotides or hydrophobic small molecules).

In some embodiments, the complexes in the mixture of step (i) of the methods described herein and/or (e.g., and) eluted in step (ii) of methods described herein comprises an antibody covalently linked to 1-10 (e.g., 1, 2, 3, 4, 5, 6, 7, 8, 9, or 10) molecular payloads (e.g., charge-neutral oligonucleotides or hydrophobic small molecules). In some embodiments, at least 50% (e.g., at least 50%, at least 60%, at least 70%, at least 80%, at least 90% or more) of the complexes in the mixture of step (i) and/or (e.g., and) eluted in step (ii) of the methods described herein comprise an antibody covalently linked to 1-3 (e.g., 1, 2, or 3) molecular payloads (e.g., charge-neutral oligonucleotides or hydrophobic small molecules). In some embodiments, the complexes in the mixture of step (i) and/or (e.g., and) eluted in step (ii) of the methods described herein have an average drug to antibody ration (DAR) of at least about 1.5 (e.g., at least about 1.5, at least about 1.6, at least about 1.7, at least about 1.8, at least about 1.9, or at least about 2). In some embodiments, the eluent obtained from step (ii) comprises undetectable levels of unlinked molecular payloads (e.g., charge-neutral oligonucleotides or hydrophobic small molecules). In some embodiments, the eluent obtained from step (ii) comprises undetectable levels of unlinked antibodies.

B. Removal of Unlinked Charged Oligonucleotide from Complex Using Mixed-Mode Resin

In some embodiments, it was shown herein that the use of mixed-mode resins that comprise positively-charged metal sites and negatively charged ionic sites (e.g., apatite resin, e.g., hydroxyapatite resin) are effective in purifying complex away from unlinked oligonucleotide. This was a surprising finding in large part because no other purification strategy alternatives were able to remove essentially all unlinked oligonucleotide from compositions comprising protein-oligonucleotide complexes. Further, the mixed-mode resin purification method described herein is advantageous compared to other known methods of removing unlinked oligonucleotides and/or excess salt (desalting). One such known method is size exclusion chromatography (SEC). The mixed-mode resin purification method is advantageous over SEC at least due to its scalability, and higher recovery rate. A recovery of at least 90% complexes were achieved using the mixed-mode resin method described herein, while SEC can only achieve 20-30% recovery of the complexes.

In some embodiments, the method of isolating a complex or plurality of complexes each comprising an antibody covalently linked to one or more oligonucleotides (e.g., a charged oligonucleotide) described herein comprises: (i) contacting a mixture comprising the complexes and unlinked oligonucleotides with a mixed-mode resin that comprises positively-charged metal sites and negatively charged ionic sites, under conditions in which the complexes adsorb to the mixed-mode resin, and (ii) eluting the complexes from the mixed-mode resin under conditions in which the complexes dissociate from the mixed-mode resin. In some embodiments, the mixture in step (i) comprises trace amounts of unlinked antibodies that comprise an alkyne group. In some embodiments, the mixture in step (i) has not been subjected to purification prior to being contacted with the mixed-mode resin. As described herein, in some embodiments, the conditions in step (i) under which the complexes adsorb to the mixed-mode resin may be adjusted to allow or exclude the unlinked oligonucleotides from adsorbing to the mixed-mode resin.

In some embodiments, the conditions in step (i) under which the complexes adsorb to the mixed-mode resin does not allow the unlinked oligonucleotides from adsorbing to the mixed-mode resin, thus separating the complexes from the unlinked oligonucleotides, in some embodiments, the conditions are achieved by including phosphate ions and/or chloride ions in the mixture in step (i) at a concentration that allows the complexes but not the unlinked oligonucleotides to adsorb to the mixed-mode resin. In some embodiments, the mixture comprising the complexes and unlinked oligonucleotides further comprises up to 20 mM phosphate ion and/or up to 30 mM chloride ions. In some embodiments, the mixture comprising the complexes and unlinked oligonucleotides further comprises up to 20 mM (e.g., up to 20 mM, up to 15 mM, up to 10 mM, or up to 5 mM) phosphate ion. Additionally, in some embodiments, the mixture comprising the complexes and unlinked oligonucleotides further comprises up to 30 mM (e.g., up to 30 mM, up to 25 mM, up to 20 mM, up to 15 mM, up to 10 mM, or up to 5 mM) chloride ion. In some embodiments, the mixture comprising the complexes and unlinked oligonucleotides further comprises 5-20 mM (e.g, 5-20 mM, 5-15 mM, 5-10 mM, 10-20 mM, 10-15 mM, or 15-20 mM) phosphate ion and/or 5-30 mM chloride ions (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM). In some embodiments, the mixture comprising the complexes and unlinked oligonucleotides further comprises 20 mM, 15 mM, 10 mM, 5 mM, or 1 mM phosphate ion and/or 30 mM, 25 mM, 20 mM, 15 mM, 10 mM, or 5 mM chloride ion. In some embodiments, the mixture comprising the complexes and unlinked oligonucleotides further comprises 20 mM phosphate ion and/or 30 mM chloride ion, e.g. 20 mM phosphate ion and 30 mM chloride ion. In some embodiments, the mixture comprising the complexes and unlinked oligonucleotides further comprises up to 10 mM phosphate ions and/or up to 25 mM chloride ions. In some embodiments, the mixture comprising the complexes and unlinked oligonucleotides further comprises 5-10 mM phosphate ions and/or 5-25 mM chloride ions. In some embodiments, the mixture comprising the complexes and unlinked oligonucleotides further comprises 10 mM phosphate ions and/or 25 mM chloride ions, e.g., 10 mM phosphate ions and 25 mM chloride ions. In some embodiments, the mixture comprising the complexes and unlinked oligonucleotides comprises trace amounts of phosphate ion. In some embodiments, the mixture comprising the complexes and unlinked oligonucleotides comprises trace amounts of chloride ion. In some embodiments, the mixture comprising the complexes and unlinked oligonucleotides contains trace amounts of both phosphate ion and chloride ion. In some embodiments, the mixture comprising the complexes and unlinked oligonucleotides does not comprise phosphate ion, does not comprise chloride ion, or does not comprise cither phosphate ion or chloride ion. Under these conditions, the unlinked oligonucleotides remain in the flow through and do not adsorb to the mixed-mode resin. In some embodiments, the mixed-mode resin may further be washed between step (i) and step (ii) under these same conditions to remove unlinked oligonucleotides that are loosely bound but not adsorbed to the mixed-mode resin.

In some embodiments, the conditions in step (i) under which the complexes adsorb to the mixed-mode resin also allow some or all (e.g., at least 10%, at least 20%, at least 30%, at least 40%, at least 50%, at least 60%, at least 70%, at least 80%, at least 90%, or 100%) of the unlinked oligonucleotides to adsorb to the mixed-mode resin. In some embodiments, the conditions are achieved by including phosphate ions and/or chloride ions in the mixture in step (i) at a concentration that allows both the complexes and the unlinked oligonucleotides to adsorb to the mixed-mode resin. In some embodiments, the mixture comprising the complexes and unlinked oligonucleotides further comprises up to 5 mM phosphate ion and/or up to 10 mM chloride ions. In some embodiments, the mixture comprising the complexes and unlinked oligonucleotides further comprises up to 5 mM (e.g., up to 5 mM, up to 4 mM, up to 3 mM, up to 2 mM, or up to 1 mM) phosphate ion. Additionally, in some embodiments, the mixture comprising the complexes and unlinked oligonucleotides further comprises up to 10 mM (e.g., up to 10 mM, up to 9 mM, up to 8 mM, up to 7 mM, up to 6 mM, up to 5 mM, up to 4 mM, up to 3 mM, up to 2 mM, or up to 1 mM) chloride ion. In some embodiments, the mixture comprising the complexes and unlinked oligonucleotides further comprises 1-5 mM (e.g., 1-5 mM, 1-4 mM, 1-3 mM, 1-2 mM, 2-5 mM, 2-4 mM, 2-3 mM, 3-5 mM, 3-4 mM, or 4-5 mM) phosphate ion, and/or 1-10 mM (e.g., 1-10 mM, 1-8 mM, 1-6 mM, 1-4 mM, 1-2 mM, 2-10 mM, 2-8 mM, 2-6 mM, 2-4 mM, 4-10 mM, 4-8 mM, 4-6 mM, 6-10 mM, 6-8 mM, or 8-10 mM) chloride ion. In some embodiments, the mixture comprising the complexes and unlinked oligonucleotides further comprises 5 mM, 4 mM, 3 mM, 2 mM, or 1 mM phosphate ion and/or 10 mM, 9 mM, 8 mM, 7 mM, 6 mM, 5 mM, 4 mM, 3 mM, 2 mM, or 1 mM chloride ion. In some embodiments, the mixture comprising the complexes and unlinked oligonucleotides further comprises up to 3 mM phosphate ions and/or up to 8 mM chloride ions. In some embodiments, the mixture comprising the complexes and unlinked oligonucleotides further comprises 1-3 mM (e.g., 1, 2, or 3 mM) phosphate ions and/or 1-8 mM (e.g., 1, 2, 3, 4, 5, 6, 7, or 8 mM) chloride ions. In some embodiments, the mixture comprising the complexes and unlinked oligonucleotides further comprises 3 mM phosphate ions and/or 8 mM chloride ions, e.g., 3 mM phosphate ions and 8 mM chloride ions. In some embodiments, the mixture comprising the complexes and unlinked oligonucleotides comprises trace amounts of phosphate ion. In some embodiments, the mixture comprising the complexes and unlinked oligonucleotides comprises trace amounts of chloride ion. In some embodiments, the mixture comprising the complexes and unlinked oligonucleotides contains trace amounts of both phosphate ion and chloride ion. In some embodiments, the mixture comprising the complexes and unlinked oligonucleotides does not comprise phosphate ion, does not comprise chloride ion, or does not comprise either phosphate ion or chloride ion. Under these conditions, some of all of the unlinked oligonucleotides also adsorb to the mixed-mode resin.

In some embodiments, the mixture comprising the complexes and unlinked oligonucleotides has a pH of 5.0-8.0 (e.g., about 5.0, 5.5, 6.0, 6.5, 7.0, 7.5 or 8.0). In some embodiments, the mixture comprising the complexes and unlinked oligonucleotides has a pH of or about 5.0-6.0 (e.g., about 5.0, 5.1, 5.2, 5.3, 5.4, 5.5, 5.6, 5.7, 5.8, 5.9, or 6.0). In some embodiments, the mixture comprising the complexes and unlinked oligonucleotides has a pH of or about 5.7.

In some embodiments, when some or all of the unlinked oligonucleotides also adsorb to the mix-mode resin, the methods described herein further comprises washing the mixed-mode resin between step (i) and step (ii) with a solution that would dissociate the unlinked oligonucleotides but not the complexes from the mixed-mode resin. In some embodiments, the solution used for washing comprises up to 20 mM phosphate ions and/or up to 30 mM chloride ions, e.g. 20 mM phosphate ion and 30 mM chloride ion. In some embodiments, the solution used for washing comprises up to 20 mM (e.g., up to 20 mM, up to 15 mM, up to 10 mM, or up to 5 mM) phosphate ion. Additionally, in some embodiments, the solution used for washing comprises up to 30 mM (e.g., up to 30 mM, up to 25 mM, up to 20 mM, up to 15 mM, up to 10 mM, or up to 5 mM) chloride ion. In some embodiments, the solution used for washing comprises 5-20 mM (e.g, 5-20 mM, 5-15 mM, 5-10 mM, 10-20 mM, 10-15 mM, or 15-20 mM) phosphate ion and/or 5-30 mM chloride ions (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM). In some embodiments, the solution used for washing comprises 20 mM, 15 mM, 10 mM, 5 mM, or 1 mM phosphate ion and/or 30 mM, 25 mM, 20 mM, 15 mM, 10 mM, or 5 mM chloride ion. In some embodiments, the solution used for washing comprises 20 mM phosphate ion and/or 30 mM chloride ion, e.g., 20 mM phosphate ion and 30 mM chloride ion. In some embodiments, the solution used for washing comprises up to 10 mM phosphate ions and/or up to 25 mM chloride ions, e.g., 10 mM phosphate ions and up to 25 mM chloride ions.

In some embodiments, when some or all of the unlinked oligonucleotides also adsorb to the mix-mode resin, the methods described herein further comprises washing the mixed-mode resin between step (i) and step (ii) with a series of solutions that would dissociate the unlinked oligonucleotides but not the complexes from the mixed-mode resin. In some embodiments, a first solution used for washing comprises 10 mM or about 10 mM phosphate ion and 10 mM or about 10 mM chloride ion. In some embodiments, a second solution for washing comprises 15 mM or about 15 mM phosphate ion and 15 mM or about 15 mM chloride ion. In some embodiments, a third solution for washing comprises 19 mM or about 19 mM phosphate ion and 19 mM or about 19 mM chloride ion. In some embodiments, washing the mixed-mode resin between step (i) and step (ii) comprises washing the resin with a first solution that comprises 10 mM phosphate ion and 10 mM chloride ion, a second solution that comprises 14.5 mM phosphate ion and 14.5 mM chloride ion, and a third solution that comprises 19 mM phosphate ion and 19 mM chloride ion.

In some embodiments, a solution for washing has a pH of 6.0 to 8.5. In some embodiments, a solution for washing has a pH of or about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5. In some embodiments, a solution for washing has a pH of about 6.5.

In some embodiments, to elute the complexes from the mixed-mode resin, in step (ii), the mixed-mode and the bound complexes are subject to conditions that allow the dissociation of the complexes from the mixed-mode resin. In some embodiments, conditions in step (ii) that allow dissociation of the complexes are achieved by applying to the mixed-mode resin an elution solution comprising a higher concentration of phosphate ions and/or chloride ions. The elution step may be done using an elution solution comprising a single phosphate ion concentration, or using an elution solution have a gradient of increasing phosphate ion concentration. Using a gradient of increasing phosphate ion concentration during elution (step (ii)) allows for separation of complexes with different number of drug:antibody ratio (DAR). For example, as the increasing phosphate ion concentration increases in the elution solution, complexes with a lower DAR elutes first, then complexes with higher DAR.

In some embodiments, step (ii) comprises applying an elution solution comprising at least 30 mM phosphate ions and/or at least 50 mM chloride ions to the mixed-mode resin to elute the complexes. In some embodiments, the elution solution comprises at least 30 mM (e.g., at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM) phosphate ions. Additionally, in some embodiments, the elution solution comprises at least 50 mM (e.g., at least 50 mM, at least 60 mM, at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, or at least 200 mM) chloride ions. In some embodiments, the elution solution comprises at least 100 mM phosphate ions and/or at least 100 mM chloride ions. In some embodiments, the elution solution comprises 100 mM phosphate ions and 100 mM chloride ions.

In some embodiments, an elution solution has a pH of 6.0 to 8.5. In some embodiments, an elution solution has a pH of or about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5. In some embodiments, an elution solution has a pH of about 7.5.

In some embodiments, to isolate a complex comprising an anti-TfR Fab covalently linked to a charged oligonucleotide, the reaction mixture containing the complexes and unlinked Fabs and/or oligonucleotides is diluted (e.g., 1:3) in nuclease free water and the pH is adjusted (e.g., to 5.7) with the addition of appropriate buffer (e.g., MES buffer) at an appropriate concentration (e.g., about 50 mM). In some embodiments, the diluted reaction mixture is loaded onto a ceramic hydroxyapatite (HA) column (e.g., at a biomolecule concentration of 8 mg/mL of resin). In some embodiments, the column is washed with wash solution (e.g., 5 mM Na2HPO4, 25 mM NaCl pH 7.0) to remove unlinked oligonucleotide. In some embodiments, the complex comprising anti-TfR Fab linked to oligonucleotide is eluted from the HA column in formulation buffer (e.g., 100 mM Na2HPO4, 100 mM NaCl. pH 7.6). In some embodiments, isolated and purified anti-TfR Fab-oligonucleotide conjugate is analyzed (e.g., by SDS-PAGE and/or analytical SEC) to demonstrate complete removal of unlinked oligonucleotide. In some embodiments, isolated and purified anti-TfR Fab-oligonucleotide conjugate is analyzed by ELISA for human TfR1/cyno TfR1 binding and for endotoxin levels. In some embodiments, the complexes may be alternatively purified by cation exchange and anion exchange.

In some embodiments, the mixtures and solutions used in the method described herein further comprises counter ions for the phosphate ion and/or the chloride ions. In some embodiments, the counter ion for phosphate is a calcium, sodium, magnesium, potassium, or manganese. In some embodiments, the counter ion used in the methods described herein is sodium. In some embodiments, a source of phosphate ions is NaH₂PO₄, Na₂HPO₄, or Na₃PO₄. In some embodiments, the counter ion for chloride is a calcium, sodium, magnesium, potassium, or manganese. In some embodiments, a source of chloride ions is NaCl. One of skill in the art would readily understand that many other equivalent salts and ions may be used for the methods described herein.

In some embodiments, a wash solution and/or an eluent solution may further comprise a buffering agent in order to maintain a consistent pH. In some embodiments, a wash buffer and/or an eluent buffer comprises a neutral pH. In some embodiments, a wash buffer and/or an eluent buffer comprises a pH of about 6, about 6.5, about 7, about 7.5, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

Any mixed-mode resin that comprises positively-charged metal sites and negatively charged ionic sites may be used in accordance with the present disclosure. In some embodiments, the mixed-mode resin used in the methods described herein is an apatite resin. In some embodiments, the apatite resin is a hydroxyapatite resin, a ceramic hydroxyapatite resin, a hydroxyfluoroapatite resin, a fluoroapatite resin, or a chlorapatite resin. An apatite resin may comprise apatite in any form and is typically used as a chromatographic solid phase in the separation and purification of biomolecules, e.g., complexes described herein, using affinity, ion exchange, hydrophobic interactions, or combinations thereof.

In some embodiments, a hydroxyapatite resin is a Bio-Gel HT resin, e.g., from Bio-Rad Laboratories, Inc. (Hercules, Calif., USA). In some embodiments, a ceramic hydroxyapatite resin is a Bio-Scale Mini CHT resin, e.g., from Bio-Rad Laboratories, Inc. In some embodiments, an apatite resin, e.g., ceramic hydroxyapatite, comprises spherical particles of apatite. In some embodiments, the spherical particles of apatite are about 10 microns to about 100 microns, about 25 microns to about 50 microns, about 20 microns, about 30 microns, about 40 microns, about 50 microns, about 60 microns, or about 80 microns in diameter. In some embodiments, an apatite resin, e.g., ceramic hydroxyapatite, is Type I (medium porosity and a high binding capacity) or Type 11 (larger porosity and a lower binding capacity). In some embodiments, an apatite particle may be used in admixture with another separation medium or support.

In some embodiments, a mixed-mode resin may be equilibrated prior to being contacted with a mixture of complex and unlinked oligonucleotide. In some embodiments, a mixed-mode resin is equilibrated using a wash solution, as described above. In some embodiments, a mixed-mode resin is equilibrated to bring the pH of the resin to a neutral pH, a pH of 6-8, a pH of about 6.5, a pH of about 7.0, a pH of about 7.5, or a pH of about 8.0.

In some embodiments, a mixed-mode resin is packed into a column, e.g., a vertical column. In some embodiments, a column may be used under pressure, optionally pressure from top to bottom or bottom to top. In some embodiments, a column may be used without external pressure, e.g., using gravity flow only. In some embodiments, a mixed-mode resin is used as free resin, e.g., using a batch method. In some embodiments, a batch method may further comprise centrifugation and/or filtration steps following contacting of the resin with the mixture of complex and unlinked oligonucleotide.

In some embodiments, the complexes eluted in step (ii) of the mixed-mode resin chromatography described herein comprises an antibody covalently linked to 1, 2, or 3 oligonucleotides. In some embodiments, the complexes having different numbers of linked oligonucleotides (e.g., 1, 2, or 3) are separated in different elution fractions. In some embodiments, the eluent obtained from step (ii) comprises undetectable levels of unlinked oligonucleotide.

C. Compositions of purified complexes

The methods described herein may produce substantially purified complexes, wherein a composition of purified complexes does not comprise detectable quantities of unlinked molecular payloads (e.g., charge-neutral oligonucleotides or charged oligonucleotides) or unlinked antibodies. In some embodiments, a composition of purified complexes comprises a molar or weight ratio of complex:unlinked molecular payloads (e.g., charge-neutral oligonucleotides or charged oligonucleotides) that is at least 9:1, at least 95:5, 96:4, 97:3, 98:2, 99:1, 99.5:0.5, or higher. In some embodiments, a composition of purified complex comprises a molar or weight ratio of complex:unlinked protein that is at least 9:1, at least 95:5, 96:4, 97:3, 98:2, 99:1, 99.5:0.5, or higher.

In some embodiments, a composition of purified complexes does not comprise detectable levels (e.g., detectable quantities) of unlinked protein. In some embodiments, a composition of purified complexes does not comprise detectable levels (e.g., detectable quantities) of unlinked molecular payloads (e.g., charge-neutral oligonucleotides or charged oligonucleotides).

In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked protein (e.g., antibody) by molar ratio. In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked molecular payloads (e.g., charge-neutral oligonucleotides or charged oligonucleotides) by molar ratio. In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked protein (e.g., antibody) by molar ratio, and comprises less than 10%, less than 9%, less than 8%, less than 7%, less than 6%, less than 5%, less than 4%, less than 3%, less than 2%, less than 5%, or less than 0.5% of unlinked molecular payloads (e.g., charge-neutral oligonucleotides or charged oligonucleotides) by molar ratio.

In some embodiments, a composition of purified complexes comprises at least 90% (e.g., at least 90%, at least 91%, at least 92%, at least 93%, at least 94%, at least 95%, at least 96%, at least 97%, at least 98%, at least 99%, at least 99.5%, or at least 99.9%) complexes (i.e., protein (e.g., antibody) covalently linked to one or more oligonucleotides) by molar ratio.

In some embodiments, a composition of purified complexes comprises protein (e.g., antibody) covalently linked to one oligonucleotide, two oligonucleotides, three oligonucleotides and/or more oligonucleotides. In some embodiments, in a composition of purified complexes, at least 50% (e.g., at least 60%, at least 70%, at least 80%, at least 90%, at least 95%) or more of the complexes comprise a protein (e.g., antibody) covalently linked to one oligonucleotide (DAR1). In some embodiments, in a composition of purified complexes, about 50%, 60%, 70%, 80%, 90%, or 95% complexes comprise a protein (e.g., antibody) covalently linked to one oligonucleotide (DAR1).

In some embodiments, in a composition of purified complexes, about 5%, 10%, 15%, 20%, 25%, 30%, or more of complexes comprise a protein (e.g., antibody) covalently linked to two oligonucleotides (DAR2). In some embodiments, in a composition of purified complexes, about 1%, 2%, 3%, 5%, 7%, 10%, 20%, or more of complexes comprise a protein (e.g., antibody) covalently linked to three or more oligonucleotides (DAR3+).

### III. Complexes

In some aspects, provided herein are complexes that comprise a targeting agent, e.g, an antibody, covalently linked to a molecular payload, e.g., an oligonucleotide. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens. A complex may be used to modulate the activity or function of at least one gene, protein, and/or nucleic acid, in some embodiments, the molecular payload present with a complex is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a muscle disease allele in muscle cells.

In some embodiments, a complex comprises a muscle-targeting agent, e.g, an anti-transferrin receptor antibody, covalently linked to a molecular payload, e.g, an antisense oligonucleotide that targets a muscle disease allele.

In some embodiments, a complex is useful for treating a muscle disease, in which a molecular payload affects the activity of the corresponding gene provided in Table 1. For example, depending on the condition, a molecular payload may modulate (e.g., decrease, increase) transcription or expression of the gene, modulate the expression of a protein encoded by the gene, or to modulate the activity of the encoded protein. In some embodiments, the molecular payload is an oligonucleotide that comprises a strand having a region of complementarity to a target gene provided in Table 1.

TABLE 1
List of muscle diseases and corresponding genes.

| | Gene | |
|---|---|---|
| Disease | Symbol | GenBank Accession No. |
| Adult Pompe | GAA | NM_000152; NM_001079803; |
| | | NM_001079804 |
| Adult Pompe | GYS1 | NM_001161587; NM_002103 |
| Centronuclear myopathy (CNM) | DNM2 | NM_001190716; NM_004945; |
| | | NM_001005362; NM_001005360; |
| | | NM_001005361; NM_007871 |
| Duchenne muscular dystrophy | DMD | NM_004023; NM_004020; |
| | | NM_004018; NM_004012 |
| Facioscapulohumeral muscular | DUX4 | NM_001306068; NM_001363820; |
| dystrophy (FSHD) | | NM_001205218; NM_001293798 |
| Familial hypertrophic | MYBPC3 | NM_000256 |
| cardiomyopathy | | |
| Familial hypertrophic | MYH6 | NM_002471; NM_001164171; |
| cardiomyopathy | | NM_010856 |
| Familial hypertrophic | MYH7 | NM_000257; NM_080728 |
| cardiomyopathy | | |
| Familial hypertrophic | TNNI3 | NM_000363 |
| cardiomyopathy | | |
| Familial hypertrophic | TNNT2 | NM_001001432; NM_001001431; |
| cardiomyopathy | | NM_000364; NM_001001430; |
| | | NM_001276347; NM_001276346; |
| | | NM_001276345 |
| Fibrodysplasia Ossificans | ACVR1 | NM_001105; NM_001347663; |
| Progressiva (FOP) | | NM_001347664; NM_001347665; |
| | | NM_001347666; NM_001347667; |
| | | NM_001111067 |
| Friedreich's ataxia (FRDA) | FXN | NM_001161706; NM_181425; |
| | | NM_000144 |
| Inclusion body myopathy 2 | GNE | NM_001190383; NM_001190384; |
| | | NM_001128227; NM_005476; |
| | | NM_001190388 |
| Laing distal myopathy | MYH7 | NM_000257; NM_080728 |
| Myofibrillar myopathy | BAG3 | NM_004281 |
| Myofibrillar myopathy | CRYAB | NM_001885; NM_001330379; |
| | | NM_001289807; NM_001289808 |
| Myofibrillar myopathy | DES | NM_001927 |
| Myofibrillar myopathy | DNAJB6 | NM_005494; NM_058246 |
| Myofibrillar myopathy | FHL1 | NM_001159701; NM_001159699; |
| | | NM_001159702; NM_001159703; |
| | | NM_001159704; NM_001159700; |
| | | NM_001167819; NM_001330659; |
| | | NM_001449; NM_001077362 |
| Myofibrillar myopathy | FLNC | NM_001458; NM_001127487 |
| Myofibrillar myopathy | LDB3 | NM_007078; NM_001171611; |
| | | NM_001171610; NM_001080114; |
| | | NM_001080115; NM_001080116 |
| Myofibrillar myopathy | MYOT | NM_001300911; NM_006790; |
| | | NM_001135940 |
| Myofibrillar myopathy | PLEC | NM_201378; NM_201379; |
| | | NM_201380; NM_201381; |
| | | NM_201382; NM_201383; |
| | | NM_201384; NM_000445 |
| Myofibrillar myopathy | TTN | NM_133432; NM_133379; |
| | | NM_133437; NM_003319; |
| | | NM_001256850; NM_001267550; |
| | | NM_133378 |
| Myotonia congenita (autosomal | CLCN1 | NM_000083; NM_013491 |
| dominant form, Thomsen Disease) | | |
| Myotonic dystrophy type I | DMPK | NM_001081563; NM_004409; |
| | | NM_001081560; NM_001081562; |
| | | NM_001288764; NM_001288765; |
| | | NM_001288766 |
| Myotonic dystrophy type II | CNBP | NM_001127192; NM_001127193; |
| | | NM_001127194; NM_001127195; |
| | | NM_001127196; NM_003418 |
| Myotubular myopathy | MTM1 | NM_000252 |
| Oculopharyngeal muscular dystrophy | PABPN1 | NM_004643 |
| Paramyotonia congenita | SCN4A | NM_000334 |

A. Cell-Targeting Agents

Some aspects of the disclosure provide cell-targeting agents, e.g., muscle-targeting proteins, e.g., for delivering an oligonucleotide to a muscle cell. In some embodiments, such cell-targeting proteins are capable of binding to a specific cell, e.g., via specifically binding to an antigen on said cell, and delivering an associated oligonucleotide to the cell. In some embodiments, the oligonucleotide is bound (e.g., covalently bound) to the cell-targeting agent and is internalized into said cell upon binding of the cell-targeting agent to an antigen on the cell, e.g., via endocytosis.

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. Exemplary muscle-targeting agents are described in further detail herein, however, it should be appreciated that the exemplary muscle-targeting agents provided herein are not meant to be limiting. It should be appreciated that various types of muscle-targeting agents may be used in accordance with the disclosure, and that any muscle targets (e.g., muscle surface proteins) can be targeted by any type of muscle target agents described herein. For example, the muscle-targeting agent may comprise, or consist of, a small molecule, a nucleic acid (e.g., DNA or RNA), a peptide (e.g., an antibody), a lipid (e.g., a microvesicle), or a sugar moiety (e.g., a polysaccharide).

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload (e.g., oligonucleotide) into muscle tissue. Binding to muscle surface recognition elements followed by endocytosis can allow even large molecules such as antibodies to enter muscle cells. As another example oligonucleotides conjugated to transferrin or anti-transferrin receptor antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%. 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

Muscle cells encompassed by the present disclosure include, but are not limited to, skeletal muscle cells, smooth muscle cells, cardiac muscle cells, myoblasts and myocytes.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload (e.g., oligonucleotide), into a muscle cell. As used herein, an antibody that binds to a transferrin receptor may be referred to as an anti-transferrin receptor antibody. Antibodies that bind, e.g. specifically bind, to a transferrin receptor may be internalized into the cell, e.g. through receptor-mediated endocytosis, upon binding to a transferrin receptor.

It should be appreciated that anti-transferrin receptor antibodies may be produced, synthesized, and/or derivatized using several known methodologies, e.g. library design using phage display. Exemplary methodologies have been characterized in the art and are incorporated by reference (Díez, P. et al. “High-throughput phage-display screening in array format”, Enzyme and microbial technology, 2015, 79, 34-41; Christoph M. H, and Stanley, J. R. “Antibody Phage Display: Technique and Applications” J Invest Dermatol. 2014, 134:2; Engleman, Edgar (Ed.) “Human Hybridomas and Monoclonal Antibodies.” 1985, Springer). In other embodiments, an anti-transferrin antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052).

Any appropriate anti-transferrin receptor antibodies may be used in the complexes disclosed herein. Examples of anti-transferrin receptor antibodies, including associated references and binding epitopes, are listed in Table 2. In some embodiments, the anti-transferrin receptor antibody comprises the complementarity determining regions (CDR-Hi, CDR-H2, CDR-H3, CDR-L1, CDR-L2, and CDR-L3) of any of the anti-transferrin receptor antibodies provided herein, e.g., anti-transferrin receptor antibodies listed in Table 2.

TABLE 2
List of anti-transferrin receptor antibody clones, including
associated references and binding epitope information.

| Antibody | | |
|---|---|---|
| Clone Name | Reference(s) | Epitope/Notes |
| OKT9 | U.S. Pat. No. 4,364,934, filed Dec. 4, 1979, | Apical domain of TfR |
| | entitled “MONOCLONAL ANTIBODY | (residues 305-366 of |
| | TO A HUMAN EARLY THYMOCYTE | human TfR sequence |
| | ANTIGEN AND METHODS FOR | XM_052730.3, |
| | PREPARING SAME” | available in GenBank) |
| | Schneider C. et al. “Structural features of | |
| | the cell surface receptor for transferrin that | |
| | is recognized by the monoclonal antibody | |
| | OKT9.” J Biol Chem. 1982, 257: 14, 8516-8522. | |
| (From JCR) | WO 2015/098989, filed | Apical domain |
| Clone M11 | Dec. 24, 2014, “Novel anti-Transferrin | (residues 230-244 and |
| Clone M23 | receptor antibody that passes through | 326-347 of TfR) and |
| Clone M27 | blood-brain barrier” | protease-like domain |
| Clone B84 | U.S. Pat. No. 9,994,641, filed | (residues 461-473) |
| | Dec. 24, 2014, “Novel anti-Transferrin | |
| | receptor antibody that passes through | |
| | blood-brain barrier” | |
| (From | WO 2016/081643, filed May 26, 2016, | Apical domain and |
| Genentech) | entitled “ANTI-TRANSFERRIN | non-apical regions |
| 7A4, 8A2, 15D2, | RECEPTOR ANTIBODIES AND | |
| 10D11, 7B10, 15G11, | METHODS OF USE” | |
| 16G5, 13C3, 16G4, | U.S. Pat. No. 9,708,406, filed | |
| 16F6, 7G7, 4C2, | May, 20, 2014, “Anti-transferrin receptor | |
| 1B12, and 13D4 | antibodies and methods of use” | |
| (From | Lee et al. “Targeting Rat Anti- | |
| Armagen) | Mouse Transferrin Receptor Monoclonal | |
| 8D3 | Antibodies through Blood-Brain Barrier in | |
| | Mouse” 2000, J Pharmacol. Exp. Ther., | |
| | 292: 1048-1052. | |
| | U.S. patent application 2010/077498, filed | |
| | Sep. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute | |
| | to autophagosome formation in response to | |
| | Group A Streptococcus invasion. | |
| | Cellular microbiology. 16: 1806-21. | |
| DF1513 | Ortiz-Zapater E et al. Trafficking | |
| | of the human transferrin receptor in plant | |
| | cells: effects of tyrphostin A23 and | |
| | brefeldin A. Plant J 48: 757-70 (2006). | |
| 1A1B2, 66IG10, | Commercially available anti- | Novus Biologicals |
| MEM-189, JF0956, | transferrin receptor antibodies. | 8100 Southpark Way, |
| 29806, 1A1B2, | | A-8 Littleton CO |
| TFRC/1818, 1E6, | | 80120 |
| 66Ig10, TFRC/1059, | | |
| Q1/71, 23D10, | | |
| 13E4, TFRC/1149, | | |
| ER-MP21, YTA74.4, | | |
| BU54, 2B6, RI7 217 | | |
| (From | U.S. patent application 2011/0311544A1, | Does not compete |
| INSERM) | filed Jun. 15, 2005, entitled “ANTI-CD71 | with OKT9 |
| BA120g | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 cpitopc” |
| | Jul. 7, 2004, entitled “TRANSFERRIN | |
| | RECEPTOR ANTIBODIES” | |
| (Salk Institute) | Trowbridge, I.S. et al. “Anti-transferrin | |
| B3/25 | receptor monoclonal antibody and | |
| T58/30 | toxin-antibody conjugates affect | |
| | growth of human tumour cells.” | |
| | Nature, 1981, volume 294, pages 171-173 | |
| R17 217.1.3, | Commercially available anti- | BioXcell |
| 5E9C11, OKT9 | transferrin receptor antibodies. | 10 Technology Dr., |
| (BE0023 | | Suite 2B |
| clone) | | West Lebanon, NH |
| | | 03784-1671 USA |
| BK19.9, | Gatter, K.C. et al. “Transferrin | |
| B3/25, T56/14 | receptors in human tissues: their | |
| and T58/1 | distribution and possible clinical | |
| | relevance.” J Clin Pathol. 1983 | |
| | May; 36(5): 539-45. | |

In some embodiments, the muscle-targeting agent is an anti-transferrin receptor antibody. In some embodiment, an anti-transferrin receptor antibody specifically binds to a transferrin protein having an amino acid sequence as disclosed herein. In some embodiments, an anti-transferrin receptor antibody may specifically bind to any extracellular epitope of a transferrin receptor or an epitope that becomes exposed to an antibody, including the apical domain, the transferrin binding domain, and the protease-like domain. In some embodiments, an anti-transferrin receptor antibody binds to an amino acid segment of a human or non-human primate transferrin receptor, as provided in SEQ ID NOs. 1-3 in the range of amino acids C89 to F760. In some embodiments, an anti-transferrin receptor antibody specifically binds with binding affinity of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. Anti-transferrin receptor antibodies used herein may be capable of competing for binding with other anti-transferrin receptor antibodies, e.g. OKT9, 8D3, that bind to transferrin receptor with 10⁻³ M, 10⁻⁴ M, 10⁻⁵ M, 10⁻⁶ M, 10⁻⁷ M, or less.

An example human transferrin receptor amino acid sequence, corresponding to NCBI sequence NP_003225.2 (transferrin receptor protein 1 isoform 1, Homo sapiens) is as follows:

(SEQ ID NO: 1)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 2)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 3)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYTENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 4)
MMDQARSAFSNLFGGEPLSYTRFSLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLQWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF

In some embodiments, an anti-transferrin receptor antibody binds to an amino acid segment of the receptor as follows: FVKIQVKDSAQNSVIIVDKNGRLVYLVENPGGYVAYSKAATVTGKLVHANFGTKKDF EDLYTPVNGSIVIVRAGKITFAEKVANAESLNAIGVLIYMDQTKFPIVNAELSFFGHAH LGTGDPYTPGFPSFNHTQFPPSRSSGLPNIPVQTTSRAAAEKLFGNMEGDCPSDWKTDS TCRMVTSESKNVKLTVSNVLKE (SEQ ID NO: 5) and does not inhibit the binding interactions between transferrin receptors and transferrin and/or human hemochromatosis protein (also known as HFE).

Appropriate methodologies may be used to obtain and/or produce antibodies, antibody fragments, or antigen-binding agents, e.g., through the use of recombinant DNA protocols. In some embodiments, an antibody may also be produced through the generation of hybridomas (sec, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see. e.g. Harlow et al. “Antibodies: A Laboratory Manual”, Cold Spring Harbor Laboratory, 1988).

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g, a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

Some aspects of the disclosure provide proteins that bind to transferrin receptor (e.g., an extracellular portion of the transferrin receptor). In some embodiments, transferrin receptor antibodies provided herein bind specifically to transferrin receptor (e.g., human transferrin receptor). Transferrin receptors are internalizing cell surface receptors that transport transferrin across the cellular membrane and participate in the regulation and homeostasis of intracellular iron levels. In some embodiments, transferrin receptor antibodies provided herein bind specifically to transferrin receptor from human, non-human primates, mouse, rat, etc. In some embodiments, transferrin receptor antibodies provided herein bind to human transferrin receptor. In some embodiments, transferrin receptor antibodies provided herein specifically bind to human transferrin receptor. In some embodiments, transferrin receptor antibodies provided herein bind to an apical domain of human transferrin receptor. In some embodiments, transferrin receptor antibodies provided herein specifically bind to an apical domain of human transferrin receptor.

In some embodiments, transferrin receptor antibodies of the present disclosure include one or more of the CDR-H (e.g., CDR-H1, CDR-H2, and CDR-H3) amino acid sequences from any one of the anti-transferrin receptor antibodies selected from Table 2. In some embodiments, transferrin receptor antibodies include the CDR-H1, CDR-H2, and CDR-H3 as provided for any one of the anti-transferrin receptor antibodies selected from Table 2. In some embodiments, anti-transferrin receptor antibodies include the CDR-L1, CDR-L2, and CDR-L3 as provided for any one of the anti-transferrin receptor antibodies selected from Table 2. In some embodiments, anti-transferrin antibodies include the CDR-H1, CDR-H2, CDR-H3, CDR-L1, CDR-L2, and CDR-L3 as provided for any one of the anti-transferrin receptor antibodies selected from Table 2. The disclosure also includes any nucleic acid sequence that encodes a molecule comprising a CDR-H1, CDR-H2, CDR-H3, CDR-L1, CDR-L2, or CDR-L3 as provided for any one of the anti-transferrin receptor antibodies selected from Table 2. In some embodiments, antibody heavy and light chain CDR3 domains may play a particularly important role in the binding specificity/affinity of an antibody for an antigen. Accordingly, anti-transferrin receptor antibodies of the disclosure may include at least the heavy and/or light chain CDR3s of any one of the anti-transferrin receptor antibodies selected from Table 2.

In some examples, any of the anti-transferrin receptor antibodies of the disclosure have one or more CDR (e.g., CDR-H or CDR-L) sequences substantially similar to any of the CDR-H1, CDR-H2, CDR-H3, CDR-L1, CDR-L2, and/or CDR-L3 sequences from one of the anti-transferrin receptor antibodies selected from Table 2. In some embodiments, the position of one or more CDRs along the VH (e.g., CDR-H1, CDR-H2, or CDR-H3) and/or VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1. CDR-H2, or CDR-H3) and/or VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or CDR-H3 described herein may be one, two, three, four, five or more amino acids shorter than one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or CDR-H3 described herein may be one, two, three, four, five or more amino acids longer than one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-transferrin receptor antibodies of the disclosure have one or more CDR (e.g., CDR-H or CDR-L) sequences substantially similar to any one of the anti-transferrin receptor antibodies selected from Table 2. For example, the antibodies may include one or more CDR sequence(s) from any of the anti-transferrin receptor antibodies selected from Table 2 containing up to 5, 4, 3, 2, or 1 amino acid residue variations as compared to the corresponding CDR region in any one of the CDRs provided herein (e.g., CDRs from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide transferrin receptor antibodies that comprise one or more of the heavy chain variable (VH) and/or light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-transferrin receptor antibodies selected from Table 2. In some embodiments, any of the VL domains provided herein include one or more of the CDR-L sequences (e.g., CDR-L1, CDR-L2, and CDR-L3) provided herein, for example, any of the CDR-L sequences provided in any one of the anti-transferrin receptor antibodies selected from Table 2.

In some embodiments, anti-transferrin receptor antibodies of the disclosure include any antibody that includes a heavy chain variable domain and/or a light chain variable domain of any anti-transferrin receptor antibody, such as any one of the anti-transferrin receptor antibodies selected from Table 2. In some embodiments, anti-transferrin receptor antibodies of the disclosure include any antibody that includes the heavy chain variable and light chain variable pairs of any anti-transferrin receptor antibody, such as any one of the anti-transferrin receptor antibodies selected from Table 2.

Aspects of the disclosure provide anti-transferrin receptor antibodies having a heavy chain variable (VH) and/or a light chain variable (VL) domain amino acid sequence homologous to any of those described herein. In some embodiments, the anti-transferrin receptor antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-transferrin receptor antibody, such as any one of the anti-transferrin receptor antibodies selected from Table 2. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-transferrin receptor antibodies provided herein comprise a heavy chain variable sequence and a light chain variable sequence that comprises a framework sequence that is at least 75%, 80%, 85%, 90%, 95%, 98%, or 99% identical to the framework sequence of any anti-transferrin receptor antibody, such as any one of the anti-transferrin receptor antibodies selected from Table 2.

In some embodiments, an anti-transferrin receptor antibody, which specifically binds to transferrin receptor (e.g., human transferrin receptor), comprises a light chain variable VL domain comprising any of the CDR-L domains (CDR-L1, CDR-L2, and CDR-L3), or CDR-L domain variants provided herein, of any of the anti-transferrin receptor antibodies selected from Table 2. In some embodiments, an anti-transferrin receptor antibody, which specifically binds to transferrin receptor (e.g., human transferrin receptor), comprises a light chain variable VL domain comprising the CDR-L1, the CDR-L2, and the CDR-L3 of any anti-transferrin receptor antibody, such as any one of the anti-transferrin receptor antibodies selected from Table 2. In some embodiments, the anti-transferrin receptor antibody comprises a light chain variable (VL) region sequence comprising one, two, three or four of the framework regions of the light chain variable region sequence of any anti-transferrin receptor antibody, such as any one of the anti-transferrin receptor antibodies selected from Table 2. In some embodiments, the anti-transferrin receptor antibody comprises one, two, three or four of the framework regions of a light chain variable region sequence which is at least 75%, 80%, 85%, 90%, 95%, or 100% identical to one, two, three or four of the framework regions of the light chain variable region sequence of any anti-transferrin receptor antibody, such as any one of the anti-transferrin receptor antibodies selected from Table 2. In some embodiments, the light chain variable framework region that is derived from said amino acid sequence consists of said amino acid sequence but for the presence of up to 10 amino acid substitutions, deletions, and/or insertions, preferably up to 10 amino acid substitutions. In some embodiments, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region.

In some embodiments, an anti-transferrin receptor antibody that specifically binds to transferrin receptor comprises the CDR-L1, the CDR-L2, and the CDR-L3 of any anti-transferrin receptor antibody, such as any one of the anti-transferrin receptor antibodies selected from Table 2. In some embodiments, the antibody further comprises one, two, three or all four VL framework regions derived from the VL of a human or primate antibody. The primate or human light chain framework region of the antibody selected for use with the light chain CDR sequences described herein, can have, for example, at least 70% (e.g., at least 75%, 80%, 85%, 90%, 95%, 98%, or at least 99%) identity with a light chain framework region of a non-human parent antibody. The primate or human antibody selected can have the same or substantially the same number of amino acids in its light chain complementarity determining regions to that of the light chain complementarity determining regions of any of the antibodies provided herein, e.g., any of the anti-transferrin receptor antibodies selected from Table 2. In some embodiments, the primate or human light chain framework region amino acid residues are from a natural primate or human antibody light chain framework region having at least 75% identity, at least 80% identity, at least 85% identity, at least 90% identity, at least 95% identity, at least 98% identity, at least 99% (or more) identity with the light chain framework regions of any anti-transferrin receptor antibody, such as any one of the anti-transferrin receptor antibodies selected from Table 2. In some embodiments, an anti-transferrin receptor antibody further comprises one, two, three or all four VL framework regions derived from a human light chain variable kappa subfamily. In some embodiments, an anti-transferrin receptor antibody further comprises one, two, three or all four VL framework regions derived from a human light chain variable lambda subfamily.

In some embodiments, any of the anti-transferrin receptor antibodies provided herein comprise a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. It should be appreciated that any of the light chain constant regions provided herein may be variants of any of the light chain constant regions provided herein. In some embodiments, the light chain constant region comprises an amino acid sequence that is at least 75%, 80%, 85%, 90%, 95%, 98%, or 99% identical to any of the light chain constant regions of any anti-transferrin receptor antibody, such as any one of the anti-transferrin receptor antibodies selected from Table 2.

In some embodiments, the anti-transferrin receptor antibody is any anti-transferrin receptor antibody, such as any one of the anti-transferrin receptor antibodies selected from Table 2.

In some embodiments, an anti-transferrin receptor antibody comprises a VL domain comprising the amino acid sequence of any anti-transferrin receptor antibody, such as any one of the anti-transferrin receptor antibodies selected from Table 2, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In some embodiments, an anti-transferrin receptor antibody comprises any of the VL domains, or VL domain variants, and any of the VH domains, or VH domain variants, wherein the VL and VH domains, or variants thereof, are from the same antibody clone, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kahat E A et al., (1991) supra.

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between 5 pM and 500 nM, e.g., between 50 pM and 100 nM, e.g., between 500 pM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 pM or lower, 50 pM or lower, or 5 pM or lower). The affinity and binding kinetics of the anti-transferrin receptor antibody can be tested using any suitable method including but not limited to biosensor technology (e.g., OCTET or BIACORE).

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between 5 pM and 500 nM, e.g., between 50 pM and 100 nM, e.g., between 500 pM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower. 10 nM or lower, 500 pM or lower, 50 pM or lower, or 5 pM or lower). The affinity and binding kinetics of the anti-transferrin receptor antibody can be tested using any suitable method including but not limited to biosensor technology (e.g., OCTET or BIACORE).

In some embodiments, the muscle-targeting agent is a transferrin receptor antibody (e.g., the antibody and variants thereof as described in International Application Publication WO 2016/081643, incorporated herein by reference).

The heavy chain and light chain CDRs of the antibody according to different definition systems are provided in Table 3. The different definition systems, e.g., the Kabat definition, the Chothia definition, and/or the contact definition have been described. See, e.g., (e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877: Chothia. C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

TABLE 3
Heavy chain and light chain CDRs of a mouse transferrin receptor antibody.

| CDRs | Kabat | Chothia | Contact |
|---|---|---|---|
| CDR-H1 | SYWMH | GYTFTSY | TSYWMH |
| | (SEQ ID NO: 17) | (SEQ ID NO: 23) | (SEQ ID NO: 25) |
| CDR-H2 | EINPTNGRTNYIEKFKS | NPTNGR | WIGEINPTNGRTN |
| | (SEQ ID NO: 18) | (SEQ ID NO: 24) | (SEQ ID NO: 26) |
| CDR-H3 | GTRAYHY | GTRAYHY | ARGTRA |
| | (SEQ ID NO: 19) | (SEQ ID NO: 19) | (SEQ ID NO: 27) |
| CDR-L1 | RASDNLYSNLA | RASDNLYSNLA | YSNLAWY |
| | (SEQ ID NO: 20) | (SEQ ID NO: 20) | (SEQ ID NO: 28) |
| CDR-L2 | DATNLAD | DATNLAD | LLVYDATNLA |
| | (SEQ ID NO: 21) | (SEQ ID NO: 21) | (SEQ ID NO: 29) |
| CDR-L3 | QHFWGTPLT | QHFWGTPLT | QHFWGTPL |
| | (SEQ ID NO: 22) | (SEQ ID NO: 22) | (SEQ ID NO: 30) |

The heavy chain variable domain (VH) and light chain variable domain sequences are also provided:

VH
(SEQ ID NO: 33)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSS
VL
(SEQ ID NO: 34)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELK

In some embodiments, the transferrin receptor antibody of the present disclosure comprises a CDR-H1, a CDR-H2, and a CDR-H3 that are the same as the CDR-H1, CDR-H2, and CDR-H3 shown in Table 3. Alternatively or in addition, the transferrin receptor antibody of the present disclosure comprises a CDR-L1, a CDR-L2, and a CDR-L3 that are the same as the CDR-L1, CDR-L2, and CDR-L3 shown in Table 3.

In some embodiments, the transferrin receptor antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1, CDR-H2, and CDR-H3 as shown in Table 3. “Collectively” means that the total number of amino acid variations in all of the three heavy chain CDRs is within the defined range. Alternatively or in addition, the transferrin receptor antibody of the present disclosure may comprise a CDR-L1, a CDR-L2, and a CDR-L3, which collectively contains no more than 5 amino acid variations (e.g., no more than 5, 4, 3, 2 or 1 amino acid variation) as compared with the CDR-L1, CDR-L2, and CDR-L3 as shown in Table 3.

In some embodiments, the transferrin receptor antibody of the present disclosure comprises a CDR-H1, a CDR-H2, and a CDR-H3, at least one of which contains no more than 3 amino acid variations (e.g., no more than 3, 2, or 1 amino acid variation) as compared with the counterpart heavy chain CDR as shown in Table 3. Alternatively or in addition, the transferrin receptor antibody of the present disclosure may comprise CDR-L1, a CDR-L2, and a CDR-L3, at least one of which contains no more than 3 amino acid variations (e.g., no more than 3, 2, or 1 amino acid variation) as compared with the counterpart light chain CDR as shown in Table 3.

In some embodiments, the transferrin receptor antibody of the present disclosure comprises a CDR-L3, which contains no more than 3 amino acid variations (e.g., no more than 3, 2, or 1 amino acid variation) as compared with the CDR-L3 as shown in Table 3. In some embodiments, the transferrin receptor antibody of the present disclosure comprises a CDR-L3 containing one amino acid variation as compared with the CDR-L3 as shown in Table 3. In some embodiments, the transferrin receptor antibody of the present disclosure comprises a CDR-L3 of QHFAGTPLT (SEQ ID NO: 31 according to the Kabat and Chothia definition system) or QHFAGTPL (SEQ ID NO: 32 according to the Contact definition system). In some embodiments, the transferrin receptor antibody of the present disclosure comprises a CDR-H1, a CDR-H2, a CDR-H3, a CDR-L1 and a CDR-L2 that are the same as the CDR-H1, CDR-H2, and CDR-H3 shown in Table 3, and comprises a CDR-L3 of QHFAGTPLT (SEQ ID NO: 31 according to the Kabat and Chothia definition system) or QHFAGTPL (SEQ ID NO: 32 according to the Contact definition system).

In some embodiments, the transferrin receptor antibody of the present disclosure comprises heavy chain CDRs that collectively are at least 80% (e.g., 80%, 85%, 90%, 95%, or 98%) identical to the heavy chain CDRs as shown in Table 3. Alternatively or in addition, the transferrin receptor antibody of the present disclosure comprises light chain CDRs that collectively are at least 80% (e.g., 80%, 85%, 90%, 95%, or 98%) identical to the light chain CDRs as shown in Table 3.

In some embodiments, the transferrin receptor antibody of the present disclosure comprises a VH comprising the amino acid sequence of SEQ ID NO: 33. Alternatively or in addition, the transferrin receptor antibody of the present disclosure comprises a VL comprising the amino acid sequence of SEQ ID NO: 34.

In some embodiments, the transferrin receptor antibody of the present disclosure comprises a VII containing no more than 20 amino acid variations (e.g., no more than 20, 19, 18, 17, 16, 15, 14, 13, 12, 11, 10, 9, 8.7, 6, 5, 4, 3, 2, or 1 amino acid variation) as compared with the VH as set forth in SEQ ID NO: 33. Alternatively or in addition, the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 34.

In some embodiments, the transferrin receptor antibody of the present disclosure comprises a VII comprising an amino acid sequence that is at least 80% (e.g., 80%, 85%, 90%, 95%, or 98%) identical to the VII as set forth in SEQ ID NO: 33. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 34.

In some embodiments, the transferrin receptor antibody of the present disclosure is a humanized antibody (e.g., a humanized variant of an antibody). In some embodiments, the transferrin receptor antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 3, and comprises a humanized heavy chain variable region and/or a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 3) into the IGKV1-NL1*01 and 1GHV1-3*01 human variable domains. In some embodiments, the transferrin receptor antibody of the present disclosure is a humanized variant comprising one or more amino acid substitutions at positions 9, 13, 17, 18, 40, 45, and 70 as compared with the VL as set forth in SEQ ID NO: 34, and/or one or more amino acid substitutions at positions 1, 5, 7, 11, 12, 20, 38, 40, 44, 66, 75, 81, 83, 87, and 108 as compared with the VH as set forth in SEQ ID NO: 33. In some embodiments, the transferrin receptor antibody of the present disclosure is a humanized variant comprising amino acid substitutions at all of positions 9, 13, 17, 18, 40, 45, and 70 as compared with the VL as set forth in SEQ ID NO: 34, and/or amino acid substitutions at all of positions 1, 5, 7, 11, 12, 20, 38, 40, 44, 66, 75, 81, 83, 87, and 108 as compared with the VH as set forth in SEQ ID NO: 33.

In some embodiments, the transferrin receptor antibody of the present disclosure is a humanized antibody and contains the residues at positions 43 and 48 of the VL as set forth in SEQ ID NO: 34. Alternatively or in addition, the transferrin receptor antibody of the present disclosure is a humanized antibody and contains the residues at positions 48, 67, 69, 71, and 73 of the VH as set forth in SEQ ID NO: 33.

The VH and VL amino acid sequences of an example humanized antibody that may be used in accordance with the present disclosure are provided:

Humanized VH
(SEQ ID NO: 35)
EVQLVQSGAEVKKPGASVKVSCKASGYTFTSYWMHWVRQAPGQRLEWIG
EINPTNGRTNYTEKFKSRATLTVDKSASTAYMELSSLRSEDTAVYYCAR
GTRAYHYWGQGTMVTVSS
Humanized VL
(SEQ ID NO: 36)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIK

In some embodiments, the transferrin receptor antibody of the present disclosure comprises a VH comprising the amino acid sequence of SEQ ID NO: 35. Alternatively or in addition, the transferrin receptor antibody of the present disclosure comprises a VL comprising the amino acid sequence of SEQ ID NO: 36.

In some embodiments, the transferrin receptor antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 35. Alternatively or in addition, the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 36.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 35. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 36.

In some embodiments, the transferrin receptor antibody of the present disclosure is a humanized variant comprising amino acid substitutions at one or more of positions 43 and 48 as compared with the VL as set forth in SEQ ID NO: 34, and/or amino acid substitutions at one or more of positions 48, 67, 69, 71, and 73 as compared with the VH as set forth in SEQ ID NO: 33. In some embodiments, the transferrin receptor antibody of the present disclosure is a humanized variant comprising a S43A and/or a V48L mutation as compared with the VL as set forth in SEQ ID NO: 34, and/or one or more of A67V, L69I, V71R, and K73T mutations as compared with the VH as set forth in SEQ ID NO: 33

In some embodiments, the transferrin receptor antibody of the present disclosure is a humanized variant comprising amino acid substitutions at one or more of positions 9, 13, 17, 18, 40, 43, 48, 45, and 70 as compared with the VL as set forth in SEQ ID NO: 34, and/or amino acid substitutions at one or more of positions 1, 5, 7, 11, 12, 20, 38, 40, 44, 48, 66, 67, 69, 71, 73, 75, 81, 83, 87, and 108 as compared with the VH as set forth in SEQ ID NO: 33.

In some embodiments, the transferrin receptor antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the transferrin receptor antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the heavy chain of any of the transferrin receptor antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An exemplary human IgG1 constant region is given below:

(SEQ ID NO: 37)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the transferrin receptor antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 38)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php., both of which are incorporated by reference herein.

Exemplary heavy chain and light chain amino acid sequences of the transferrin receptor antibodies described are provided below:

Heavy Chain (VH + human IgG1 constant region)
(SEQ ID NO: 39)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (VL + kappa light chain)
(SEQ ID NO: 40)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Heavy Chain (humanized VH + human IgG1 constant
region)
(SEQ ID NO: 41)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (humanized VL + kappa light chain)
(SEQ ID NO: 42)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the transferrin receptor antibody described herein comprises a heavy chain comprising an amino acid sequence that is at least 80% (e.g., 80%, 85%, 90%, 95%, or 98%) identical to SEQ ID NO: 39. Alternatively or in addition, the transferrin receptor antibody described herein comprises a light chain comprising an amino acid sequence that is at least 80% (e.g., 80%, 85%, 90%, 95%, or 98%) identical to SEQ ID NO: 40. In some embodiments, the transferrin receptor antibody described herein comprises a heavy chain comprising the amino acid sequence of SEQ ID NO: 39. Alternatively or in addition, the transferrin receptor antibody described herein comprises a light chain comprising the amino acid sequence of SEQ ID NO: 40.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 40.

In some embodiments, the transferrin receptor antibody described herein comprises a heavy chain comprising an amino acid sequence that is at least 80% (e.g., 80%, 85%, 90%, 95%, or 98%) identical to SEQ ID NO: 41. Alternatively or in addition, the transferrin receptor antibody described herein comprises a light chain comprising an amino acid sequence that is at least 80% (e.g., 80%, 85%, 90%, 95%, or 98%) identical to SEQ ID NO: 42. In some embodiments, the transferrin receptor antibody described herein comprises a heavy chain comprising the amino acid sequence of SEQ ID NO: 41. Alternatively or in addition, the transferrin receptor antibody described herein comprises a light chain comprising the amino acid sequence of SEQ ID NO: 42.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain of humanized antibody as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain of humanized antibody as set forth in SEQ ID NO: 40.

In some embodiments, the transferrin receptor antibody is an antigen binding fragment (Fab) of an intact antibody (full-length antibody). Antigen binding fragment of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Exemplary Fab's amino acid sequences of the transferrin receptor antibodies described herein are provided below:

Heavy Chain FAB (VH + a portion of human IgG1
constant region)
(SEQ ID NO: 43)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP
Heavy Chain FAB (humanized VH + a portion of
human IgG1 constant region)
(SEQ ID NO: 44)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP

The transferrin receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the transferrin receptor antibody described herein is a scFv. In some embodiments, the transferrin receptor antibody described herein is a scFv-Fab (e.g., scFv fused to a portion of a constant region). In some embodiments, the transferrin receptor antibody described herein is a scFv fused to a constant region (e.g., human IgG1 constant region as set forth in SEQ ID NO: 39).

b. Other Muscle-Targeting Antibodies

In some embodiments, the muscle-targeting antibody is an antibody that specifically binds hemojuvelin, caveolin-3. Duchenne muscular dystrophy peptide, myosin lib or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1. Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as scrum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. Sec, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the Cl component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g, a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M, et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 6) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 6). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 7) was identified and this muscle-targeting peptide showed improved binding to C2C12 cells relative to the ASSLNIA (SEQ ID NO: 6) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 8) appeared most frequently. Accordingly, in some embodiments, the muscle-targeting agent comprises the amino acid sequence TARGEHKEEELI (SEQ ID NO: 8).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P, and Brown, K. C. “Combinatorial Peptide Libraries: Mining for Cell-Binding Peptides” Chem Rev. 2014, 114:2, 1020-1081; Samoylova, T. I, and Smith, B. F. “Elucidation of muscle-binding peptides by phage display screening.” Muscle Nerve, 1999, 22:4, 460-6). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7; Zhang, L. “Molecular profiling of heart endothelial cells.” Circulation, 2005, 112:11, 1601-11; McGuire, M J. et al. “In vitro selection of a peptide with high selectivity for cardiomyocytes in vivo.” J Mol Biol. 2004, 342:1, 171-82). Exemplary muscle-targeting peptides comprise an amino acid sequence of the following group: CQAQGQLVC (SEQ ID NO: 9), CSERSMNFC (SEQ ID NO: 10), CPKTRRVPC (SEQ ID NO: 11), WLSEAGPVVTVRALRGTGSW (SEQ ID NO: 12), ASSLNIA (SEQ ID NO: 6), CMQHSMRVC (SEQ ID NO: 13), and DDTRHWG (SEQ ID NO: 14). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147). A muscle-targeting agent may be an aptamer, e.g, a peptide aptamer, which preferentially targets muscle cells relative to other cell types.

iii. Muscle-Targeting Receptor Ligands

A muscle-targeting agent may be a ligand, e.g, a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g, a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agents described herein (e.g., antibodies, nucleic acids, small molecules, peptides, aptamers, lipids, sugar moieties) that target SLC superfamily of transporters. In some embodiments, the muscle-targeting agent may target the transporter (e.g., may be a substrate). SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/camitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the camitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or lack a C-terminal anchoring domain. In some embodiments, hemojuvelin may be annotated under GenBank RefSeq Accession Numbers NM_001316767.1, NM_145277.4, NM 202004.3. NM_213652.3, or NM 213653.3. It should be appreciated that a hemojuvelin may be of human, non-human primate, or rodent origin.

B. Molecular Payloads

Some aspects of the disclosure provide molecular payloads, e.g., for modulating a biological outcome, e.g., the transcription of a DNA sequence, the expression of a protein, or the activity of a protein. In some embodiments, a molecular payload is covalently linked to, or otherwise associated with a muscle-targeting agent. It should be appreciated that various types of muscle-targeting agents may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, such molecular payloads are capable of targeting to a muscle cell, e.g., via specifically binding to a nucleic acid or protein in the muscle cell following delivery to the muscle cell by an associated muscle-targeting agent. In some embodiments, the molecular payload is an oligonucleotide that comprises a strand having a region of complementarity to a gene provided in Table 1. Exemplary molecular payloads are described in further detail herein, however, it should be appreciated that the exemplary molecular payloads provided herein are not meant to be limiting.

In some embodiments at least one (e.g., at least 2, at least 3, at least 4, at least 5, at least 10) molecular payload (e.g., oligonucleotides) is covalently linked to a muscle-targeting agent. In some embodiments, all molecular payloads attached to a muscle-targeting agent are the same, e.g. target the same gene. In some embodiments, all molecular payloads attached to a muscle-targeting agent are different, for example the molecular payloads may target different portions of the same target gene, or the molecular payloads may target at least two different target genes. In some embodiments, a muscle-targeting agent may be attached to some molecular payloads that are the same and some molecular payloads that are different.

The present disclosure also provides a composition comprising a plurality of complexes, for which at least 80% (e.g., at least 85%, at least 90%, at least 91%, at least 92%, at least 93%, at least 94%, at least 95%, at least 96%, at least 97%, at least 98%, or at least 99%) of the complexes comprise a muscle-targeting agent covalently linked to the same number of molecular payloads (e.g., oligonucleotides).

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

In some embodiments, an oligonucleotide may comprise a region of complementarity to a target gene provided in Table 1. Further non-limiting examples are provided below for selected genes of Table 1.

DMPK/DM1

In some embodiments, examples of oligonucleotides useful for targeting DMPK, e.g., for the treatment of DM1, are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; Pandey, S. K. et al. “Identification and Characterization of Modified Antisense Oligonucleotides Targeting DMPK in Mice and Nonhuman Primates for the Treatment of Myotonic Dystrophy Type 1” J. of Pharmacol Exp Ther, 2015, 355:329-340; Langlois, M. et al. “Cytoplasmic and Nuclear Retained DMPK mRNAs Are Targets for RNA Interference in Myotonic Dystrophy Cells” J. Biological Chemistry, 2005, 280:17, 16949-16954; Jauvin, D. et al. “Targeting DMPK with Antisense Oligonucleotide Improves Muscle Strength in Myotonic Dystrophy Type 1 Mice”, Mol. Ther. Nucleic Acids, 2017, 7:465-474; Mulders, S. A. et al. “Triplet-repeat oligonucleotide-mediated reversal of RNA toxicity in myotonic dystrophy” PNAS, 2009, 106:33, 13915-13920; Wheeler, T. M. et al., “Targeting nuclear RNA for in vivo correction of myotonic dystrophy” Nature, 2012, 488(7409):111-115; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein by reference in their entireties.

Examples of oligonucleotides for promoting DMPK gene editing include US Patent Application Publication 20170088819A1, published on Mar. 3, 2017, entitled “Genetic Correction Of Myotonic Dystrophy Type I”; and International Patent Application Publication WO18002812A1, published on Apr. 1, 2018, entitled “Materials And Methods For Treatment Of Myotonic Dystrophy Type 1 (DM1) And Other Related Disorders,” the contents of each of which are incorporated herein by reference in their entireties.

In some embodiments, the oligonucleotide may have region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46; and Machuca-Tzili L. et al. “Clinical and molecular aspects of the myotonic dystrophies: a review.” Muscle Nerve. 2005 July; 32(1):1-18; the contents of each of which are incorporated herein by reference in their entireties.

In some embodiments, an oligonucleotide provided herein is an antisense oligonucleotide targeting DMPK. In some embodiments, the oligonucleotide targeting is any one of the antisense oligonucleotides (e.g., a Gapmer) targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference. In some embodiments, the DMPK targeting oligonucleotide targets a region of the DMPK gene sequence as set forth in Genbank accession No. NM_001081560.2 or as set forth in Genbank accession No. NG_009784.1.

In some embodiments, the DMPK targeting oligonucleotide comprises a nucleotide sequence comprising a region complementary to a target region that is at least 10 continuous nucleotides (e.g., at least 10, at least 12, at least 14, at least 16, or more continuous nucleotides) in Genbank accession No. NM_001081560.2.

In some embodiments, the DMPK targeting oligonucleotide comprise a gapmer motif. “Gapmer” means a chimeric antisense compound in which an internal region having a plurality of nucleotides that support RNase H cleavage is positioned between external regions having one or more nucleotides, wherein the nucleotides comprising the internal region are chemically distinct from the nucleotide or nucleotides comprising the external regions. The internal region can be referred to as a “gap segment” and the external regions can be referred to as “wing segments.” In some embodiments, the DMPK targeting oligonucleotide comprises one or more modified nucleotides, and/or one or more modified internucleotide linkages. In some embodiments, the internucleotide linkage is a phosphorothioate linkage. In some embodiments, the oligonucleotide comprises a full phosphorothioate backbone. In some embodiments, the oligonucleotide is a DNA gapmer with cET ends (e.g., 3-10-3; cET-DNA-cET). In some embodiments, the DMPK targeting oligonucleotide comprises one or more 6′-(S)—CH3 bicyclic nucleotides, one or more β-D-2′-deoxyribonucleotides, and/or one or more 5-methylcytosine nucleotides.

DUX4/FSHD

In some embodiments, examples of oligonucleotides useful for targeting DUX4, e.g., for the treatment of FSHD, are provided in U.S. Pat. No. 9,988,628, published on Feb. 2, 2017, entitled “AGENTS USEFUL IN TREATING FACIOSCAPULOHUMERAL MUSCULAR DYSTROPHY”; U.S. Pat. No. 9,469,851, published Oct. 30, 2014, entitled “RECOMBINANT VIRUS PRODUCTS AND METHODS FOR INHIBITING EXPRESSION OF DUX4”; US Patent Application Publication 20120225034, published on Sep. 6, 2012, entitled “AGENTS USEFUL IN TREATING FACIOSCAPULOHUMERAL MUSCULAR DYSTROPHY”; PCT Patent Application Publication Number WO 2013/120038, published on Aug. 15, 2013, entitled “MORPHOLINO TARGETING DUX4 FOR TREATING FSHD”; Chen et al., “Morpholino-mediated Knockdown of DUX4 Toward Facioscapulohumeral Muscular Dystrophy Therapeutics,” Molecular Therapy, 2016, 24:8, 1405-1411; and Ansseau et al., “Antisense Oligonucleotides Used to Target the DUX4 mRNA as Therapeutic Approaches in Facioscapulohumeral Muscular Dystrophy (FSHD),” Genes, 2017, 8, 93; the contents of each of which are incorporated herein in their entireties. In some embodiments, the oligonucleotide is an antisense oligonucleotide, a morpholino, a siRNA, a shRNA, or another nucleotide which hybridizes with the target DUX4 gene or mRNA.

In some embodiments, e.g., for the treatment of FSHD, oligonucleotides may have a region of complementarity to a hypomethylated, contracted D4Z4 repeat, as in Daxinger, et al., “Genetic and Epigenetic Contributors to FSHD,” published in Curr Opin Genet Dev in 2015, Lim J-W, et al., DICER/AGO-dependent epigenetic silencing of D4Z4 repeats enhanced by exogenous siRNA suggests mechanisms and therapies for FSHD Hum Mol Genet. 2015 Sep. 1; 24(17): 4817-4828, the contents of each of which are incorporated in their entireties.

DNM2/CNM

In some embodiments, examples of oligonucleotides useful for targeting DNM2, e.g., for the treatment of CNM, are provided in US Patent Application Publication Number 20180142008, published on May 24, 2018, entitled “DYNAMIN 2 INHIBITOR FOR THE TREATMENT OF DUCHENNE'S MUSCULAR DYSTROPHY”, and in PCT Application Publication Number WO 2018/100010A1, published on Jun. 7, 2018, entitled “ALLELE-SPECIFIC SILENCING THERAPY FOR DYNAMIN 2-RELATED DISEASES”. For example, in some embodiments, the oligonucleotide is a RNAi, an antisense nucleic acid, a siRNA, or a ribozyme that interferes specifically with DNM2 expression. Other examples of oligonucleotides useful for targeting DNM2 are provided in Tasfaout, et al., “Single Intramuscular Injection of AAV-shRNA Reduces DNM2 and Prevents Myotubular Myopathy in Mice,” published in Mol. Ther, on Apr. 4, 2018, and in Tasfaout, et al., “Antisense oligonucleotide-mediated Dnm2 knockdown prevents and reverts myotubular myopathy in mice,” Nature Communications volume 8, Article number: 15661 (2017). In some embodiments, the oligonucleotide is a shRNA or a morpholino that efficiently targets DNM2 mRNA. In some embodiments, the oligonucleotide encodes wild-type DNM2 which is resistant to miR-133 activity, as in Todaka, et al. “Overexpression of NF90-NF45 Represses Myogenic MicroRNA Biogenesis, Resulting in Development of Skeletal Muscle Atrophy and Centronuclear Muscle Fibers,” published in Mol. Cell Biol, in July 2015 Further examples of oligonucleotides useful for targeting DNM2 are provided in Gibbs, et al., “Two Dynamin-2 Genes are Required for Normal Zebrafish Development” published in PLoS One in 2013, the contents of each of which are incorporated herein in their entirety.

In some embodiments, e.g., for the treatment of CNM, the oligonucleotide may have a region of complementarity to a mutant in DNM2 associated with CNM, as in Böhm et al, “Mutation Spectrum in the Large GTPase Dynamin 2, and Genotype-Phenotype Correlation in Autosomal Dominant Centronuclear Myopathy,” as published in Hum. Mutat, in 2012, the contents of which are incorporated herein in its entirety.

Pompe Disease

In some embodiments, e.g., for the treatment of Pompe disease, an oligonucleotide mediates exon 2 inclusion in a GAA disease allele as in van der Wal, et al., “GAA Deficiency in Pompe Disease is Alleviated by Exon Inclusion in iPSC-Derived Skeletal Muscle Cells,” Mol Ther Nucleic Acids. 2017 Jun. 16; 7: 101-115, the contents of which are incorporated herein by reference. Accordingly, in some embodiments, the oligonucleotide may have a region of complementarity to a GAA disease allele.

In some embodiments, e.g., for the treatment of Pompe disease, an oligonucleotide, such as an RNAi or antisense oligonucleotide, is utilized to suppress expression of wild-type GYS1 in muscle cells, as reported, for example, in Clayton, et al., “Antisense Oligonucleotide-mediated Suppression of Muscle Glycogen Synthase 1 Synthesis as an Approach for Substrate Reduction Therapy of Pompe Disease,” published in Mol Ther Nucleic Acids in 2017, or US Patent Application Publication Number 2017182189, published on Jun. 29, 2017, entitled “INHIBITING OR DOWNREGULATING GLYCOGEN SYNTHASE BY CREATING PREMATURE STOP CODONS USING ANTISENSE OLIGONUCLEOTIDES”, the contents of which are incorporated herein by reference. Accordingly, in some embodiments, oligonucleotides may have an antisense strand having a region of complementarity to a sequence a human GYS1 sequence, corresponding to RefSeq number NM_002103.4 and/or a mouse GYS1 sequence, corresponding to RefSeq number NM_030678.3.

ACVR1/FOP

In some embodiments, examples of oligonucleotides useful for targeting ACVR1, e.g., for the treatment of FOP, are provided in US Patent Application 2009/0253132, published Oct. 8, 2009, “Mutated ACVR1 for diagnosis and treatment of fibrodysplasia ossificans progressiva (FOP)”; WO 2015/152183, published Oct. 8, 2015, “Prophylactic agent and therapeutic agent for fibrodysplasia ossificans progressive”; Lowery, J. W. et al, “Allele-specific RNA Interference in FOP-Silencing the FOP gene”, GENE THERAPY, vol. 19, 2012, pages 701-702; Takahashi, M. et al. “Disease-causing allele-specific silencing against the ALK2 mutants, R206H and G356D, in fibrodysplasia ossificans progressiva” Gene Therapy (2012) 19, 781-785; Shi, S. et al. “Antisense-Oligonucleotide Mediated Exon Skipping in Activin-Receptor-Like Kinase 2: Inhibiting the Receptor That Is Overactive in Fibrodysplasia Ossificans Progressiva” Plos One, July 2013, Vol 8:7, e69096; US Patent Application 2017/0159056, published Jun. 8, 2017, “Antisense oligonucleotides and methods of use thereof”; U.S. Pat. No. 8,859,752, issued Oct. 4, 2014, “SIRNA-based therapy of Fibrodyplasia Ossificans Progressiva (FOP)”; WO 2004/094636, published Nov. 4, 2004, “Effective sirna knock-down constructs”, the contents of each of which are incorporated herein in their entireties.

FXN/Friedreich's Ataxia

In some embodiments, examples of oligonucleotides useful for targeting FXN and/or otherwise compensating for frataxin deficiency, e.g., for the treatment of Freidreich Ataxia, are provided in Li, L. et al “Activating frataxin expression by repeat-targeted nucleic acids” Nat. Comm. 2016, 7:10606; WO 2016/094374, published Jun. 16, 2016, “Compositions and methods for treatment of friedreich's ataxia.”; WO 2015/020993, published Feb. 12, 2015, “RNAi COMPOSITIONS AND METHODS FOR TREATMENT OF FRIEDREICH'S ATAXIA”; WO 2017/186815, published Nov. 2, 2017, “Antisense oligonucleotides for enhanced expression of frataxin”: WO 2008/018795, published Feb. 14, 2008, “Methods and means for treating dna repeat instability associated genetic disorders”; US Patent Application 2018/0028557, published Feb. 1, 2018, “Hybrid oligonucleotides and uses thereof”; WO 2015/023975, published Feb. 19, 2015, “Compositions and methods for modulating RNA”; WO 2015/023939, published Feb. 19, 2015, “Compositions and methods for modulating expression of frataxin”; US Patent Application 2017/0281643, published Oct. 5, 2017, “Compounds and methods for modulating frataxin expression”; Li L. et al., “Activating frataxin expression by repeat-targeted nucleic acids” Nature Communications, Published 4 Feb. 2016; and Li L. et al. “Activation of Frataxin Protein Expression by Antisense Oligonucleotides Targeting the Mutant Expanded Repeat” Nucleic Acid Ther. 2018 February; 28(1):23-33., the contents of each of which are incorporated herein in their entireties.

In some embodiments, an oligonucleotide payload is configured (e.g., as a gapmer or RNAi oligonucleotide) for inhibiting expression of a natural antisense transcript that inhibits FXN expression, e.g., as disclosed in U.S. Pat. No. 9,593,330, filed Jun. 9, 2011. “Treatment of frataxin (FXN) related diseases by inhibition of natural antisense transcript to FXN”, the contents of which are incorporated herein by reference in its entirety.

Examples of oligonucleotides for promoting FXN gene editing include WO 2016/094845, published Jun. 16, 2016, “Compositions and methods for editing nucleic acids in cells utilizing oligonucleotides”; WO 2015/089354, published Jun. 18, 2015, “Compositions and methods of use of CRISPR-Cas systems in nucleotide repeat disorders”; WO 2015/139139, published Sep. 24, 2015, “CRISPR-based methods and products for increasing frataxin levels and uses thereof”; and WO 2018/002783, published Jan. 4, 2018, “Materials and methods for treatment of Friedreich ataxia and other related disorders”, the contents of each of which are incorporated herein in their entireties.

Examples of oligonucleotides for promoting FXN gene expression through targeting of non-FXN genes, e.g. epigenetic regulators of FXN, include WO 2015/023938, published Feb. 19, 2015, “Epigenetic regulators of frataxin”, the contents of which are incorporated herein in its entirety.

In some embodiments, oligonucleotides may have a region of complementarity to a sequence set forth as: an FXN gene from humans (Gene ID 2395; NC_000009.12) and/or an FXN gene from mice (Gene ID 14297; NC_000085.6). In some embodiments, the oligonucleotide may have region of complementarity to a mutant form of FXN, for example as reported in e.g., Montermini, L. et al. “The Friedreich ataxia GAA triplet repeat: premutation and normal alleles.” Hum. Molec. Genet., 1997, 6: 1261-1266; Filla, A. et al. “The relationship between trinucleotide (GAA) repeat length and clinical features in Friedreich ataxia.” Am. J. Hum. Genet. 1996, 59: 554-560; Pandolfo, M. Friedreich ataxia: the clinical picture. J. Neurol. 2009, 256, 3-8; the contents of each of which are incorporated herein by reference in their entireties.

DMD/Dystrophinopathies

Examples of oligonucleotides useful for targeting DMD are provided in U.S. Patent Application Publication US20100130591A1, published on May 27, 2010, entitled “MULTIPLE EXON SKIPPING COMPOSITIONS FOR DMD”; U.S. Pat. No. 8,361,979, issued Jan. 29, 2013, entitled “MEANS AND METHOD FOR INDUCING EXON-SKIPPING”; U.S. Patent Application Publication 20120059042, published Mar. 8, 2012, entitled “METHOD FOR EFFICIENT EXON (44) SKIPPING IN DUCHENNE MUSCULAR DYSTROPHY AND ASSOCIATED MEANS; U.S. Patent Application Publication 20140329881, published Nov. 6, 2014, entitled “EXON SKIPPING COMPOSITIONS FOR TREATING MUSCULAR DYSTROPHY”; U.S. Pat. No. 8,232,384, issued Jul. 31, 2012, entitled “ANTISENSE OLIGONUCLEOTIDES FOR INDUCING EXON SKIPPING AND METHODS OF USE THEREOF”; U.S. Patent Application Publication 20120022134A1, published Jan. 26, 2012, entitled “METHODS AND MEANS FOR EFFICIENT SKIPPING OF EXON 45 IN DUCHENNE MUSCULAR DYSTROPHY PRE-MRNA; U.S. Patent Application Publication 20120077860, published Mar. 29, 2012, entitled “ADENO-ASSOCIATED VIRAL VECTOR FOR EXON SKIPPING IN A GENE ENCODING A DISPENSABLE DOMAN PROTEIN”; U.S. Pat. No. 8,324,371, issued Dec. 4, 2012, entitled “OLIGOMERS”; U.S. Pat. No. 9,078,911, issued Jul. 14, 2015, entitled “ANTISENSE OLIGONUCLEOTIDES”; U.S. Pat. No. 9,079,934, issued Jul. 14, 2015, entitled “ANTISENSE NUCLEIC ACIDS”; U.S. Pat. No. 9,034,838, issued May 19, 2015, entitled “MIR-31 IN DUCHENNE MUSCULAR DYSTROPHY THERAPY”; and International Patent Publication WO2017062862A3, published Apr. 13, 2017, entitled “OLIGONUCLEOTIDE COMPOSITIONS AND METHODS THEREOF”; the contents of each of which are incorporated herein in their entireties.

Examples of oligonucleotides for promoting DMD gene editing include International Patent Publication WO2018053632A1, published Mar. 29, 2018, entitled “METHODS OF MODIFYING THE DYSTROPHIN GENE AND RESTORING DYSTROPHIN EXPRESSION AND USES THEREOF”; International Patent Publication WO2017049407A1, published Mar. 30, 2017, entitled “MODIFICATION OF THE DYSTROPHIN GENE AND USES THEREOF”; International Patent Publication WO2016161380A1, published Oct. 6, 2016, entitled “CRISPR/CAS-RELATED METHODS AND COMPOSITIONS FOR TREATING DUCHENNE MUSCULAR DYSTROPHY AND BECKER MUSCULAR DYSTROPHY”; International Patent Publication WO2017095967, published Jun. 8, 2017, entitled “THERAPEUTIC TARGETS FOR THE CORRECTION OF THE HUMAN DYSTROPHIN GENE BY GENE EDITING AND METHODS OF USE”; International Patent Publication WO2017072590A1, published May 4, 2017, entitled “MATERIALS AND METHODS FOR TREATMENT OF DUCHENNE MUSCULAR DYSTROPHY”; International Patent Publication WO2018098480A1, published May 31, 2018, entitled “PREVENTION OF MUSCULAR DYSTROPHY BY CRISPR/CPF1-MEDIATED GENE EDITING”; US Patent Application Publication US20170266320A1, published Sep. 21, 2017, entitled “RNA-Guided Systems for In Vivo Gene Editing”; International Patent Publication WO2016025469A1, published Feb. 18, 2016, entitled “PREVENTION OF MUSCULAR DYSTROPHY BY CRISPR/CAS9-MEDIATED GENE EDITING”; U.S. Patent Application Publication 2016/0201089, published Jul. 14, 2016, entitled “RNA-GUIDED GENE EDITING AND GENE REGULATION”; and U.S. Patent Application Publication 2013/0145487, published Jun. 6, 2013, entitled “MEGANUCLEASE VARIANTS CLEAVING A DNA TARGET SEQUENCE FROM THE DYSTROPHN GENE AND USES THEREOF”, the contents of each of which are incorporated herein in their entireties. In some embodiments, an oligonucleotide may have a region of complementarity to DMD gene sequences of multiple species, e.g., selected from human, mouse and non-human species.

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

MYH7/Hypertrophic Cardiomyopathy

Examples of oligonucleotides useful as payloads, e.g., for targeting MYH7 are provided in US Patent Application Publication 20180094262, published on Apr. 5, 2018, entitled Inhibitors of MYH7B and Uses Thereof; US Patent Application Publication 20160348103, published on Dec. 1, 2016, entitled Oligonucleotides and Methods for Treatment of Cardiomyopathy Using RNA Interference; US Patent Application Publication 20160237430, published on Aug. 18, 2016, entitled “Allele-specific RNA Silencing for the Treatment of Hypertrophic Cardiomyopathy”; US Patent Application Publication 20160032286, published on Feb. 4, 2016, entitled “Inhibitors of MYH7B and Uses Thereof”; US Patent Application Publication 20140187603, published on Jul. 3, 2014, entitled “MicroRNA Inhibitors Comprising Locked Nucleotides”; US Patent Application Publication 20140179764, published on Jun. 26, 2014, entitled “Dual Targeting of miR-208 and miR-499 in the Treatment of Cardiac Disorders”; US Patent Application Publication 20120114744, published on May 10, 2012, entitled “Compositions and Methods to Treat Muscular and Cardiovascular Disorders”; the contents of each of which are incorporated herein in their entireties.

In some embodiments, the oligonucleotide may target lncRNA or mRNA, e.g., for degradation. In some embodiments, the oligonucleotide may target, e.g., for degradation, a nucleic acid encoding a protein involved in a mismatch repair pathway, e.g., MSH2, MutLalpha, MutSbeta, MutLalpha. Non-limiting examples of proteins involved in mismatch repair pathways, for which mRNAs encoding such proteins may be targeted by oligonucleotides described herein, are described in Iyer, R. R. et al., “DNA triplet repeat expansion and mismatch repair” Annu Rev Biochem. 2015; 84:199-226; and Schmidt M. H. and Pearson C. E., “Disease-associated repeat instability and mismatch repair” DNA Repair (Amst). 2016 February; 38:117-26.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In a some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an target nucleic acid. In some embodiments a complementary nucleotide sequence need not be 100% complementary to that of its target to be specifically hybridizable or specific for a target nucleic acid.

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of any one of the oligonucleotides provided herein. In some embodiments, such target sequence is 100% complementary to the oligonucleotide described herein.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein may independently and optionally be uracil bases (U's), and/or any one or more of the U's in the oligonucleotides provided herein may independently and optionally be T's.

b. Oligonucleotide Modifications:

The oligonucleotides described herein may be modified, e.g., comprise a modified sugar moiety, a modified internucleoside linkage, a modified nucleotide and/or combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

c. Modified Nucleotides

In some embodiments, an oligonucleotide include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA).

In some embodiments, an oligonucleotide can include at least one 2′-O-methyl-modified nucleotide, and in some embodiments, all of the nucleotides include a 2′-O-methyl modification. In some embodiments, an oligonucleotide comprises modified nucleotides in which the ribose ring comprises a bridge moiety connecting two atoms in the ring, e.g., connecting the 2′-O atom to the 4′-C atom. In some embodiments, the oligonucleotides are “locked,” e.g., comprise modified nucleotides in which the ribose ring is “locked” by a methylene bridge connecting the 2′-O atom and the 4′-C atom. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety.

Other modifications that may be used in the oligonucleotides disclosed herein include ethylene-bridged nucleic acids (ENAs). ENAs include, but are not limited to, 2′-0,4′-C-ethylene-bridged nucleic acids. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties.

In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. In some embodiments, the oligonucleotide comprises a modified nucleotide disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA.

In some embodiments, the oligonucleotide may have at least one modified nucleotide that results in an increase in Tm of the oligonucleotide in a range of 1° C., 2° C., 3° C., 4° C., or 5° C. compared with an oligonucleotide that does not have the at least one modified nucleotide. The oligonucleotide may have a plurality of modified nucleotides that result in a total increase in Tm of the oligonucleotide in a range of 2° C., 3° C., 4° C., 5° C., 6° C., 7° C. 8° C., 9° C., 10° C., 15° C., 20° C., 25° C., 30° C., 35° C., 40° C., 45° C. or more compared with an oligonucleotide that does not have the modified nucleotide.

The oligonucleotide may comprise alternating nucleotides of different kinds. For example, an oligonucleotide may comprise alternating deoxyribonucleotides or ribonucleotides and 2′-fluoro-deoxyribonucleotides. An oligonucleotide may comprise alternating deoxyribonucleotides or ribonucleotides and 2′-O-methyl nucleotides. An oligonucleotide may comprise alternating 2′-fluoro nucleotides and 2′-O-methyl nucleotides. An oligonucleotide may comprise alternating bridged nucleotides and 2′-fluoro or 2′-O-methyl nucleotides.

d. Internucleotide Linkages/Backbones

In some embodiments, oligonucleotide may contain a phosphorothioate or other modified internucleotide linkage. In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides. For example, in some embodiments, oligonucleotides comprise modified internucleotide linkages at the first, second, and/or third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., one to six modified nucleotides. Examples of modified nucleotides include, but are not limited to, 2′ MOE or 2′OMe or Locked Nucleic Acid bases (LNA). The flanking sequences X and Z may be of one to twenty nucleotides, one to eight nucleotides or one to five nucleotides in length, in some embodiments. The flanking sequences X and Z may be of similar length or of dissimilar lengths. The gap-segment Y may be a nucleotide sequence of five to twenty nucleotides, size to twelve nucleotides or six to ten nucleotides in length, in some embodiments.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

i. Mixmers

In some embodiments, an oligonucleotide described herein may be a mixmer or comprise a mixmer sequence pattern. In general, mixmers are oligonucleotides that comprise both naturally and non-naturally occurring nucleotides or comprise two different types of non-naturally occurring nucleotides typically in an alternating pattern. Mixmers generally have higher binding affinity than unmodified oligonucleotides and may be used to specifically hind a target molecule, e.g., to block a binding site on the target molecule. Generally, mixmers do not recruit an RNAse to the target molecule and thus do not promote cleavage of the target molecule. Such oligonucleotides that are incapable of recruiting RNAse H have been described, for example, see WO2007/112754 or WO2007/112753.

In some embodiments, the mixmer comprises or consists of a repeating pattern of nucleotide analogues and naturally occurring nucleotides, or one type of nucleotide analogue and a second type of nucleotide analogue. However, a mixmer need not comprise a repeating pattern and may instead comprise any arrangement of modified nucleotides and naturally occurring nucleotides or any arrangement of one type of modified nucleotide and a second type of modified nucleotide. The repeating pattern, may, for instance be every second or every third nucleotide is a modified nucleotide, such as LNA, and the remaining nucleotides are naturally occurring nucleotides, such as DNA, or are a 2′ substituted nucleotide analogue such as 2′MOE or 2′ fluoro analogues, or any other modified nucleotide described herein. It is recognized that the repeating pattern of modified nucleotide, such as LNA units, may be combined with modified nucleotide at fixed positions—e.g, at the 5′ or 3′ termini.

In some embodiments, a mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive modified nucleotides, such as at least two consecutive LNAs. In some embodiments, the mixmer comprises at least a region consisting of at least three consecutive modified nucleotide units, such as at least three consecutive LNAs.

In some embodiments, the mixmer does not comprise a region of more than 7, more than 6, more than 5, more than 4, more than 3, or more than 2 consecutive nucleotide analogues, such as LNAs. In some embodiments. LNA units may be replaced with other nucleotide analogues, such as those referred to herein.

Mixmers may be designed to comprise a mixture of affinity enhancing modified nucleotides, such as in non-limiting example LNA nucleotides and 2′-O-methyl nucleotides. In some embodiments, a mixmer comprises modified internucleoside linkages (e.g., phosphorothioate internucleoside linkages or other linkages) between at least two, at least three, at least four, at least five or more nucleotides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleotides. For example, in some embodiments, a mixmer may comprise morpholino nucleotides mixed (e.g., in an alternating manner) with one or more other nucleotides (e.g., DNA. RNA nucleotides) or modified nucleotides (e.g., LNA, 2′-O-Methyl nucleotides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

j. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence. siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

l. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotide molecules. For example, Benseler et al. (J. Am. Chem. Soc. (1993) 115:8483-8484) disclosed hammerhead-like molecules in which two of the base pairs of stem I1, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotide, ethylene glycol-related linkers. Thomson et al. (Nucleic Acids Res. (1993) 21:5600-5603) replaced loop II with linear, non-nucleotide linkers of 13, 17, and 19 atoms in length.

Ribozyme oligonucleotides can be prepared using well known methods (sec, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

n. Guide Nucleic Adds

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase 111.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Splice Altering Oligonucleotides

In some embodiments, an oligonucleotide (e.g., an antisense oligonucleotide including a morpholino) of the present disclosure target splicing. In some embodiments, the oligonucleotide targets splicing by inducing exon skipping and restoring the reading frame within a gene. As a non-limiting example, the oligonucleotide may induce skipping of an exon encoding a frameshift mutation and/or an exon that encodes a premature stop codon. In some embodiments, an oligonucleotide may induce exon skipping by blocking spliceosome recognition of a splice site. In some embodiments, exon skipping results in a truncated but functional protein compared to the reference protein (e.g., truncated but functional DMD protein as described below). In some embodiments, the oligonucleotide promotes inclusion of a particular exon (e.g., exon 7 of the SMN2 gene described below). In some embodiments, an oligonucleotide may induce inclusion of an exon by targeting a splice site inhibitory sequence. RNA splicing has been implicated in muscle diseases, including Duchenne muscular dystrophy (DMD) and spinal muscular atrophy (SMA).

Alterations (e.g., deletions, point mutations, and duplications) in the gene encoding dystrophin (DMD) cause DMD. These alterations can lead to frameshift mutations and/or nonsense mutations. In some embodiments, an oligonucleotide of the present disclosure promotes skipping of one or more DMD exons (e.g., exon 8, exon 43, exon 44, exon 45, exon 50, exon 51, exon 52, exon 53, and/or exon 55) and results in a functional truncated protein. See, e.g., U.S. Pat. No. 8,486,907 published on Jul. 16, 2013 and U.S. 20140275212 published on Sep. 18, 2014.

In SMA, there is loss of functional SMN1. Although the SMN2 gene is a paralog to SMN1, alternative splicing of the SMN2 gene predominantly leads to skipping of exon 7 and subsequent production of a truncated SMN protein that cannot compensate for SMN1 loss. In some embodiments, an oligonucleotide of the present disclosure promotes inclusion of SMN2 exon 7. In some embodiments, an oligonucleotide is an antisense oligonucleotide that targets SMN2 splice site inhibitory sequences (see, e.g., U.S. Pat. No. 7,838,657, which was published on Nov. 23, 2010).

p. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

C. Linkers

Complexes described herein generally comprise a linker that connects any one of the muscle targeting agents (e.g., anti-TfR antibodies) described herein to a molecular payload. A linker comprises at least one covalent bond. In some embodiments, a linker may be a single bond, e.g., a disulfide bond or disulfide bridge, that connects a muscle targeting agent (e.g., anti-TfR antibody) to a molecular payload. However, in some embodiments, a linker may connect any one of the muscle targeting agents (e.g., anti-TfR antibodies) described herein to a molecular through multiple covalent bonds. In some embodiments, a linker may be a cleavable linker. However, in some embodiments, a linker may be a non-cleavable linker. A linker is generally stable in vitro and in vivo, and may be stable in certain cellular environments. Additionally, generally a linker does not negatively impact the functional properties of either the anti-TfR antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ADC Linker Chemistry” Pharm Res. 2015, 32:11, 3526-3540; McCombs, J. R, and Owen, S. C. “Antibody Drug Conjugates: Design and Selection of Linker, Payload and Conjugation Chemistry” AAPS J. 2015, 17:2, 339-351).

A precursor to a linker typically will contain two different reactive species that allow for attachment to both the anti-TfR antibody and a molecular payload. In some embodiments, the two different reactive species may be a nucleophile and/or (e.g., and) an electrophile. In some embodiments, a linker is connected to a muscle targeting agent (e.g., anti-TfR antibody) via conjugation to a lysine residue or a cysteine residue of the anti-TfR antibody. In some embodiments, a linker is connected to a cysteine residue of a muscle targeting agent (e.g., anti-TfR antibody) via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle targeting agent (e.g., anti-TfR antibody) or thiol functionalized molecular payload via a 3-arylpropionitrile functional group. In some embodiments, a linker is connected to a lysine residue of a muscle targeting agent (e.g., anti-TfR antibody). In some embodiments, a linker is connected to a muscle targeting agent (e.g., anti-TfR antibody) and/or (e.g., and) a molecular payload via an amide bond, a carbamate bond, a hydrazide, a trizaole, a thioether, or a disulfide bond.

i. Cleavable Linkers

A cleavable linker may be a protease-sensitive linker, a pH-sensitive linker, or a glutathione-sensitive linker. These linkers are generally cleavable only intracellularly and are preferably stable in extracellular environments, e.g. extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g, a cysteine residue.

In some embodiments, the linker is a Val-cit linker (e.g., as described in U.S. Pat. No. 6,214,345, incorporated hircine by reference). In some embodiments, before conjugation, the val-cit linker has a structure of:

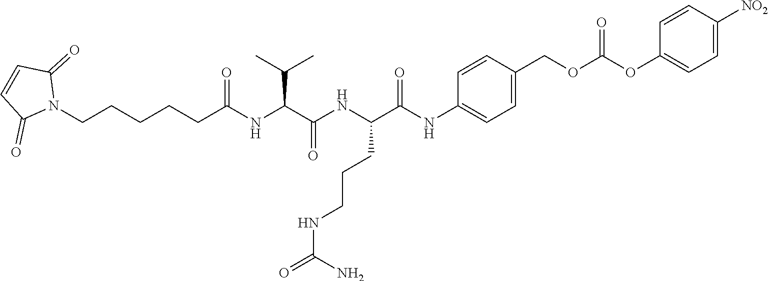 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, the val-cit linker has a structure of:

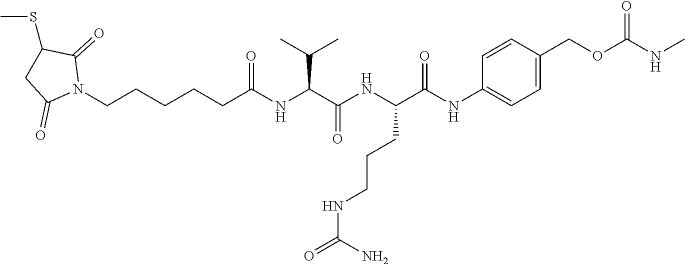

In some embodiments, the Val-cit linker is attached to a reactive chemical moiety (e.g., SPAAC for click chemistry conjugation). In some embodiments, before click chemistry conjugation, the val-cit linker attached to a reactive chemical moiety (e.g., SPAAC for click chemistry conjugation) has the structure of:

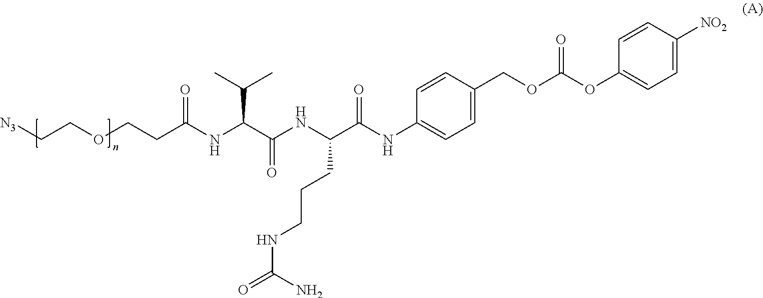

wherein n is any number from 0-15. In some embodiments, n is 3.

In some embodiments, the val-cit linker attached to a reactive chemical moiety (e.g., SPAAC for click chemistry conjugation) is conjugated (e.g., via a different chemical moiety) to a molecular payload (e.g., an oligonucleotide). In some embodiments, the val-cit linker attached to a reactive chemical moiety (e.g., SPAAC for click chemistry conjugation) and is conjugated to a molecular payload (e.g., an oligonucleotide) has the structure of (before click chemistry conjugation):

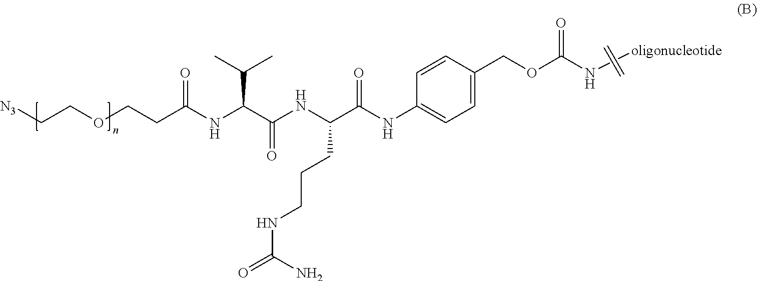

wherein n is any number from 0-15. In some embodiments, n is 3.

In some embodiments, after conjugation to a molecular payload (e.g., an oligonucleotide), the val-cit linker comprises a structure of:

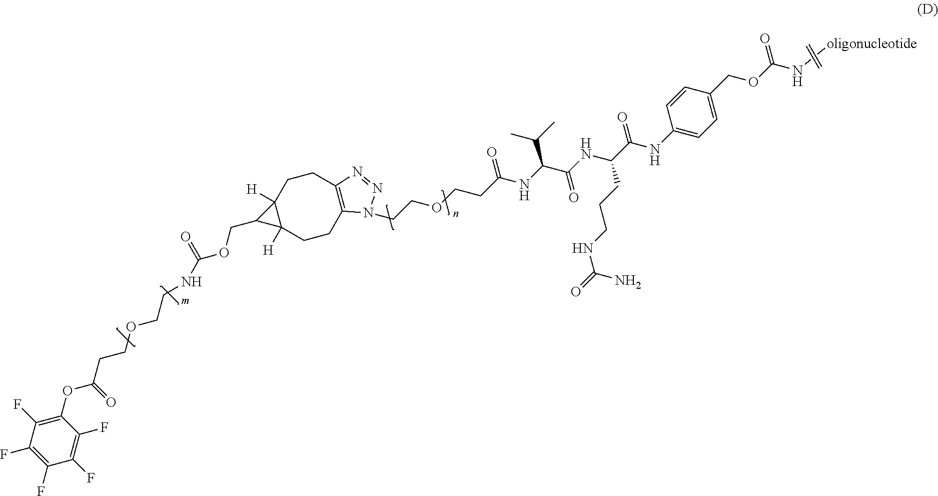

wherein n is any number from 0-15, and wherein m is any number from 0-15. In some embodiments, n is 3 and m is 4.

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or (e.g., and) an alkoxy-amine linker. In some embodiments, sortase-mediated ligation will be utilized to covalently link a muscle targeting agent (e.g., anti-TfR antibody) comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species O, an optionally substituted sulfur species, or a poly(alkylene oxide), e.g. polyethylene oxide or polypropylene oxide.

iii. Linker Conjugation

In some embodiments, a linker is connected to a muscle targeting agent (e.g., anti-TfR antibody) and/or (e.g., and) molecular payload via a phosphate, thioether, ether, carbon-carbon, carbamate, or amide bond. In some embodiments, a linker is connected to an oligonucleotide through a phosphate or phosphorothioate group, e.g, a terminal phosphate of an oligonucleotide backbone. In some embodiments, a linker is connected to a muscle targeting agent (e.g., anti-TfR antibody), through a lysine or cysteine residue present on the muscle targeting agent (e.g., anti-TfR antibody).

In some embodiments, a linker is connected to a muscle targeting agent (e.g., anti-TfR antibody) and/or (e.g., and) molecular payload by a cycloaddition reaction between an azide and an alkyne to form a triazole, wherein the azide and the alkyne may be located on the muscle targeting agent (e.g., anti-TfR antibody), molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctane is as described in International Patent Application Publication WO2011136645, published on Nov. 3, 2011, entitled, “Fused Cyclooctyne Compounds And Their Use In Metal-free Click Reactions”. Both the exo- and endo-forms of BCN may be used for link conjugation and the BCN in Formulae (C), (D), (E), (F), (G), and (H) provided herein can be either endo- or exo-BCN.

In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide and the alkyne may be located on the muscle targeting agent (e.g., anti-TfR antibody), molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker further comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is connected to a muscle targeting agent (e.g., anti-TfR antibody) and/or (e.g., and) molecular payload by the Diels-Alder reaction between a dienophile and a diene/hetero-diene, wherein the dienophile and the diene/hetero-diene may be located on the muscle targeting agent (e.g., anti-TfR antibody), molecular payload, or the linker. In some embodiments a linker is connected to a muscle targeting agent (e.g., anti-TfR antibody) and/or (e.g., and) molecular payload by other pericyclic reactions, e.g. ene reaction. In some embodiments, a linker is connected to a muscle targeting agent (e.g., anti-TfR antibody) and/or (e.g., and) molecular payload by an amide, thioamide, or sulfonamide bond reaction. In some embodiments, a linker is connected to a muscle targeting agent (e.g., anti-TfR antibody) and/or (e.g., and) molecular payload by a condensation reaction to form an oxime, hydrazone, or semicarbazide group existing between the linker and the muscle targeting agent (e.g., anti-TfR antibody) and/or (e.g., and) molecular payload.

In some embodiments, a linker is connected to a muscle targeting agent (e.g., anti-TfR antibody) and/or (e.g., and) molecular payload by a conjugate addition reactions between a nucleophile, e.g, an amine or a hydroxyl group, and an electrophile, e.g, a carboxylic acid, carbonate, or an aldehyde. In some embodiments, a nucleophile may exist on a linker and an electrophile may exist on a muscle targeting agent (e.g., anti-TfR antibody) or molecular payload prior to a reaction between a linker and a muscle targeting agent (e.g., anti-TfR antibody) or molecular payload. In some embodiments, an electrophile may exist on a linker and a nucleophile may exist on a muscle targeting agent (e.g., anti-TfR antibody) or molecular payload prior to a reaction between a linker and a muscle targeting agent (e.g., anti-TfR antibody) or molecular payload. In some embodiments, an electrophile may be an azide, pentafluorophenyl, para-nitrophenol ester, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or (e.g., and) an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

In some embodiments, the val-cit linker attached to a reactive chemical moiety (e.g., SPAAC for click chemistry conjugation) is conjugated to the anti-TfR antibody by a structure of:

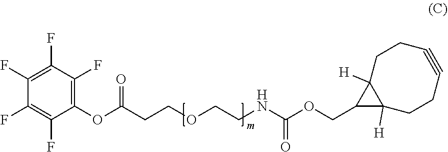

wherein m is any number from 0-15. In some embodiments, m is 4.

In some embodiments, the val-cit linker attached to a reactive chemical moiety (e.g., SPAAC for click chemistry conjugation) is conjugated to an anti-TfR antibody having a structure of:

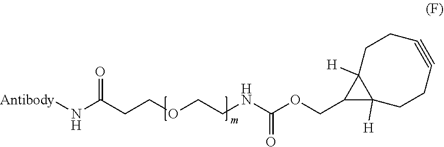

wherein m is any number from 0-15. In some embodiments, m is 4.

In some embodiments, the val-cit linker attached to a reactive chemical moiety (e.g., SPAAC for click chemistry conjugation) and is conjugated to an anti-TfR antibody has a structure of:

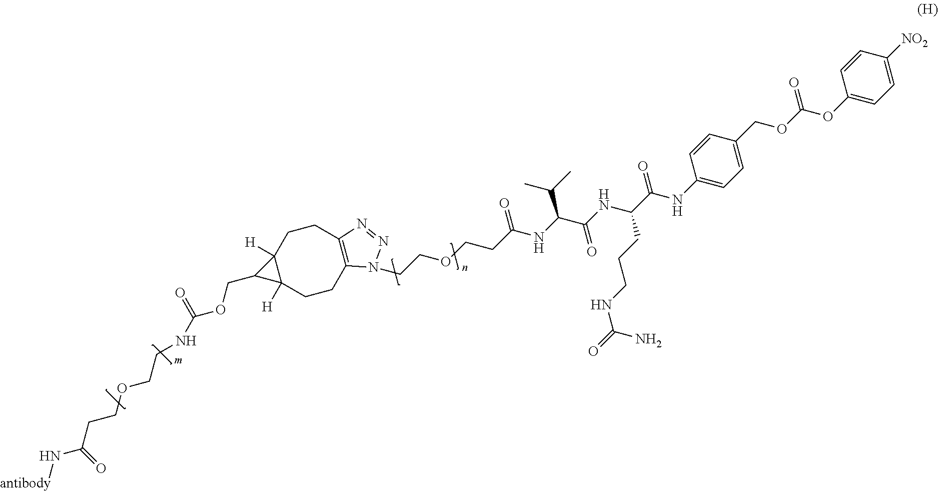

wherein n is any number from 0-15, wherein m is any number from 0-15. In some embodiments, n is 3 and/or (e.g., and) m is 4. In some embodiments, an oligonucleotide is covalently linked to a compound comprising a structure of formula (H), thereby forming a complex comprising a structure of formula (E). It should be understood that the amide shown adjacent the anti-TfR1 antibody in Formula (H) results from a reaction with an amine of the anti-TfR1 antibody, such as a lysine epsilon amine.

In some embodiments, the val-cit linker used to covalently link an anti-TfR antibody and a molecular payload (e.g., an oligonucleotide) comprises a structure of:

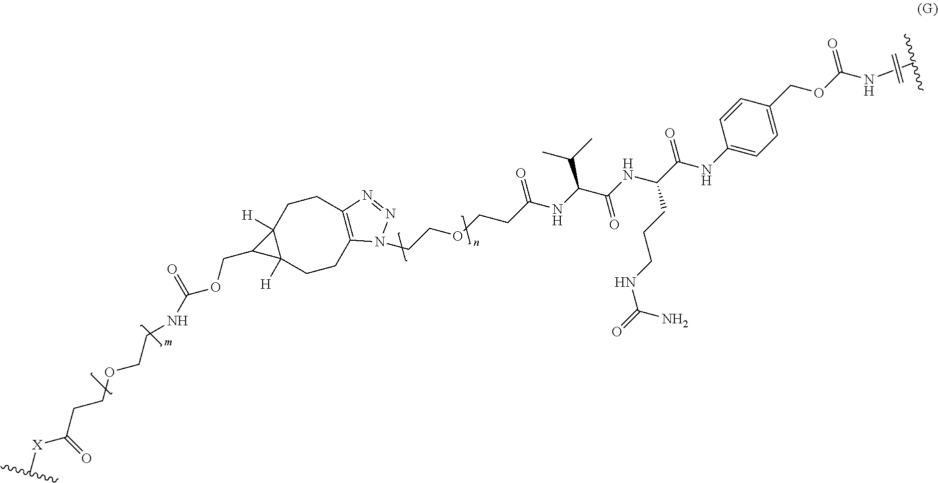

wherein n is any number from 0-15, wherein m is any number from 0-15. In some embodiments, n is 3 and/or (e.g., and) m is 4. In some embodiments, n is 3 and/or (e.g., and) m is 4. In some embodiments, X is NH (e.g., NH from an amine group of a lysine), S (e.g., S from a thiol group of a cysteine), or O (e.g., O from a hydroxyl group of a serine, threonine, or tyrosine) of the antibody. In some embodiments, the method of processing complexes described comprises steps of producing the complexes.

In some embodiments, the method of producing the complexes comprises:

(i) obtaining an oligonucleotide comprising a structure of:

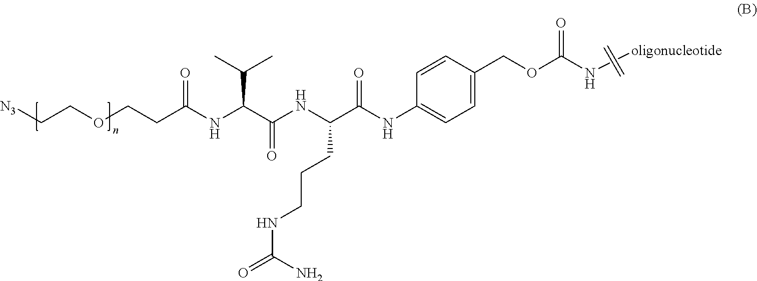

wherein n is 0-15 (e.g., 3);

(ii) obtaining an antibody comprises a structure of:

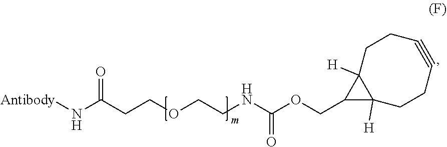

wherein m is 0-15 (e.g., 4); and

(iii) reacting the oligonucleotide in step (i) and the antibody obtained in step (ii) to obtain the complex.

In some embodiments, the method comprises:

(i) obtaining an oligonucleotide comprising a structure of:

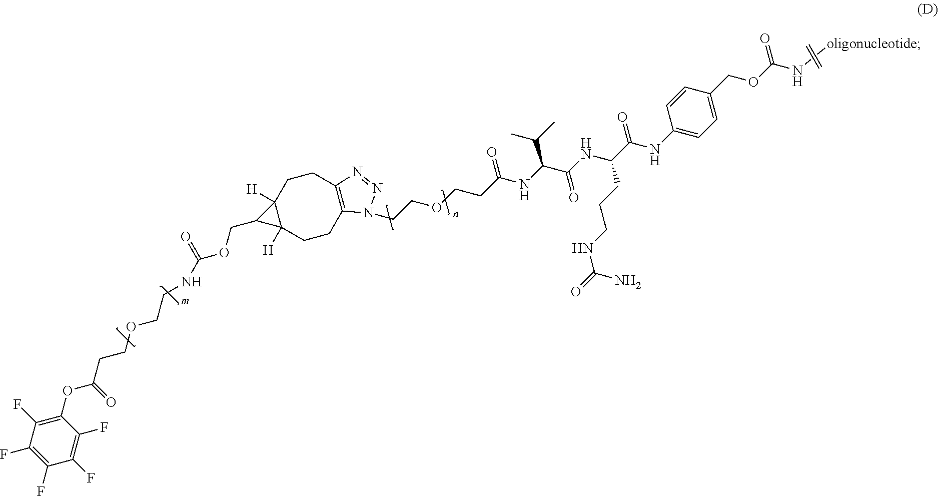

wherein n is 0-15 (e.g., 3) and wherein m is 0-15 (e.g., 4);

(ii) obtaining an antibody; and

(iii) reacting the oligonucleotide in step (i) and the antibody obtained in step (ii) to obtain the complex.

In some embodiments, the complexes produced using the methods described herein comprises a structure of:

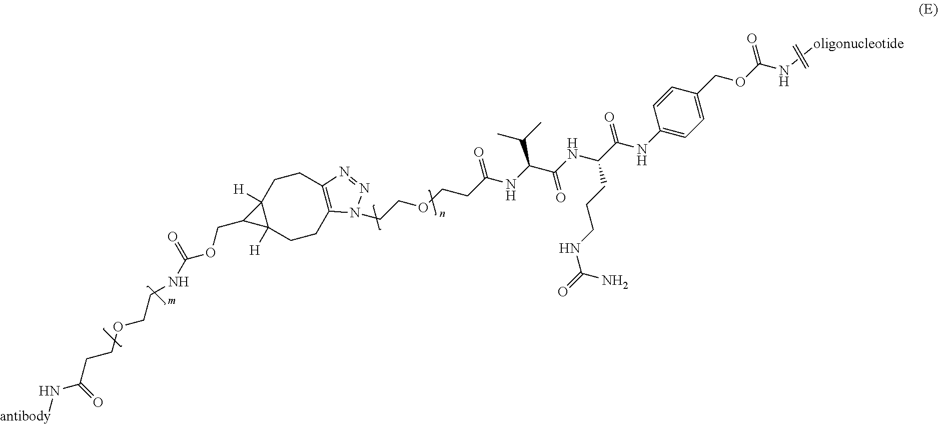

wherein n is 0-15 (e.g., 3) and wherein m is 0-15 (e.g., 4), and wherein the antibody is covalently linked via a lysine.

In some embodiments, the complex described herein has a structure of:

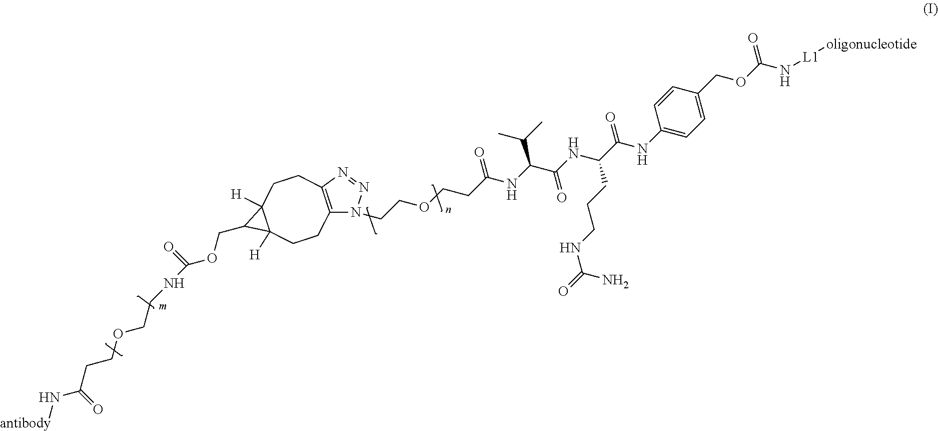

wherein n is any number from 0-10, wherein m is any number from 0-10. In some embodiments, n is 3 and m is 4.

In structure formula (I), L1 is, in some embodiments, a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted hetermarylene, —O—, —N(R^{A})—, —S—, —C(═O)—, —C(═O)O—, —C(═O)NR^{A}—, —NR^{A}C(═O)—, —NR^{C}(═O)R^{A}—, —C(═O)R^{A}—, —NR^{C}(═O)O—, —NR^{C}(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

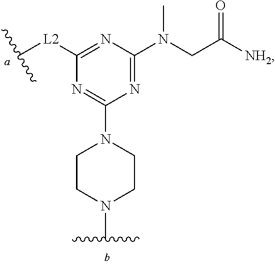

wherein L2 is

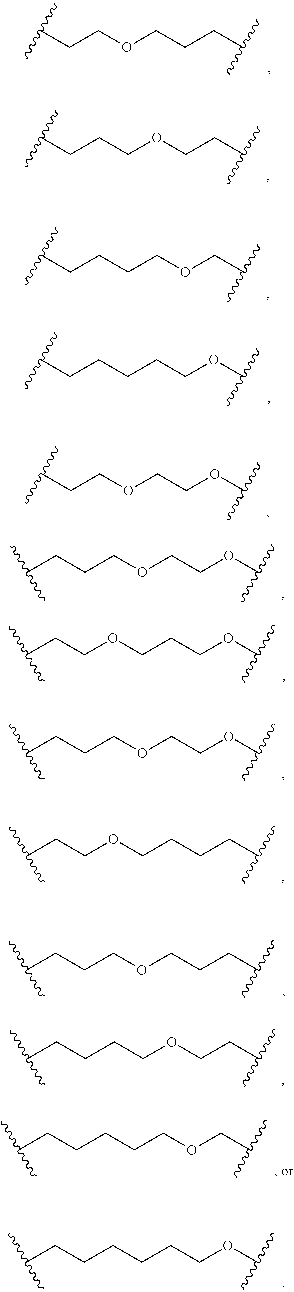

wherein a labels the site directly linked to the carbamate moiety of Formula (I); and b labels the site covalently linked (directly or via additional chemical moieties) to the oligonucleotide.

In some embodiments, L1 is:

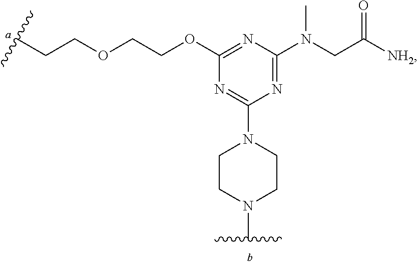 [SMILES: *CCOCCOc1nc(N(C)CC(N)=O)nc(N2CCN(*)CC2)n1]

wherein a labels the site directly linked to the carbamate moiety of Formula (I); and b labels the site covalently linked (directly or via additional chemical moieties) to the oligonucleotide.

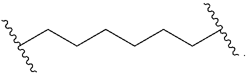 [SMILES: *CCCCCC*]

In some embodiments, Li is

In some embodiments, Li is linked to a 5′ phosphate of the oligonucleotide.

In some embodiments, Li is optional (e.g., need not be present).

It should be understood that the amide shown adjacent the anti-TfR1 antibody in Formula (E), Formula (F), and Formula (I) results from a reaction with an amine of the anti-TfR1 antibody, such as a lysine epsilon amine.

D. Examples of Antibody-Molecular Payload Complexes

Further provided herein are non-limiting examples of complexes comprising any one the muscle targeting agents (e.g., anti-TfR antibodies) described herein covalently linked to any of the molecular payloads (e.g., an oligonucleotide) described herein. In some embodiments, the anti-TfR antibody (e.g., any one of the muscle targeting agents (e.g., anti-TfR antibodies) provided in Table 2) is covalently linked to a molecular payload (e.g., an oligonucleotide) via a linker. Any of the linkers described herein may be used. In some embodiments, if the molecular payload is an oligonucleotide, the linker is covalently linked to the 5′ end, the 3′ end, or internally of the oligonucleotide. In some embodiments, the linker is covalently linked to the anti-TfR antibody via a thiol-reactive linkage (e.g., via a cysteine in the anti-TfR antibody). In some embodiments, the linker (e.g., a Val-cit linker) is covalently linked to the antibody (e.g., an anti-TfR antibody described herein) via a n amine group (e.g., via a lysine in the antibody). In some embodiments, the molecular payload is an oligonucleotide (e.g., an oligonucleotide targeting an atrophy gene listed in Table 1).

An example of a structure of a complex comprising an anti-TfR antibody covalently linked to a molecular payload via a Val-cit linker is provided below:

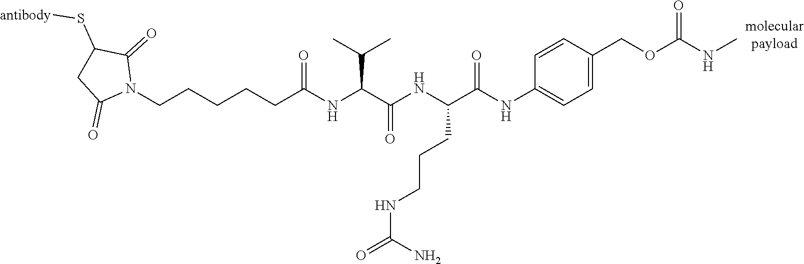

wherein the linker is covalently linked to the antibody via a thiol-reactive linkage (e.g., via a cysteine in the antibody). In some embodiments, the molecular payload is an oligonucleotide (e.g., an oligonucleotide targeting an atrophy gene listed in Table 1).

Another example of a structure of a complex comprising an anti-TfR antibody covalently linked to a molecular payload via a Val-cit linker is provided below:

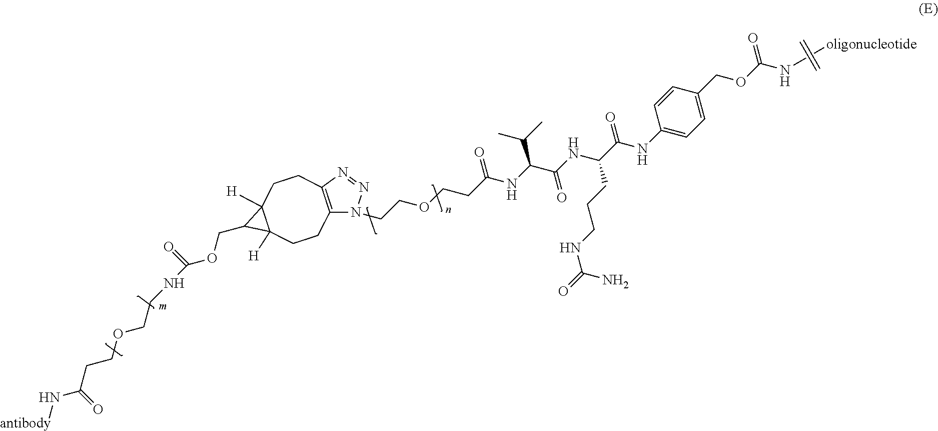

wherein n is a number between 0-15, wherein m is a number between 0-15, wherein the linker is covalently linked to the antibody via an amine group (e.g., on a lysine residue), and/or (e.g., and) wherein the linker is covalently linked to the oligonucleotide (e.g., at the 5′ end, 3′ end, or internally). In some embodiments, the linker is covalently linked to the antibody via a lysine, the linker is covalently linked to the oligonucleotide at the 5′ end, n is 3, and m is 4. In some embodiments, the molecular payload is an oligonucleotide (e.g., an oligonucleotide targeting an atrophy gene listed in Table 1).

It should be appreciated that antibodies can be covalently linked to oligonucleotides with different stoichiometries, a property that may be referred to as a drug to antibody ratios (DAR) with the “drug” being the oligonucleotide. In some embodiments, one oligonucleotide is covalently linked to an antibody (DAR=1). In some embodiments, two oligonucleotides are covalently linked to an antibody (DAR=2). In some embodiments, three oligonucleotides are covalently linked to an antibody (DAR=3). In some embodiments, four oligonucleotides are covalently linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating oligonucleotides to different sites on an antibody and/or by conjugating multimers to one or more sites on antibody. For example, a DAR of 2 may be achieved by conjugating a single oligonucleotide to two different sites on an antibody or by conjugating a dimer oligonucleotide to a single site of an antibody.

In some embodiments, the complex described herein comprises a transferrin receptor antibody (e.g., an antibody or any variant thereof as described herein) covalently linked to an oligonucleotide. In some embodiments, the complex described herein comprises a transferrin receptor antibody (e.g., an antibody or any variant thereof as described herein) covalently linked to an oligonucleotide via a linker (e.g., a Val-cit linker). In some embodiments, the linker (e.g., a Val-cit linker) is covalently linked to the 5′ end, the 3′ end, or internally of the oligonucleotide. In some embodiments, the linker (e.g., a Val-cit linker) is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3.

In some embodiments, the complex described herein comprises a transferrin receptor antibody covalently linked to an oligonucleotide, wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to an oligonucleotide, wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to an oligonucleotide, wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to an oligonucleotide, wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to an oligonucleotide via a linker (e.g., a Val-cit linker), wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3.

In some embodiments, the complex described herein comprises a transferrin receptor antibody covalently linked to an oligonucleotide via a linker (e.g., a Val-cit linker), wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to an oligonucleotide via a linker (e.g., a Val-cit linker), wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to an oligonucleotide via a linker (e.g., a Val-cit linker), wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to an oligonucleotide via a linker (e.g., a Val-cit linker), wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to an oligonucleotide via a Val-cit linker, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3, and wherein the complex comprises the structure of:

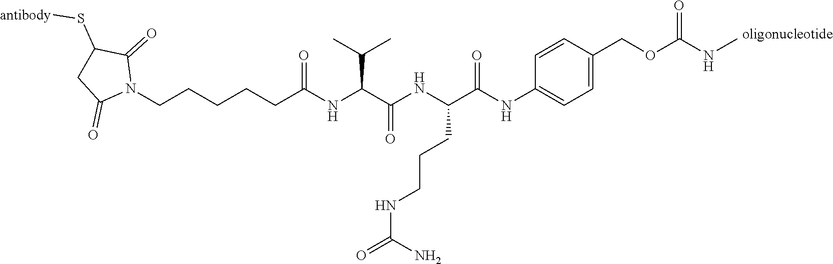

wherein the linker Val-cit linker is covalently linked to the 5′ end, the 3′ end, or internally of the oligonucleotide, and wherein the Val-cit linker is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 33 and a VL having the amino acid sequence of SEQ ID NO: 34, and wherein the complex comprises the structure of:

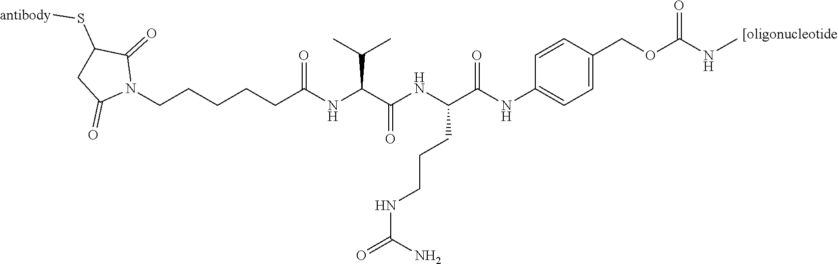

wherein the linker Val-cit linker is covalently linked to the 5′ end, the 3′ end, or internally of the oligonucleotide, and wherein the Val-cit linker is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 35 and a VL having the amino acid sequence of SEQ ID NO: 36, and wherein the complex comprises the structure of:

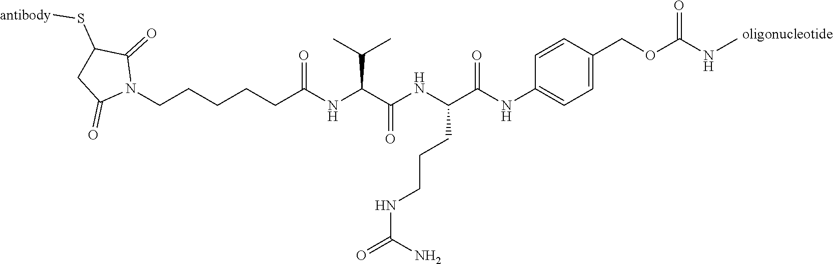

wherein the linker Val-cit linker is covalently linked to the 5′ end, the 3′ end, or internally of the oligonucleotide, and wherein the Val-cit linker is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 39 and a light chain having the amino acid sequence of SEQ ID NO: 40, and wherein the complex comprises the structure of:

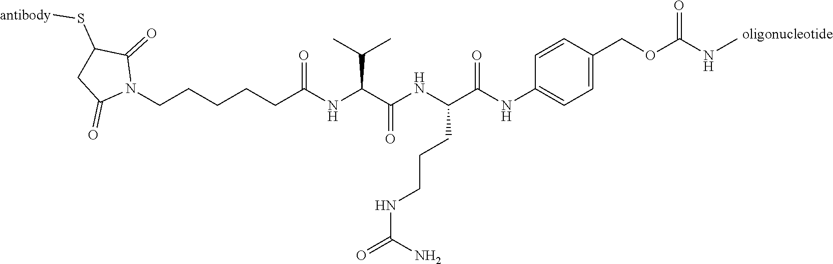

wherein the linker Val-cit linker is covalently linked to the 5′ end, the 3′ end, or internally of an oligonucleotide, and wherein the Val-cit linker is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 41 and a light chain having the amino acid sequence of SEQ ID NO: 42, and wherein the complex comprises the structure of:

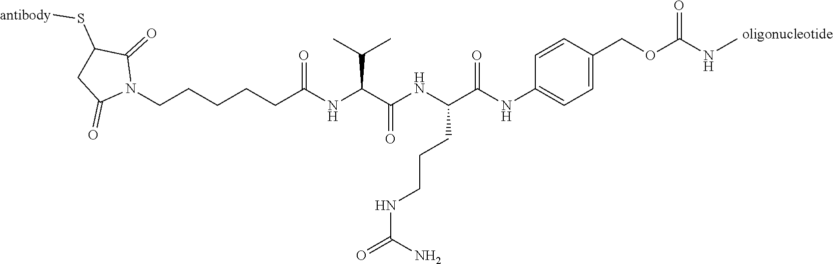

wherein the linker Val-cit linker is covalently linked to the 5′end, the 3′end, or internally of an oligonucleotide, and wherein the Val-cit linker is covalently linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked via a lysine to the 5′ end of an oligonucleotide, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3, and wherein the complex comprises the structure of:

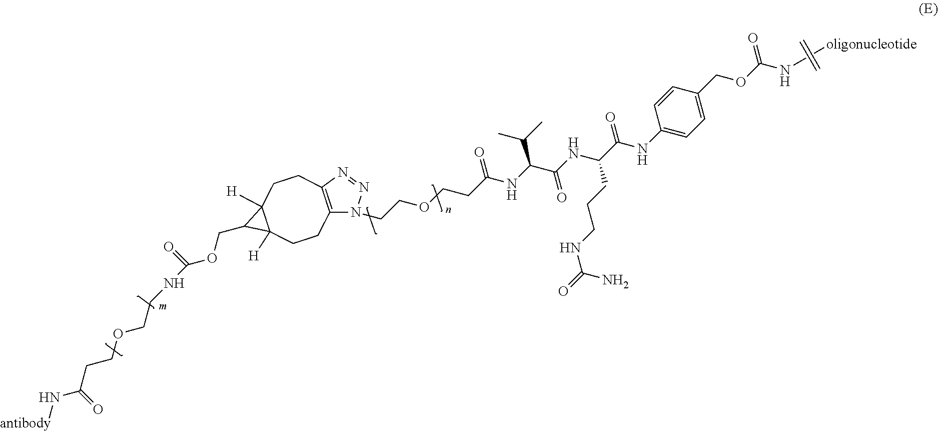

wherein n is 3 and m is 4. In some embodiments, the oligonucleotide is an oligonucleotide targeting an atrophy gene listed in Table 1.

In some embodiments, the complex described herein comprises a transferrin receptor antibody covalently linked via a lysine to the 5′ end of an oligonucleotide, wherein the transferrin receptor antibody comprises a VH having the amino acid sequence of SEQ ID NO: 33 and a VL having the amino acid sequence of SEQ ID NO: 34, and wherein the complex comprises the structure of:

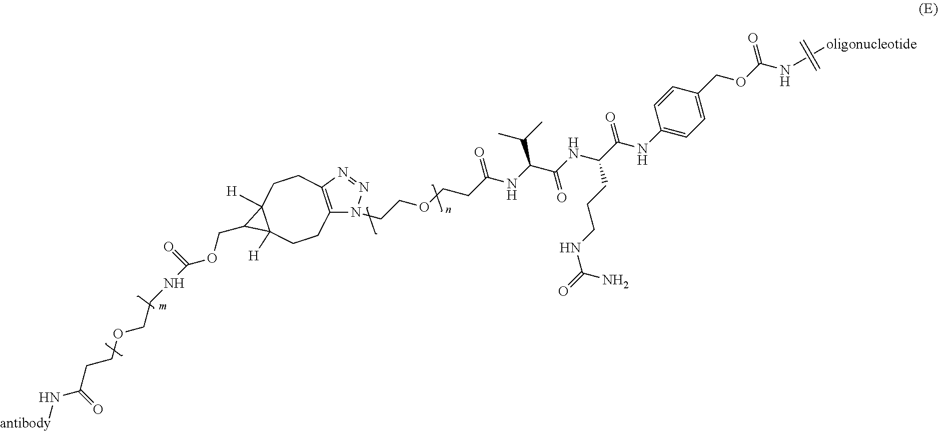

wherein n is 3 and m is 4. In some embodiments, the oligonucleotide is an oligonucleotide targeting an atrophy gene listed in Table 1. In some embodiments, the complex described herein comprises a transferrin receptor antibody covalently linked via a lysine to the 5′ end of an oligonucleotide, wherein the transferrin receptor antibody comprises a VH having the amino acid sequence of SEQ ID NO: 35 and a VL having the amino acid sequence of SEQ ID NO: 36, and wherein the complex comprises the structure of:

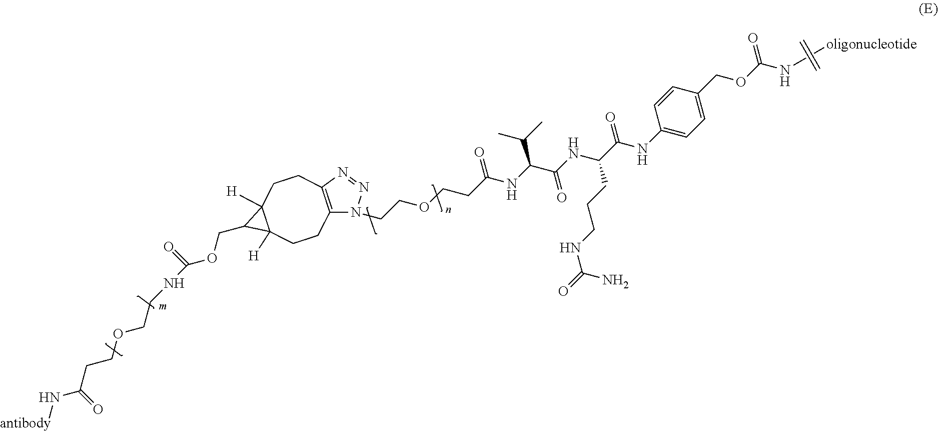

wherein n is 3 and m is 4. In some embodiments, the oligonucleotide is an oligonucleotide targeting an atrophy gene listed in Table 1.

In some embodiments, the complex described herein comprises a transferrin receptor antibody covalently linked via a lysine to the 5′ end of an oligonucleotide, wherein the transferrin receptor antibody comprises a heavy chain having the amino acid sequence of SEQ ID NO: 39 and a light chain having the amino acid sequence of SEQ ID NO: 40, and wherein the complex comprises the structure of:

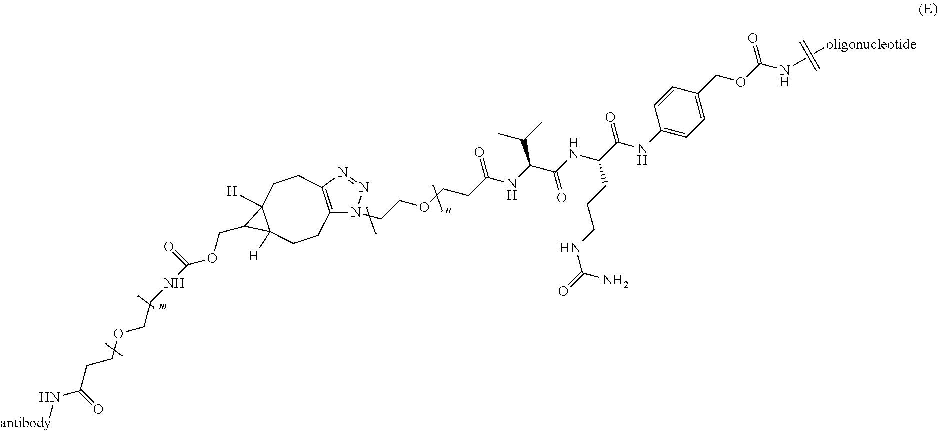

wherein n is 3 and m is 4. In some embodiments, the oligonucleotide is an oligonucleotide targeting an atrophy gene listed in Table 1.

In some embodiments, the complex described herein comprises a transferrin receptor antibody covalently linked via a lysine to the 5′ end of an oligonucleotide, wherein the transferrin receptor antibody comprises a heavy chain having the amino acid sequence of SEQ ID NO: 41 and a light chain having the amino acid sequence of SEQ ID NO: 42, and wherein the complex comprises the structure of:

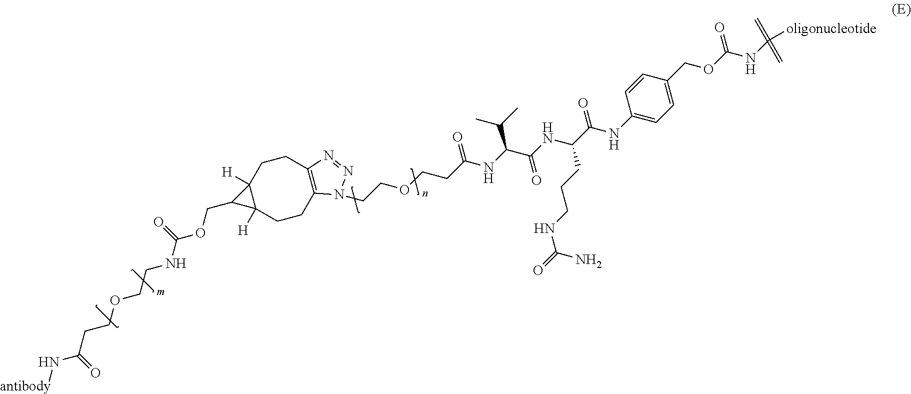

wherein n is 3 and m is 4. In some embodiments, the oligonucleotide is an oligonucleotide targeting an atrophy gene listed in Table 1.

It should be understood that the amide shown adjacent the anti-TfR1 antibody in the examples of complexes having Formula (E) results from a reaction with an amine of the anti-TfR1 antibody, such as a lysine epsilon amine.

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous. In some embodiments, the route of administration is extramuscular parenteral administration.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the a complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently to a molecular payload as described herein are effective in treating a muscle disease (e.g., a ram muscle disease). In some embodiments, complexes are effective in treating a muscle disease provided in Table 1. In some embodiments, a muscle disease is associated with a disease allele, for example, a disease allele for a particular muscle disease may comprise a genetic alteration in a corresponding gene listed in Table 1.

In some embodiments, a subject may be a human subject, a non-human primate subject, a rodent subject, or any suitable mammalian subject. In some embodiments, a subject may have a muscle disease provided in Table 1.

An aspect of the disclosure includes a method involving administering to a subject an effective amount of a complex as described herein. In some embodiments, an effective amount of a pharmaceutical composition that comprises a complex comprising a muscle-targeting agent covalently to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently to a molecular payload is administered at an effective concentration that confers therapeutic effect on a subject. Effective amounts vary, as recognized by those skilled in the art, depending on the severity of the disease, unique characteristics of the subject being treated, e.g, age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g. safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment

The efficacy of treatment may be assessed using any suitable methods. In some embodiments, the efficacy of treatment may be assessed by evaluation of observation of symptoms associated with a muscle disease.

In some embodiments, a pharmaceutical composition that comprises a complex comprising a muscle-targeting agent covalently to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a pharmaceutical composition may comprise more than one complex comprising a muscle-targeting agent covalently to a molecular payload. In some embodiments, a pharmaceutical composition may further comprise any other suitable therapeutic agent for treatment of a subject, e.g, a human subject having a muscle disease (e.g., a muscle disease provided in Table 1). In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Synthesis of a Complex Comprising an Antibody Linked to an Oligonucleotide (Conjugation Method 1—Pre-Reaction Conjugation)

A muscle-targeting complex was generated comprising an antisense oligonucleotide that targets DMPK covalently linked, via a cathepsin cleavable linker, to an anti-transferrin receptor hIgG1-kappa Fab antibody (anti-TfR Fab).

The anti-TfR Fab was purified from CHO cell culture supernatant, using 1 mL CaptureSelect™ CH1-XL columns (Thermo Fisher, Loughborough, UK). The columns were washed using 1×DPBS and protein was subsequently eluted using 50 mM sodium acetate pH 4.0. The protein was subsequently buffer exchanged into 20 mM sodium citrate, 150 mM NaCl. pH 6.0. The Fab was then further purified by preparative SEC using a HiLoad™ 16/60 Superdex™ 75 pg column (GE Healthcare, Little Chalfont, UK) with 20 mM sodium citrate, 150 mM NaCl, pH 6.0 as the mobile phase. Peak fractions containing monomeric protein were pooled, concentrated and filter sterilized before quantification according to A280 nm using an extinction coefficient (Ec(0.1%)) based on the predicted amino acid sequence. Purified Fab was then analyzed by reducing and non-reducing sodium dodecyl sulfate-polyacrylamide gel electrophoresis (SDS-PAGE), analytical size-exclusion chromatography-high performance liquid chromatography (SEC-HPLC) and Biacore single-cycle kinetics (SCK). Prior to continuing to the conjugation reaction, the anti-TfR Fab was buffer exchanged into 50 mM HEPES pH 7.5.

A linker/payload compound comprising the oligonucleotide (e.g., charged oligonucleotide) and an azide-Valine-Citrulline linker was generated. The oligonucleotide (Na+ adduct) was dissolved at 200 mg/mL in RNAse free water. This solution was diluted to 10 mg/mL with dry dimethylformamide (DMF). A 25-fold molar excess of tributylamine was then added to the solution. The linker molecule (azide-PEG3-Val-Cit-PAB-PNP, dissolved at 20 mg/mL in DMF) was added at a 2-fold molar excess to the oligonucleotide solution for 120 min at room temperature (˜25° C.). Reaction completion was measured using ninhydrin (Kaiser test) prior to quenching the reaction using an alcohol precipitation. The alcohol precipitation was accomplished by addition of 0.1 v/v 3 M NaCl solution, followed by addition of 3 volumes of −80° C. isopropanol. The solution was then thoroughly mixed and subsequently allowed to precipitate at −20° C. for 1 hour. The precipitated solution was centrifuged (at 4300× g; 8° C.) for 30 mins and the solvent was decanted. The pellet was washed with −80° C. 80% ethanol in RNase free water (one volume equivalent to starting reaction) and centrifuged (at 4300× g; 8° C.) for 20 min. Ethanol was then decanted and the pellet (containing the compound comprising the oligonucleotide and an azide-Valine-Citrulline linker) was dried for 10 min at 37° C. The linker/payload compound comprising the oligonucleotide and azide-Valine-Citrulline linker was resuspended in 20% acetonitrile in nuclease free water to a concentration of 20 mg/mL.

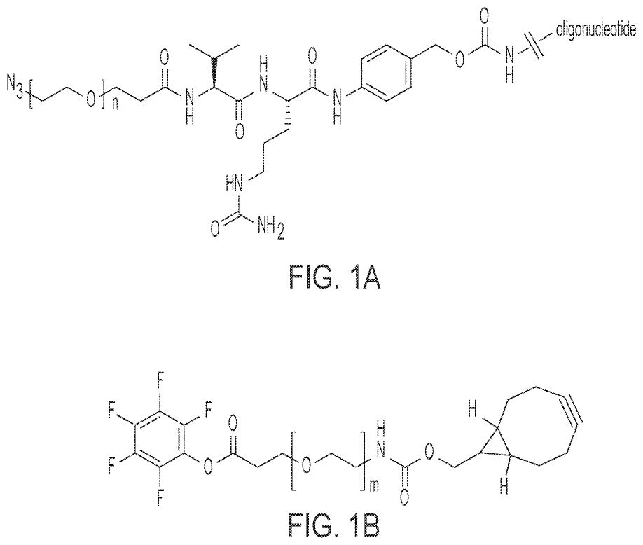

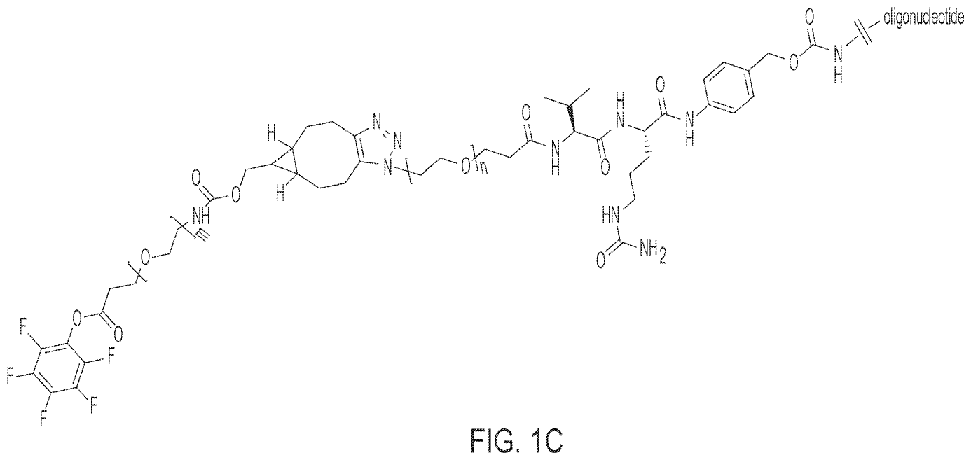

A pre-reaction was subsequently conducted with the following conditions: 5.87 μM, oligonucleotide-PAB-VC-PEG3-azide (structure shown in FIG. 1A) and 5.34 mM endo-BCN-PEG4-PFP ester (1.1:1.0 mol:mol equivalents; structure shown in FIG. 1B) were combined in 60:40 v/v % DMA to 25 mM 2-(N-morpholino)ethanesulfonic acid (MES) pH 5.5 buffer at room temperature.

Completion of the pre-reaction to generate oligonucleotide-PAB-VC-PEG3-triazole-PEG4-PFP ester (structure shown in FIG. 1C) was monitored by RP-C18 UPLC with repeat injections every 30 minutes (at 5 minute, 35 minute, and 65 minute time points), by observing the disappearance of the endo-BCN-PEG4-PFP ester starting material at 220 nm. The reaction was determined to be complete when less than 5% of the endo-BCN-PEG4-PFP ester was remaining with respect to the 5 minute time point. The crude pre-reaction mixture was carried forward to the conjugation reaction immediately without any purification. The total pre-reaction time was 90 minutes, defined as the time between addition of the endo-BCN-PEG4-PFP ester to the pre-reaction and addition of this reaction mixture to the Fab conjugation reaction.

Conjugation of the oligonucleotide (e.g., charged oligonucleotide) to the anti-TfR Fab involves the formation of an amide bond between solvent accessible lysine residues of the Fab and the activated PFP ester of the oligonucleotide-PAB-VC-PEG3-triazole-PEG4-PFP ester that was generated in the pre-reaction step.

The conjugation reaction was conducted with the following solution reactant parameters: anti-TfR Fab (45 mg, 6 mg/mL, 125 μM) and oligonucleotide-PAB-VC-PEG3-triazole-PEG4-PFP ester at a final concentration of 750 μM (1.0:6.0 mol:mol equivalents of anti-TfR Fab to oligonucleotide-PAB-VC-PEG3-triazole-PEG4-PFP ester). The oligonucleotide-PAB-VC-PEG3-triazole-PEG4-PFP ester concentration assumes 100% conversion of the BCN in the pre-reaction into oligonucleotide-PAB-VC-PEG3-triazole-PEG4-PFP ester. The final reaction mixture consisted of 15:85 v/v % DMA to 25 mM HEPES pH 7.5 buffer. The reaction was set up in a 20 mL glass scintillation vial by adding the appropriate amounts of the reactants and stock solutions. The reaction proceeded for 20 hr at room temperature (˜25° C.). The start of the reaction was defined as the addition time of the pre-reaction mixture containing the oligonucleotide-PAB-VC-PEG3-triazole-PEG4-PFP ester to the anti-TfR Fab solution. The stop time was defined as the beginning of the pH adjustment prior to subsequent purification. The total duration of the conjugation reaction was 20 hours.

After reacting for 20 hours the crude conjugate mixture was tested by SDS-PAGE and analyzed by densitometry to determine the drug to antibody ratio (DAR) and % unconjugated Fab.

### Example 2: Purification of Complexes Comprising Muscle-Targeting Antibodies Linked to Oligonucleotides

The crude reaction product from Example 1 was successfully purified using chromatography. The mixture of anti-TfR Fab-oligonucleotide conjugate, unconjugated anti-TfR Fab and unconjugated oligonucleotide was diluted 1:3 in nuclease free water and the pH of the mixture was adjusted to 5.7 with 500 mM MES buffer. This solution was then loaded onto a ceramic hydroxyapatite (HA) column (HiLoad-26 mm×40 cm column, CHT™ 40 μm resin, from Biorad; Catalog #732-4324) at a biomolecule concentration of 8 mg/mL of resin [load flow rate: linear 113 cm/hr with 26 mm ID-10.0 mL/min volumetric flow rate, residence time-21.4 minutes]. The HA column was washed with 5 CV of a wash solution (5 mM Na₂HPO₄, 25 mM NaCl pH 7.0) to remove unlinked oligonucleotide. Following removal of the unlinked oligonucleotide, the complex comprising anti-TfR Fab covalently linked to oligonucleotide was eluted from the HA column in formulation buffer (100 mM Na₂HPO₄, 100 mM NaCl, pH 7.6).

Isolated and purified anti-TfR Fab-oligonucleotide conjugate was analyzed by SDS-PAGE and analytical SEC to demonstrate complete removal of unlinked oligonucleotide, as well as by ELISA for human TfR1/cyno TfR1 binding and for endotoxin levels. SEC chromatograms showed that the elution fraction from the HA resin provided substantially purified complex comprising anti-TfR Fab covalently linked to oligonucleotide. Further, the HA flow-through (e.g., the wash fraction) comprised unlinked oligonucleotide. These results demonstrate that the complex can be purified from the unlinked oligonucleotide using hydroxyapatite resin, a surprising purification result that was otherwise unattainable.

Several alternative strategies for the purification of the crude mixture of a complex comprising anti-TfR Fab covalently linked to oligonucleotide, unlinked oligonucleotide, and unlinked anti-TfR Fab from Example 1 were examined, including cation exchange (CEX) and anion exchange (AEX) resins. It was found that none of the alternative strategies was as effective as the approach described here (using ceramic hydroxyapatite resin).

### Example 3: Effect of the Ratio of BCN to Fab on Fab-Oligonucleotide Conjugation Reactions

To investigate the effect of the ratio of BCN from the pre-reaction mixture to anti-TfR, a set of reactions were conducted according to the general protocol described in Example 1.

The pre-reaction between oligonucleotide-PAB-VC-PEG3-azide and endo-BCN-PEG4-PFP ester was conducted using the following final reaction conditions: 2.43 mM oligonucleotide-PAB-VC-PEG3-azide was reacted with 1.62 mM endo-BCN-PEG4-PFP ester (1.5:1 mol:mol ratio) in a 1:1 mixture of DMA and 25 mM MES pH 5.5 buffer. The total pre-reaction volume was 0.17 mL, and the pre-reaction step was allowed to proceed for 4 h at room temperature.

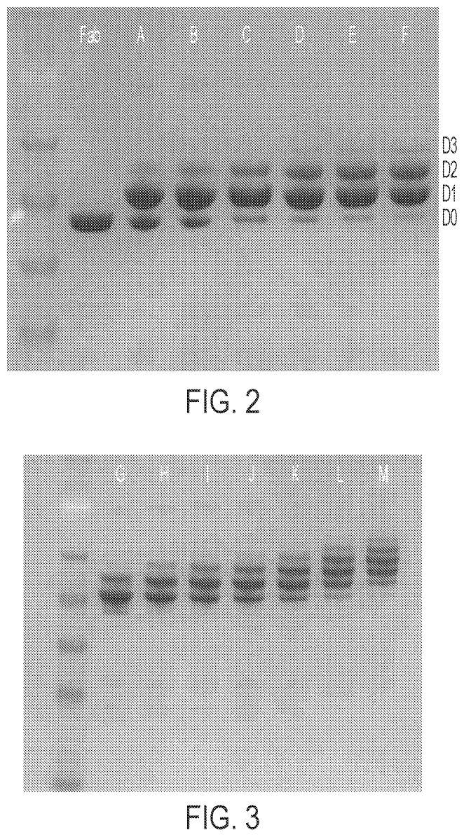

Upon completion of the pre-reaction, the crude (i.e., non-purified) pre-reaction mixture containing oligonucleotide-PAB-VC-PEG3-triazole-PEG4-PFP ester was used to set up a series of anti-TfR Fab conjugation reactions. The independent anti-TfR Fab conjugation reactions were conducted using 1.0, 1.3, 1.6, 1.9, 2.2, and 2.5 molar equivalents of BCN from the pre-reaction product mixture with respect to Fab. All other reaction conditions were held constant across the different conjugations. In the conjugation reaction mixture, the Fab concentration was 6 mg/mL with 1 mg total Fab per reaction and 15 v/v % DMA in 20 mM HEPES pH 7.5 buffer. The reaction was conducted at room temperature for 20 hr. The final average DAR and % unconjugated Fab (D0) for each conjugate were determined by SDS-PAGE densitometry and the results are shown in Table 4. The SDS-PAGE gel is shown in FIG. 2.

TABLE 4

| Rxn | D0 | Avg. DAR |
|---|---|---|
| A | 0.322 | 0.761 |
| B | 0.246 | 0.883 |
| C | 0.156 | 1.055 |
| D | 0.092 | 1.218 |
| E | 0.056 | 1.330 |
| F | 0.053 | 1.431 |

### Example 4: Effect of the Ratio of BCN to Fab and Reaction Conditions on Fab-Oligonucleotide Conjugation Reactions

To investigate the effect of the ratio of BCN from the pre-reaction mixture to anti-TfR, as well as the effect of oligonucleotide and Fab concentrations in oligonucleotide-Fab conjugation reactions, a set of reactions were conducted according to the general protocol described in Example 1.

The pre-reaction between oligonucleotide-PAB-VC-PEG3-azide and endo-BCN-PEG4-PFP ester was conducted using the following reaction conditions: 6.15 mM oligonucleotide-PAB-VC-PEG3-azide was reacted with 6.15 mM endo-BCN-PEG4-PFP ester (1:1 mol:mol ratio) in a mixture of DMA and 25 mM MES pH 5.5 buffer (60:40 DMA:MES), with a total reaction volume of 0.11 mL. The pre-reaction proceeded for 110 minutes at room temperature.

Upon completion of the pre-reaction, the crude (i.e., non-purified) pre-reaction mixture containing oligonucleotide-PAB-VC-PEG3-triazole-PEG4-PFP ester was used to set up a series of anti-TfR Fab conjugation reactions. The independent anti-TfR Fab conjugation reactions were conducted using 2, 3, 4, 5, 6, 8, and 10 molar equivalents of BCN from the pre-reaction product mixture with respect to Fab. The Fab concentration in each reaction was 10 mg/mL with 0.65 mg total Fab per reaction. All other reaction conditions for the conjugations with the exception of DMA content were held constant across the different conjugation reactions. The reactions using 2, 3, 4, 5, and 6 molar equivalents of BCN were conducted in 15 v/v % DMA in 50 mM HEPES pH 7.5 buffer at 23-25° C. for 20 h. The reactions using 8 and 10 molar equivalents of BCN were conducted in 20 v/v % DMA in 50 mM HEPES pH 7.5 buffer at 23-25° C. for 19 h. The final average DAR for each conjugate was determined by SDS-PAGE densitometry, the results of which are shown in Table 5. The SDS-PAGE gel is shown in FIG. 3.

TABLE 5

| Rxn | Avg. DAR |
|---|---|
| G | 1.31 |
| H | 1.63 |
| I | 1.84 |
| J | 1.99 |
| K | 2.38 |
| L | 3.30 |
| M | 3.75 |

### Example 5: Fab-Oligonucleotide Conjugation and Purification

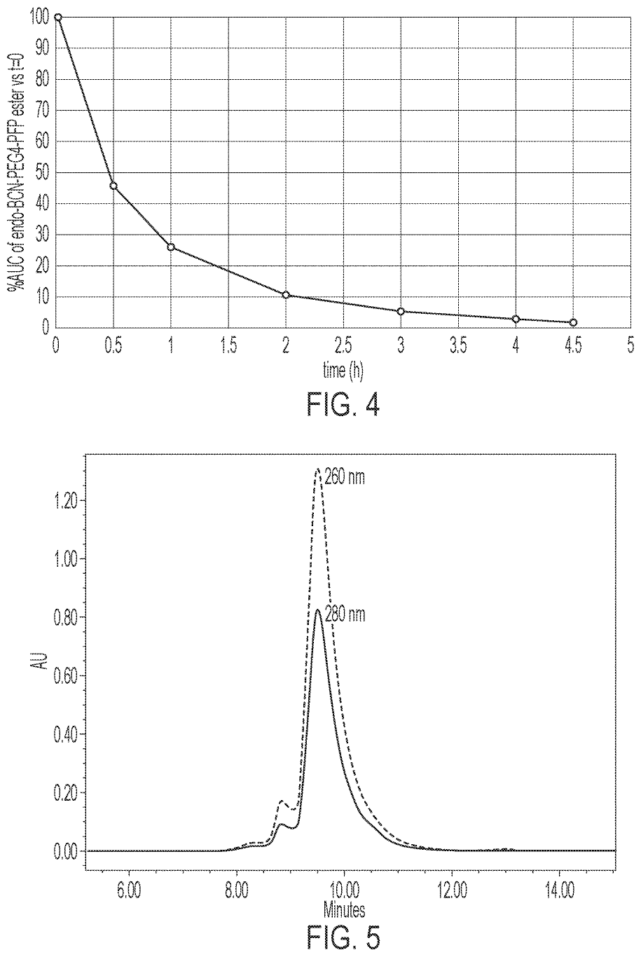

The pre-reaction between oligonucleotide-PAB-VC-PEG3-azide and endo-BCN-PEG4-PFP ester was conducted using the following reaction conditions: 2.61 mM oligonucleotide-PAB-VC-PEG3-azide was reacted with 1.74 mM endo-BCN-PEG4-PFP ester (1.5:1.0 mol:mol equivalents) in a 1:1 v/v % mixture of DMA and 25 mM MES pH 5.5 buffer, with a total reaction volume of 0.44 mL. The pre-reaction proceeded for 4.75 h at room temperature. Completion of the pre-reaction was monitored by measuring the decrease in peak area of the endo-BCN-PEG4-PFP ester starting material by RP C18 UPLC over time (FIG. 4).

Upon completion of the pre-reaction, the crude (i.e., non-purified) pre-reaction mixture containing oligonucleotide-PAB-VC-PEG3-triazole-PEG4-PFP ester was used to set up a series of anti-TfR Fab conjugation reactions. The anti-TfR Fab conjugation was conducted with 2.2:1 mol:mol equivalents of oligonucleotide-PAB-VC-PEG3-triazole-PEG4-PFP ester from the pre-reaction product mixture relative to Fab. The Fab concentration in the conjugation reaction was 6 mg/mL, and the reaction was conducted at a scale of 10 mg total Fab. The final reaction mixture had a composition of 15 v/v % DMA in 20 mM HEPES pH 7.5 buffer.

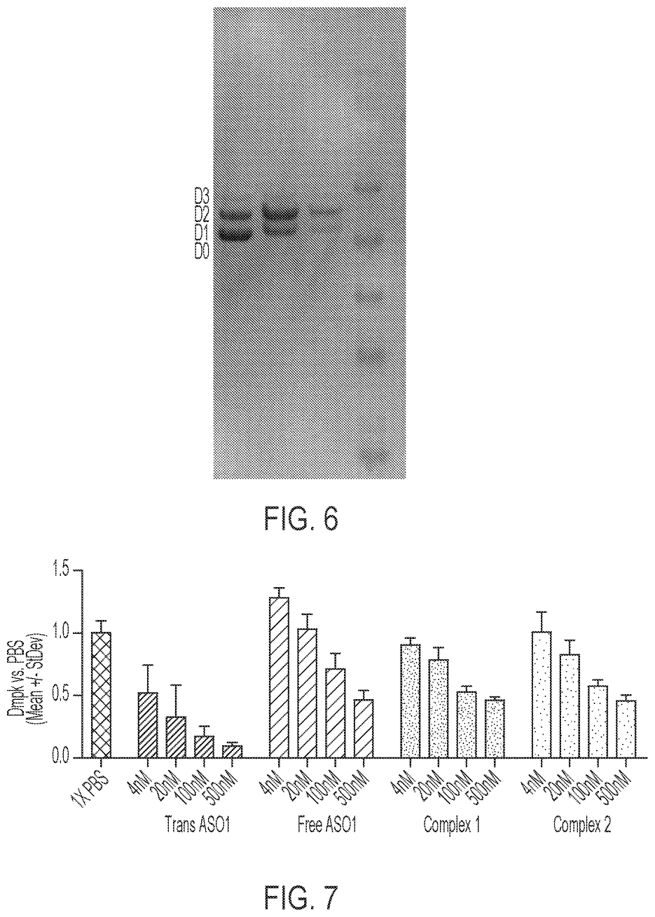

After reacting for 19 h at room temperature, the crude conjugate product mixture was purified by chromatography to remove free oligonucleotide species. First, after loading onto a 1 mL CHT Type I hydroxyapatite column, the conjugate was washed with 5 column volumes (CV) of 100% buffer A (10 mM sodium phosphate and 10 mM sodium chloride, pH 6.5). A second wash was then performed with 10 CV of a 95:5 mixture of buffer A to buffer B (100 mM sodium phosphate and 100 mM sodium chloride, pH 7.5) at a flow rate of 1 mL/min. Finally, a third wash was performed with 10 CV of a 90:10 mixture of buffer A to buffer B at a flow rate of 1 mL/min. After the washing steps, the conjugate was eluted with 100% buffer B at a flow rate of 1 mL/min. This process afforded 7.4 mg (74% yield) of an anti-TfR Fab-oligonucleotide conjugate with an average DAR of 1.30. An SEC chromatogram and an SDS-PAGE gel of the purified anti-TfR Fab-oligonucleotide conjugate are shown in FIG. 5 and FIG. 6, respectively.

### Example 6: Synthesis of a Complex Comprising an Antibody Linked to an Oligonucleotide (Conjugation Method 2—Two Step Conjugation)

A muscle-targeting complex was generated comprising an antisense oligonucleotide covalently linked, via a cathepsin cleavable linker, to an anti-transferrin receptor hIgG1-kappa antibody (anti-TfR antibody).

The anti-TfR antibody was stably expressed in CHO-K1SP cells (Genscript) in a 15 L batch-fed culture. Supernatant was purified over MabSelectSure protein-A affinity resin, washed with 20 mM phosphate and eluted with 50 mM sodium citrate at pH=3.5. The elution was neutralized with 1.0 M NaOH and further polished by size exclusion chromatography (SEC) into DPBS pH=7.4 and concentrated via tangential flow filtration (TFF) to 10.0 mg/mL.

The purified antibody was site specifically cleaved (at sequence of CPAPELLG-GPSVF (SEQ ID NO: 45)) to F(ab′)2 fragments using FragIt solid supported IdeS enzyme (1.0 gAb/10 mL resin, 120 minutes at room temperature in shake flask at 125 rpm). FC domain and any uncut IgG was removed using CaptureSelect FcXL (ThermoScientific) with binding capacity of 25 mg/mL on HiLoad column (26 mm×40 cm, [Load flow rate: linear 113 cm/hr with 26 mm ID-10.0 ml/min volumetric flow, residence time-21.2 minutes]). Flow through was collected containing purified F(ab′)2 and verified by SDS-PAGE (4-12% NuPAGE, MES pH=7.0, 160v, 40 min) and analytical HPLC-SEC (Zorbax GF-250, 9.4 mm×250 mm, PBS pH=7.2, 25 C) for any breakthrough. The F(ab′)2 fragments were reduced to Fab′ with 80-molar excess of cysteamine-HCl (ChemImpex #02839) per hinge thiol at 37° C. for 90 min. Fab′ was then immediately purified away from non-reduced F(ab′)2 and free cysteamine with protein L chromatography (GE #17547855, 10× series) using a standard pH gradient (50 mM Na3C6H5O7, pH=2.6, 60-100% gradient over 20 column volumes). Anti-TfR Fab can also be recombinantly produced.

Anti-TfR Fab was diluted 1:10 v/v with acetonitrile (HPLC grade), and reacted with a 5-molar excess of endo-BCN-PEG3-PFP ((endo) bicyclononyne-PEG3-pentafluorophenyl (Formula (C)); dissolved at 20 mg/mL in DMSO) for 2 hours at room temperature (˜22.5° C.). Following the reaction, the anti-TfR Fab BCN solution was sterile filtered or depth filtered to remove precipitated BCN. The filtered solution was then assayed for average reactive BCN moieties using LCMS and azide-A488 (20 molar excess) for 90 minutes. Fab that incorporated BCN (>1.3 moles of BCN per mole of Fab) was purified with 10 kDa-TFF (1.2 bar, Sartorius VivaFlow 200) with 5 filtrate volumes to remove free BCN. Complete removal of free BCN was verified by analytical HPLC-SEC (Tosoh, TSKgel SuperSW mAb HR, 7.8 mm×300 mm). Recovery of BCN-modified anti-TfR Fab was >90% of starting material. The purified Fab that incorporated BCN was carried to the next step.

A linker/payload compound comprising the oligonucleotide (e.g., charged oligonucleotide) and an azide-Valine-Citrulline linker was generated. The oligonucleotide (Na+ adduct) was dissolved at 200 mg/mL in RNAse free water. This solution was diluted to 10 mg/mL with dry dimethylformamide (DMF). A 25-fold molar excess of tributylamine was then added to the solution. The linker molecule (azide-PEG3-Val-Cit-PAB-PNP (Formula (A)), dissolved at 20 mg/mL in DMF) was added at a 2-fold molar excess to the oligonucleotide solution for 120 min at ˜25° C. Reaction completion was measured using ninhydrin (Kaiser test) prior to quenching the reaction using an alcohol precipitation. The alcohol precipitation was accomplished by addition of 0.1 v/v 3 M NaCl solution, followed by addition of 3 volumes of −80° C. isopropanol. The solution was then thoroughly mixed and subsequently allowed to precipitate at −20° C. for 1 hour. The precipitated solution was centrifuged (at 4300× g; 8° C.) for 30 mins and the solvent was decanted. The pellet was washed with −80° C. 80% Ethanol in RNase free water (one volume equivalent to starting reaction) and centrifuged (at 4300× g; 8° C.) for 20 min. Ethanol was then decanted and the pellet (containing the compound comprising the oligonucleotide and an azide-Valine-Citrulline linker) was dried for 10 min at 37° C. The linker/payload compound comprising the oligonucleotide and azide-Valine-Citrulline linker was resuspended in 20% Acetonitrile in Nuclease free water to a concentration of 20 mg/mL.

The BCN-modified anti-TfR Fab was reacted with 2.5 molar equivalents per BCN of the linker/payload compound comprising the oligonucleotide and the azide-Valine-Citrulline linker for 2 hours at room temperature (˜25° C.).

The completion of the coupling reaction was evaluated by SDS-PAGE and analytical SEC, which demonstrated a 78% coupling efficiency by densitometry.

The biological activity of the conjugates generated by the conjugation method described in Example 1 and the conjugates generated by the conjugation method described in Example 6 were tested. Both conjugates contain a DMPK-targeting oligonucleotide (ASO1) and efficiently knocked down DMPK mRNA level in RD cells (FIG. 7).

### Example 7: Synthesis of a Complex Comprising an Antibody Linked to a Charge-Neutral Oligonucleotide (Conjugation Method 2—Two Step Conjugation)

A muscle-targeting complex was generated comprising an oligonucleotide (e.g., a charge-neutral oligonucleotide) covalently linked, via a cathepsin cleavable linker, to an anti-transferrin (anti-TfR) receptor Fab antibody. Anti-TfR Fabs can be recombinantly produced (e.g., in CHO cells) and purified. The oligonucleotide used is a phosphorodiamidate morpholino oligomer (PMO) that is 30 nucleotides in length.

The anti-TfR Fab was diluted with propylene glycol to a final concentration of 40% v/v propylene glycol and incubated with 5-fold molar excess of endo-BCN-PEG3-PFP (endo-bicyclononyne-PEG3-pentafluorophenyl, dissolved in DMSO at a concentration of 20 mg/mL] for 2 hr at room temperature (˜22.5° C.). It was anticipated that labeling should yield 2.0-2.5 moles of BCN per mole of Fab. Post labeling, the reaction product was sterile filtered or depth filtered to remove precipitated BCN. The filtered solution was then assayed for average reactive BCN moieties analytically using LCMS (ThermoFisher MAbPac RP 4 um 2.1×100 mm, #088647; mobile phase A 0.1% formic acid in 100% UPLC-grade water, mobile phase B 0.1% formic acid in 100% UPLC-grade acetonitrile; flow rate 0.3 mL/min; column temperature 70° C.; in-source CID 20 eV; positive polarity; spray voltage 3.5 kV; scan range 1000-3000 m/z).

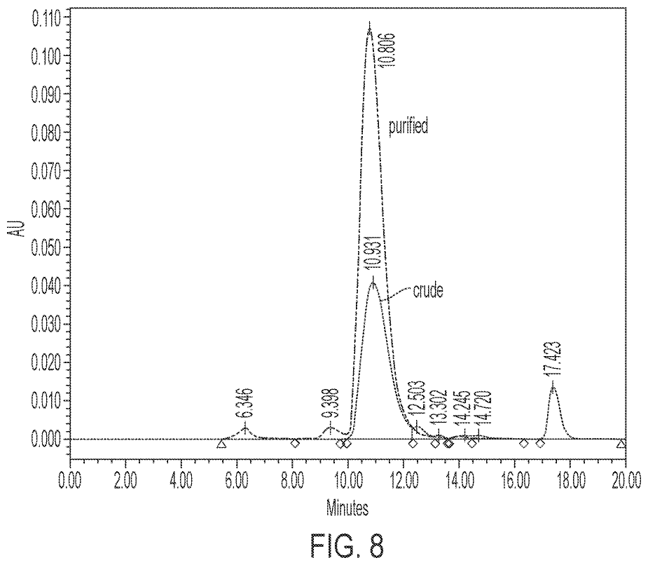

Anti-TfR having a degree of labeling (D0L) of >2.3 was taken to the next step of the conjugation, and was purified into 10% isopropanol in PBS at pH 7.2 by tangential flow filtration using a 10 kDa molecular weight cutoff (1.2 bar), with 5 filtrate volumes, to remove free BCN and propylene glycol. Complete removal of BCN and propylene glycol was verified by analytical HPLC-SEC (Waters Xbridge Protein BEH SEC 3.5 um, 7.8×300 mm, 0.3 mL/min, 100 mM PO₄, 100 mM NaCl, 15% v/v acetonitrile pH 7.0). SEC traces of the crude and purified products are shown in FIG. 8. The recovery of BCN-labeled anti-TfR was >90% of starting material. The purified solution was concentrated to 3.5 mg/mL for further conjugation steps.

In a separate reaction, the oligonucleotide (e.g., a charge-neutral oligonucleotide) was conjugated to a linker molecule. The oligonucleotide was dissolved at 35 mg/mL in anhydrous DMSO at 37° C. The linker molecule (azide-PEG3-Val-Cit-PAB-PNP) was dissolved at 40 mg/mL in anhydrous DMF and was added at 2.7-fold molar excess to the oligonucleotide with 3-fold molar excess of N,N-Diisopropylethylamine (DIPEA). This linker conjugation reaction was allowed to proceed for 2 hours at room temperature (˜22.5° C.). Progress and completion of the reaction was measured using a ninhydrin assay (Kaiser test) prior to quenching the reaction via acetone precipitation.

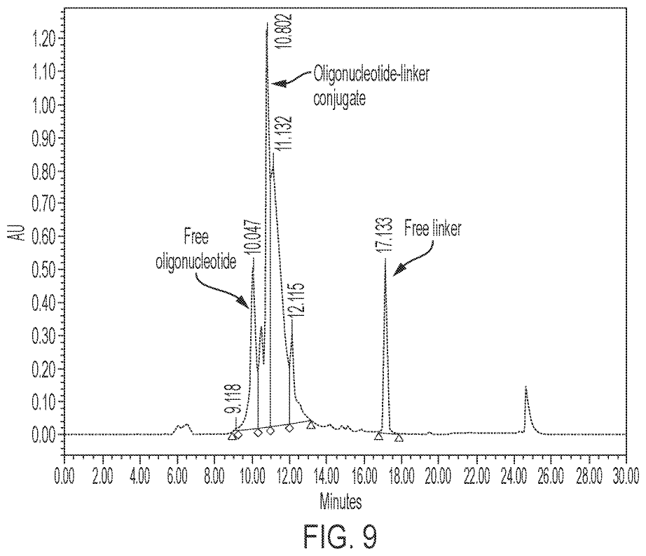

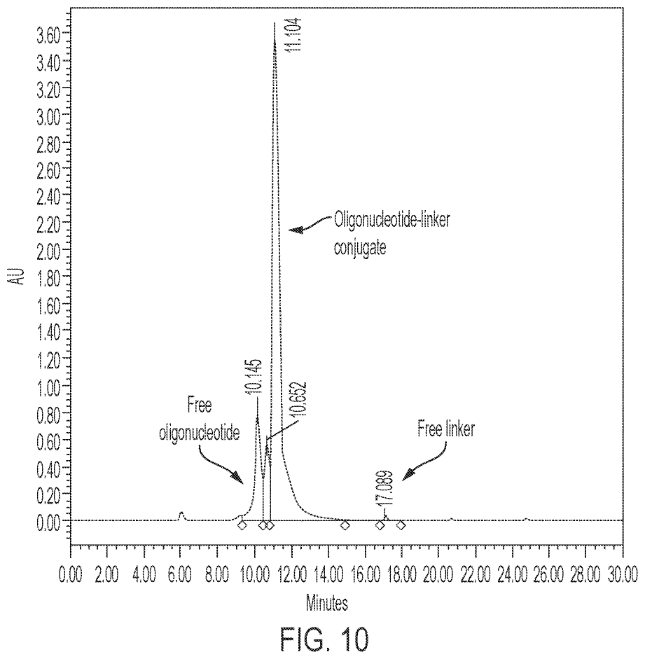

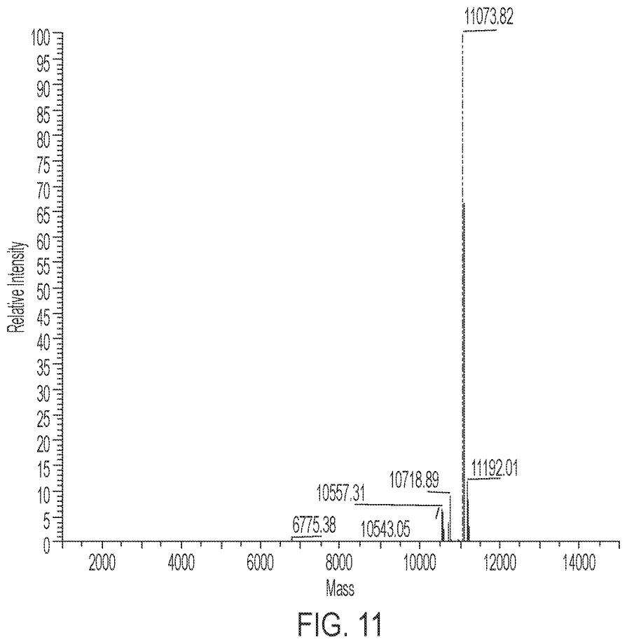

Precipitation was conducted by adding 8 volumes of chilled acetone to the product solution, and the precipitate was pelleted by centrifugation at 3500×g at 8° C. for 20 minutes. The pellet was then washed with 3 volumes of acetone to remove remaining free linker and was centrifuged again at 3500× g at 8° C. for 20 minutes. The purified oligonucleotide-linker was then dissolved in 20% v/v acetonitrile in nuclease-free water at a concentration of 30 mg/mL. The concentration and yield were measured by optical density (OD) in 0.1N HCl, demonstrating a yield of greater than 90%. Analytical RP-HPLC was conducted (Waters BEH-C18, 4.6 mm×150 mm, 0.5 mL/min. 5-90% v/v acetonitrile in water, 30 minute run time) on the crude linker/oligonucleotide conjugation reaction product (FIG. 9) and the purified oligonucleotide-linker (FIG. 10) to confirm removal of free linker by the precipitation and washing steps. Confirmatory LCMS of the purified oligonucleotide-linker was also conducted (FIG. 11).

To conjugate the anti-TfR and the oligonucleotide, BCN-labeled antibody was mixed with 5-fold molar excess of oligonucleotide-PAB-VC-PEG3-azide (FIG. 1A) in a glass bottle overnight at room temperature (˜22.5° C.).

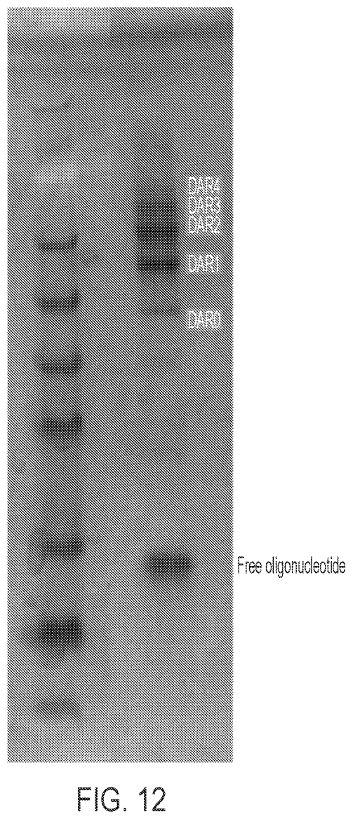

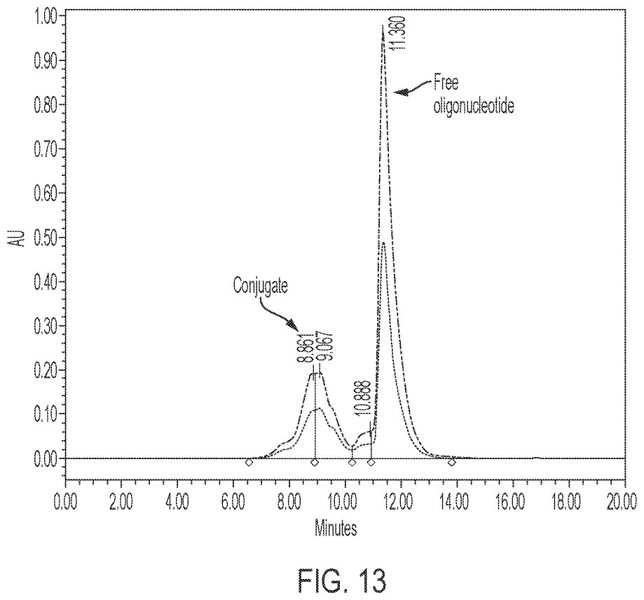

Completion of the reaction was evaluated by SDS-PAGE (FIG. 12) and analytical SEC analysis (FIG. 13), which demonstrated less than 10% unlinked anti-TfR antibody (DAR0) and a 90% coupling efficiency by densitometry.

### Example 8. Conjugation Process for Preparation of a Fab-Oligonucleotide (Charge-Neutral Oligonucleotide) Conjugate

### (Conjugation Method 1—Pre-Reaction Conjugation)

This example describes the preparation of a conjugate composed of an oligonucleotide covalently linked via a val-cit cathepsin cleavable peptide linker to an anti-transferrin receptor (anti-TfR) Fab antibody. Anti-TfR Fabs can be recombinantly produced (e.g., in CHO cells) and purified. The oligonucleotide used is a phosphorodiamidate morpholino oligomer (PMO) that is 30 nucleotides in length. Prior to the conjugation with the Fab, an intermediate containing the oligonucleotide and the linker is generated via a copper-free 3+2 click reaction between the azide group of an oligonucleotide-PAB-VC-PEG3-azide molecule (FIG. 1A) and the strained bicyclononyne moiety on an endo-BCN-PEG4-PFP ester (FIG. 1B) heterobifunctional crosslinker (“the pre-reaction”). Lyophilized oligonucleotide-PAB-VC-PEG3-azide (98.1 mg) was solubilized in a 4 mL glass Wheaton vial in 0.32 mL of MilliQ water. Following solubilization, 0.32 mL of N,N-dimethylacetamide (DMA) was added and the mixture was gently agitated for 5-10 minutes. Prior to continuing, the vial was inspected carefully to ensure the oligonucleotide-PAB-VC-PEG3-azide was completely dissolved and no residue remained on the walls of the glass vial. The concentration of the oligonucleotide-PAB-VC-PEG3-azide stock solution in 1:1 DMA:water was determined with a Nanodrop UV/vis instrument by using aliquots diluted 25-, 50-, and 100-fold in 1:1 DMA:water containing a final concentration of 0.1 M HCl at 265 nm, using an extinction coefficient of 318,050 M⁻¹ cm⁻¹. The HCl was added to ensure accuracy of the concentration measurement. The calculated concentration at each dilution was averaged to determine the solution concentration of 10.1 mM.

A 32.5 mg/mL (53.5 mM) stock solution of endo-BCN-PEG4-PFP ester was prepared by weighing approximately 25 mg of endo-BCN-PEG4-PFP ester oil into a 4 mL glass Wheaton vial. The appropriate volume of DMA was then added to afford the 32.5 mg/mL stock solution.

The pre-reaction was conducted with the following final solution reaction conditions: 5.87 μM (6.5 μmol) oligonucleotide-PAB-VC-PEG3-azide, 5.34 mM endo-BCN-PEG4-PFP ester (1.1:1.0 mol:mol equivalents) in 60:40 v/v % DMA to 25 mM 2-(N-morpholino)ethanesulfonic acid (MES) pH 5.5 buffer at room temperature. The reaction was set-up in a 4 mL glass Wheaton vial by adding the appropriate amounts of the reactants and stock solutions as indicated in Table 6. The total final volume of the pre-reaction was 1.11 mL.

TABLE 6

| Addition | | Stock | | Volume |
|---|---|---|---|---|
| Order | Solution | Concentration | | (mL) |
| 1 | MES, pH 5.5 buffer stock | 500 | mM | 0.0222 |
| 2 | MilliQ Water | NA | | 0.0981 |
| 3 | DMA | NA | | 0.2313 |
| 4 | oligonucleotide-PAB-VC-PEG3-azide | 10.1 | mM | 0.6456 |
| | stock in 1:1 | | | |
| | DMA:MilliQ water | | | |
| 5 | Endo-BCN-PEG4-PFP ester stock in DMA | 53.5 | mM | 0.1105 |

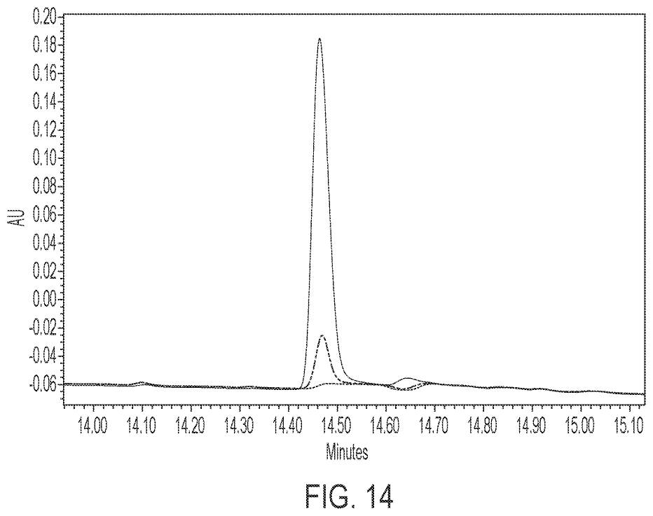

Completion of the pre-reaction to generate oligonucleotide-PAB-VC-PEG3-triazole-PEG4-PFP ester (FIG. 1C) was monitored by RP-C18 UPLC with repeat injections every 30 minutes (at 5 minute, 35 minute, and 65 minute time points), by observing the disappearance of the endo-BCN-PEG4-PFP ester starting material at 220 nm. This IPC indicated the reaction was complete at 65 minutes (FIG. 14). The reaction was determined to be complete when less than 5% of the endo-BCN-PEG4-PFP ester was remaining with respect to the 5 minute time point (Table 7). The crude pre-reaction mixture was carried forward to the conjugation reaction immediately without any purification. The total pre-reaction time was 90 minutes defined as the time between addition of the endo-BCN-PEG4-PFP ester to the pre-reaction and addition of this reaction mixture to the Fab conjugation reaction.

TABLE 7
Quantification of endo-BCN-PEG4-PFP ester starting
material based on RP-C18 UPLC measurements.

| | % endo-BCN-PEG4-PFP |
|---|---|
| Time (min) | ester remaining |
| 5 | 100 |
| 35 | 13.6 |
| 65 | 2.3 |

Conjugation of the oligonucleotide to the anti-TfR Fab involves the formation of an amide bond between solvent accessible lysine residues of the Fab and the activated PFP ester of the oligonucleotide-PAB-VC-PEG3-triazole-PEG4-PFP ester (FIG. 1C) that was generated in the pre-reaction step.

Prior to setup of the conjugation reaction, the anti-TfR Fab formulated in 20 mM sodium citrate, 100 mM sodium chloride was buffer exchanged into 50 mM HEPES pH 7.5. Anti-TfR Fab (10 mL at 10.15 mg/mL) was loaded onto 50 mM HEPES pH 7.5 equilibrated NAP-25 desalting columns (4×2.5 mL) and eluted with 50 mM HEPES pH 7.5 (4×3.5 mL). The eluate was pooled and concentrated with an Amicon Ultra-15 10 kDa centrifugal filter unit spinning at 4000 rcf to reduce the volume to 2.86 mL. The concentration of the buffer resultant anti-TfR Fab was measured by Nanodrop UV/vis to be 31.75 mg/mL.

The conjugation reaction was conducted with the following final solution reactant amounts: anti-TfR Fab (45 mg, 6 mg/mL, 125 μM) and oligonucleotide-PAB-VC-PEG3-triazole-PEG4-PFP ester at a final theoretical concentration of 750 μM (6.0:1.0 mol:mol equivalents of oligonucleotide-PAB-VC-PEG3-triazole-PEG4-PFP ester vs anti-TfR Fab). The oligonucleotide-PAB-VC-PEG3-triazole-PEG4-PFP ester concentration assumed 100% conversion of the BCN in the pre-reaction into oligonucleotide-PAB-VC-PEG3-triazole-PEG4-PFP ester. The final reaction mixture consisted of 15:85 v/v % DMA to 25 mM HEPES pH 7.5 buffer. The reaction was set-up in a 20 mL glass scintillation vial by adding the appropriate amounts of the reactants and stock solutions as indicated in Table 8. The reaction proceeded for 20 h at room temperature (˜25° C.). The start of the reaction was defined as the addition time of the pre-reaction mixture containing the oligonucleotide-PAB-VC-PEG3-triazole-PEG4-PFP ester to the anti-TfR Fab solution. The total duration of the conjugation reaction was 20 hours.

TABLE 8

| Addition | | Stock | | Volume |
|---|---|---|---|---|
| Order | Solution | Concentration | | (mL) |
| 1 | HEPES, pH 7.5 buffer stock | 500 | mM | 0.177 |
| 2 | MilliQ Water | — | | 4.360 |
| 3 | DMA | — | | 0.492 |
| 4 | Anti-TfR Fab (31.75 mg/mL in 50 | 662 | μM | 1.417 |
| | mM HEPES, pH 7.5) | | | |
| 5 | Pre-reaction mixture | 5.34 | mM | 1.055 |
| | (oligonucleotide-PAB-VC- | | | |
| | PEG3-triazole-PEG4-PFP ester) | | | |

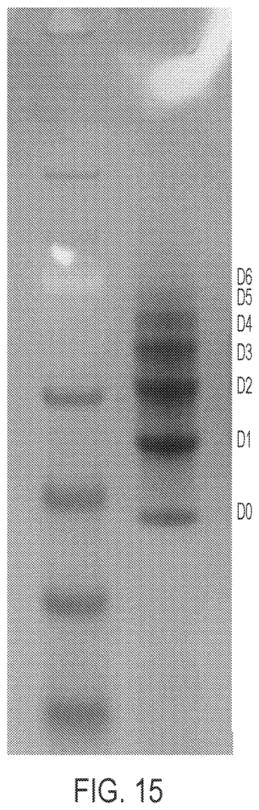

After reacting for 20 hours, the crude conjugate mixture was tested by SDS-PAGE and analyzed by densitometry to determine the drug to antibody ratio (DAR) and % unconjugated Fab. Results are presented in FIG. 15 and Table 9.

TABLE 9

| Species | Abundance |
|---|---|
| D0 | 0.090 |
| D1 | 0.290 |
| D2 | 0.333 |
| D3 | 0.200 |
| D4 | 0.061 |
| D5 | 0.020 |
| D6 | 0.006 |
| Average DAR | 1.93 |

### Example 9. Purification of Anti-TfR Fab-Oligonucleotide Conjugate

Following synthesis of the anti-TfR Fab-oligonucleotide conjugate described in Example 8, a two-part purification process was conducted. First, free payload was removed by hydroxyapatite (HA) chromatography. The HA eluate was then buffer exchanged into the final formulation. At the 45 mg scale of Fab, the final buffer exchange was performed with a 30 kDa centrifugal filter device. Prior to loading onto the HA column, the crude reaction product from Example 8 (anti-TfR Fab-oligonucleotide conjugate) was diluted and the pH adjusted from 7.5 to 5.7. First, the 7.5 mL of crude conjugate was diluted by addition of 16.5 mL of 15 v/v % DMA in water and the solution was thoroughly mixed. To this mixture, 0.75 mL of 500 mM MES (pH 3.3) was added to adjust the pH down to 5.7.

Chromatographic purification to remove unreacted oligonucleotide species was performed using a 5 mL Bio Rad CHT Type I (ceramic hydroxyapatite) cartridge on an AKTA Pure chromatography system. Prior to loading the diluted conjugate pool from the reaction mixture preparation step, the CHT cartridge was prepared and equilibrated according to the manufacturer's instructions using 15:85 v/v % of DMA to 10 mM sodium phosphate, pH 5.8 buffer. Following equilibration, the conjugate pool was loaded at a flow rate of 5 mL/min. After loading the conjugate, the column was washed for a minimum of 7 CV with 15:85 v/v % of DMA in 10 mM sodium phosphate buffer (pH 5.8). After completion of the wash, elution was initiated via a step gradient with 100 mM sodium phosphate, pH 7.6 buffer containing DMA at 15:85 v/v % at a flow rate of 5 mL/min. The entire elution peak, identified by monitoring at 260 nm and 280 nm, was collected and pooled.

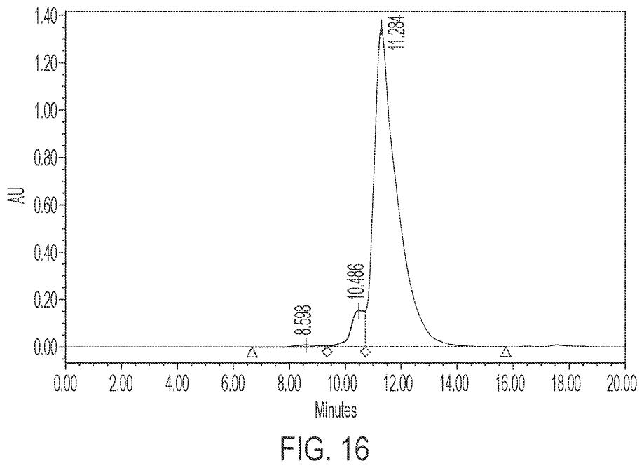

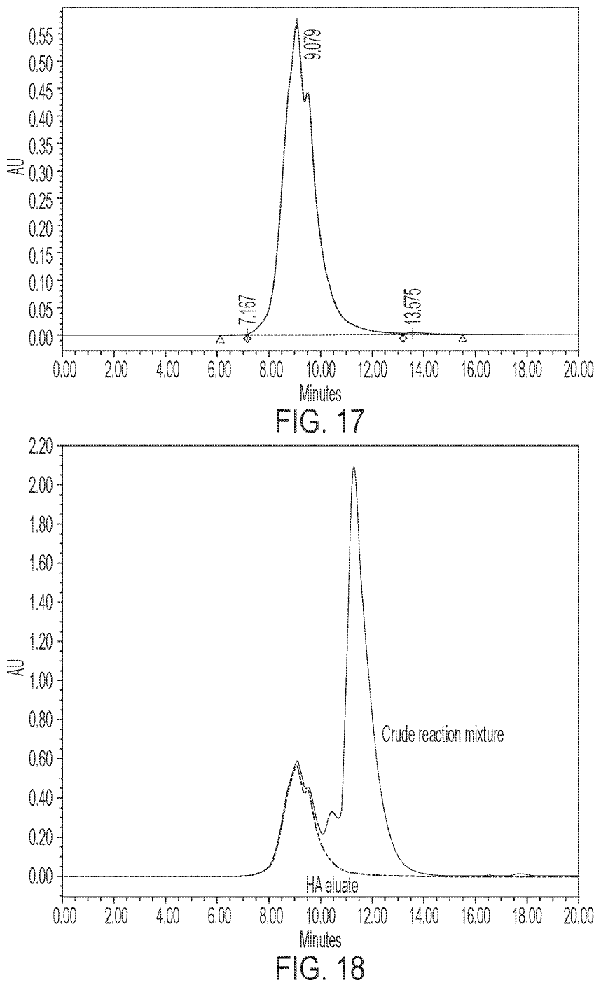

Analysis of the flow through during the HA column loading step by SEC (FIG. 16) indicated the presence of little to no Fab-oligonucleotide conjugate in the flow-through. Only peaks due to oligonucleotide payload species were observed, at ˜10.5 and ˜11.3 minutes. Conversely, SEC analysis of the pooled elution peak shows only conjugate species (FIG. 17), with multiple peaks and shoulders due to the size differences of the conjugates with different oligonucleotide (e.g., PMO) payload loadings. No peaks for payload species at 10.5 or 11.3 minutes were observed. The conjugate mass balance with respect to Fab for the HA purification was estimated by SEC chromatography. This was accomplished by injecting 24 ug of Fab from the crude conjugation reaction product (4 μL injection at 6 μg/mL concentration) and injecting a theoretical 24 μg of Fab, assuming 100% recovery, from HA eluate pool. The HA eluate pool was 13.9 mL theoretically containing 45 mg of Fab, giving a theoretical concentration of 3.24 μg/μL. To achieve an injection of 24 μg of Fab, a 7.4 μL injection was used. Overlay of these two SEC chromatograms (FIG. 18) indicates nearly complete recovery of the Fab as Fab-oligonucleotide conjugate. The peak height at 9.1 min was used to estimate a 97% recovery of the reaction product after the HA chromatographic purification.

At the 45 mg reaction scale, buffer exchange of the HA eluate into 50 mM His (pH 6.0) was performed using an Amicon Ultra-15 30 kDa centrifugal filtration device. First, the HA eluate pool was concentrated to approximately 1.5 mL by spinning the column at 4000 rcf. Buffer exchange was subsequently performed via addition of 3 mL of 50 mM His (pH 6.0) and concentration by centrifugation at 4000 rcf until the volume reached approximately 1.5 mL. This step was repeated for five total rounds, using the equivalent of 15 volumes of buffer to generate the final purified anti-TfR Fab-oligonucleotide conjugate. The resulting ˜1.5 mL of purified conjugate (in 50 mM His, pH 6.0) was then diluted to a final volume of 3.0 mL with additional 50 mM His (pH 6.0).

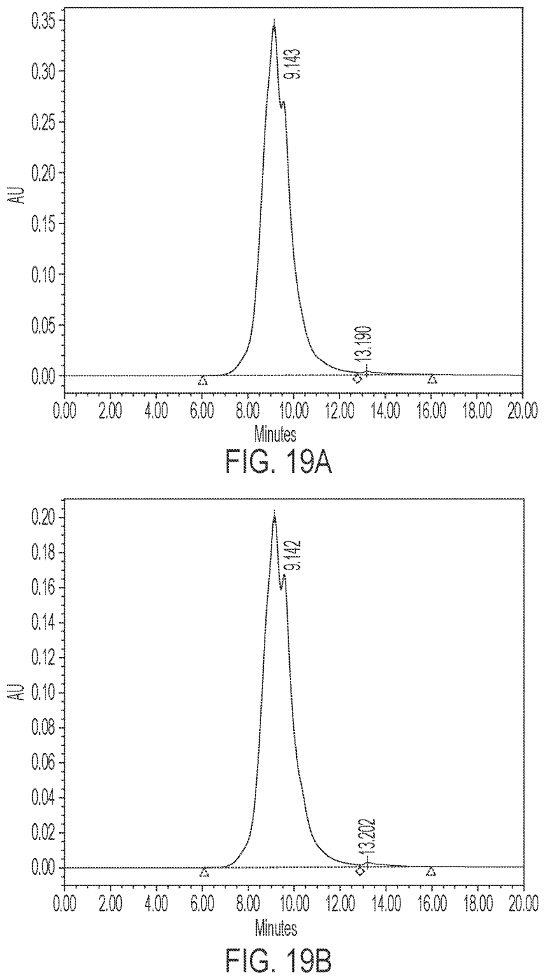

The final purified anti-TfR Fab-oligonucleotide conjugate was analyzed by SEC, SDS-PAGE densitometry, and BCA. SEC of the final conjugate (shown in FIGS. 19A and 19B) was nearly identical to the corresponding SEC data of the conjugate pool after HA purification, indicating the purification process did not induce formation of high molecular weight species. The average DAR, DAR species distribution, and percent unconjugated Fab were calculated by SDS-PAGE densitometry (SDS-PAGE gel shown in FIG. 20) and data analysis was performed with the Image Studio Lite software package from Li-Cor Biosciences (calculation results shown in Table 10). The final average DAR of the purified anti-TfR Fab-oligonucleotide conjugate was 1.96, including 8.1% unconjugated anti-TfR Fab. Protein concentration was measured to be 10.5 mg/mL by the BCA assay, indicating a total of 31.4 mg of conjugate in the final product, for an overall process yield of 70%.

TABLE 10
Average DAR and DAR distribution of the purified
Fab-oligonucleotide conjugate product.

| Species | Abundance |
|---|---|
| D0 | 0.081 |
| D1 | 0.324 |
| D2 | 0.312 |
| D3 | 0.168 |
| D4 | 0.071 |
| D5 | 0.032 |
| D6 | 0.011 |
| Average DAR | 1.96 |

### Example 10. Effect of the Ratio of BCN to Fab and Reaction Conditions on Fab-Oligonucleotide Conjugation Reactions

To investigate the effect of the ratio of BCN from the pre-reaction mixture to anti-TfR, as well as the effect of oligonucleotide and Fab concentrations in oligonucleotide-Fab conjugation reactions, a set of reactions were conducted according to the general protocol described in Example 8. The oligonucleotide used is a PMO of 30 nucleotides in length.

In a first set of reactions, the pre-reaction between oligonucleotide-PAB-VC-PEG3-azide and endo-BCN-PEG4-PFP ester was conducted using the following conditions: 2.44 mM oligonucleotide-PAB-VC-PEG3-azide with 1.63 mM endo-BCN-PEG4-PFP ester (1.5:1 mol:mol ratio) were reacted in a 1:1 mixture of DMA and 25 mM MES pH 5.5 buffer. The total pre-reaction volume was 0.37 mL, and the pre-reaction step was allowed to proceed for 18 h at room temperature (˜25° C.).

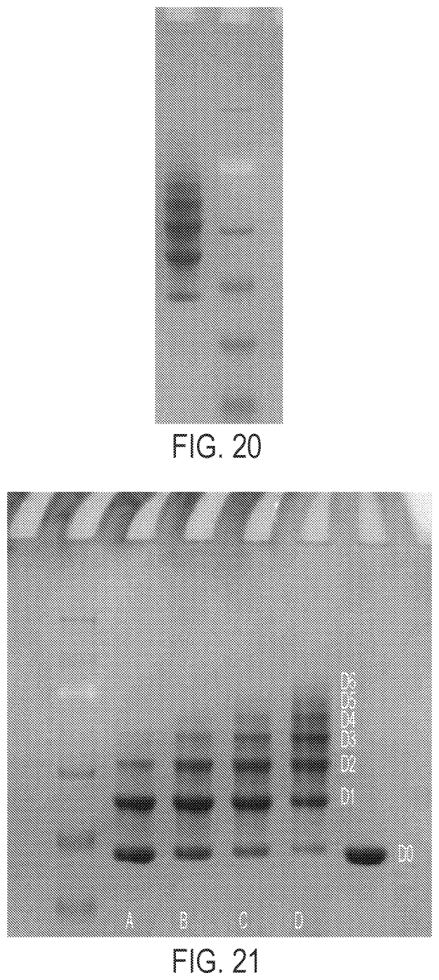

Upon completion of the pre-reaction, the crude (i.e., non-purified) pre-reaction mixture containing oligonucleotide-PAB-VC-PEG3-triazole-PEG4-PFP ester was used to set-up a series of anti-TfR Fab conjugation reactions. The anti-TfR Fab conjugation reactions were conducted using 2, 4, 6, and 10 molar equivalents of BCN from the pre-reaction mixture with respect to Fab. All other reaction conditions were held constant across the different conjugations. In the conjugation reaction mixture, the Fab concentration was 3 mg/mL with 1 mg total Fab per reaction in 15 v/v % DMA in 50 mM HEPES pH 7.5 buffer. The conjugation reaction was conducted at 23-25° C. for 18 h. The final DAR and DAR species distributions for each conjugate were determined by SDS-PAGE densitometry and the results are shown in Table 11. An image of the SDS-PAGE gel is shown in FIG. 21.

TABLE 11

| Reac- | BCN | Peak Fraction | | | | | | | Avg. |
|---|---|---|---|---|---|---|---|---|---|
| tion | equiv. | D0 | D1 | D2 | D3 | D4 | D5 | D6 | DAR |
| A | 2 | 0.389 | 0.412 | 0.159 | 0.040 | ND | ND | ND | 0.85 |
| B | 4 | 0.232 | 0.391 | 0.256 | 0.095 | 0.027 | ND | ND | 1.30 |
| C | 6 | 0.150 | 0.361 | 0.308 | 0.117 | 0.053 | 0.012 | ND | 1.60 |
| D | 10 | 0.084 | 0.293 | 0.342 | 0.137 | 0.077 | 0.044 | 0.024 | 2.06 |

In a second set of reactions, the pre-reaction between oligonucleotide-PAB-VC-PEG3-azide and endo-BCN-PEG4-PFP ester was conducted using the following conditions: 6.77 mM oligonucleotide-PAB-VC-PEG3-azide with 4.84 mM endo-BCN-PEG4-PFP ester (1.4:1 mol:mol ratio) were reacted in a 1:1 mixture of DMA and 25 mM MES pH 5.5 buffer. The total pre-reaction volume was 0.160 mL, and the pre-reaction step was allowed to proceed for 90 minutes at room temperature (˜25° C.).

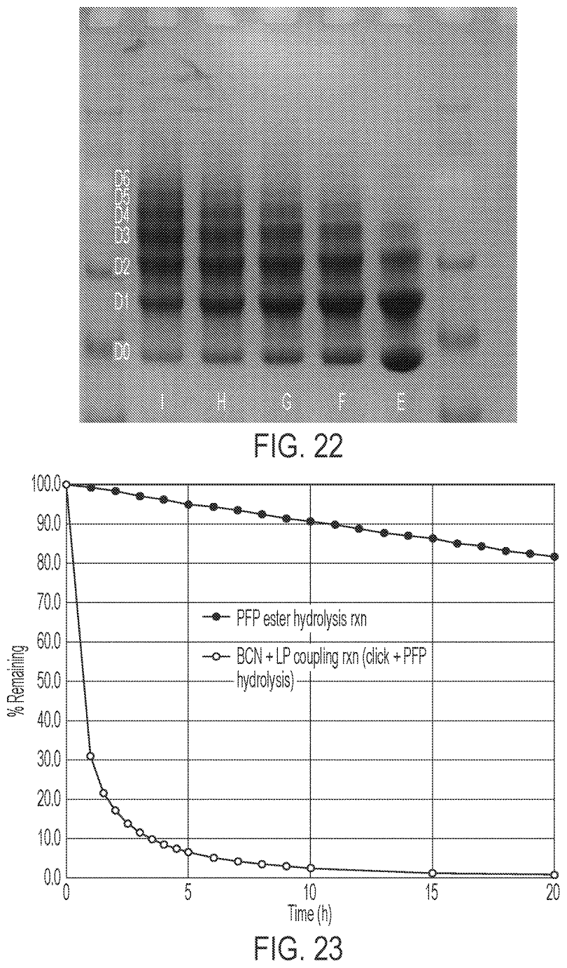

Upon completion of the pre-reaction, the crude (i.e., non-purified) pre-reaction mixture containing oligonucleotide-PAB-VC-PEG3-triazole-PEG4-PFP ester was used to set-up a series of anti-TfR Fab conjugation reactions. The anti-TfR Fab conjugation reactions were conducted using 2, 4, 5, 6, and 8 molar equivalents of BCN from the pre-reaction mixture with respect to Fab. All other reaction conditions were held constant across the different conjugations. In the conjugation reaction mixture, the Fab concentration was 6 mg/mL with 1 mg total Fab per reaction in 15 v/v % DMA in 50 mM HEPES pH 7.5 buffer. The conjugation reaction was conducted at 23-25° C. for 19 h. The final average DAR for each conjugate was determined by SDS-PAGE densitometry and the results are shown in Table 12. An image of the SDS-PAGE gel is shown in FIG. 22.

TABLE 12

| | BCN | Avg. |
|---|---|---|
| Reaction | equiv. | DAR |
| E | 2 | 1.14 |
| F | 4 | 1.64 |
| G | 5 | 1.93 |
| H | 6 | 2.06 |
| I | 8 | 2.21 |

In a third set of reactions, the pre-reaction between oligonucleotide-PAB-VC-PEG3-azide and endo-BCN-PEG4-PFP ester was conducted using the following conditions: 6.77 mM oligonucleotide-PAB-VC-PEG3-azide with 4.84 mM endo-BCN-PEG4-PFP ester (1.4:1 mol:mol ratio) were reacted in a 60:40 mixture of DMA and 25 mM MES pH 5.5 buffer. The total pre-reaction volume was 0.160 mL, and the pre-reaction step was allowed to proceed for 90 minutes at room temperature (˜25° C.).

Upon completion of the pre-reaction, the crude (i.e., non-purified) pre-reaction mixture containing oligonucleotide-PAB-VC-PEG3-triazole-PEG4-PFP ester was used to set-up a series of anti-TfR Fab conjugation reactions. The anti-TfR Fab conjugation reactions were conducted using 2, 4, 5, 6, 8, and 10 molar equivalents of BCN from the pre-reaction mixture with respect to Fab. All other reaction conditions were held constant across the different conjugations. In the conjugation reaction mixture, the Fab concentration was 6 mg/mL with 1 mg total Fab per reaction in 15 v/v % DMA in 50 mM HEPES pH 7.5 buffer. The conjugation reaction was conducted at room temperature (˜25° C.) for 19 h. The final average DAR and percentage of DAR0 for each conjugate was determined by SDS-PAGE densitometry and the results are shown in Table 13 (reactions J, K, L, M, N, O).

In a fourth set of reactions, the pre-reaction between oligonucleotide-PAB-VC-PEG3-azide and endo-BCN-PEG4-PFP ester was conducted using the following conditions: 6.77 mM oligonucleotide-PAB-VC-PEG3-azide with 6.45 mM endo-BCN-PEG4-PFP ester (1.05:1 mol:mol ratio) were reacted in a 60:40 mixture of DMA and 25 mM MES pH 5.5 buffer. The total pre-reaction volume was 0.080 mL, and the pre-reaction step was allowed to proceed for 160 minutes at room temperature (˜25° C.).

Upon completion of the pre-reaction, the crude (i.e., non-purified) pre-reaction mixture containing oligonucleotide-PAB-VC-PEG3-triazole-PEG4-PFP ester was used to set-up a series of anti-TfR Fab conjugation reactions. The anti-TfR Fab conjugation reactions were conducted using 5, 6, and 7 molar equivalents of BCN from the pre-reaction mixture with respect to Fab, based on the concentration of BCN in the pre-reaction. All other reaction conditions were held constant across the different conjugations. In the conjugation reaction mixtures, the Fab concentration was 6 mg/mL or 12 mg/mL with 0.6 mg and 1.2 mg total Fab per reaction, respectively. All reactions were conducted in 15 v/v % DMA in 25 mM HEPES pH 7.5 buffer. The conjugation reactions were conducted at room temperature (˜25° C.) for approximately 18 h. The final average DAR and percentage of DAR0 for each conjugate was determined by SDS-PAGE densitometry and the results are shown in Table 13 (reactions P, Q, R, S).

TABLE 13

| | BCN | Fab | Avg. | |
|---|---|---|---|---|
| Reaction | equiv. | (mg/mL) | DAR | % D0 |
| J | 2 | 6 | 1.11 | 28.4 |
| K | 4 | 6 | 1.62 | 18.1 |
| L | 5 | 6 | 1.90 | 13.4 |
| M | 6 | 6 | 2.41 | 11.2 |
| N | 8 | 6 | 2.57 | 8.7 |
| O | 10 | 6 | 3.34 | 4.6 |
| P | 5 | 6 | 2.01 | 12.1 |
| Q | 6 | 6 | 2.17 | 11.8 |
| R | 7 | 6 | 2.34 | 9.0 |
| S | 6 | 12 | 2.30 | 9.3 |

### Example 11. Pre-Reaction Endo-BCN-PEG4-PFP Ester Hydrolysis

A mock pre-reaction test was used to monitor the rate of PFP ester hydrolysis by RP-UPLC. The pre-reaction was conducted using the following final reaction conditions: 1.63 mM endo-BCN-PEG4-PFP ester in 1:1 DMA to 25 mM MES pH 5.5 buffer, 20 h at 20-25 C. UPLC chromatography conditions are provided in section 5.4.3. Samples were injected on the UPLC every hour to monitor the decrease in endo-BCN-PEG4-PFP ester peak area as function of time to monitor the rate of PFP ester hydrolysis of the starting reagent. Additionally, the rate of the click reaction between the endo-BCN-PEG4-PFP ester and the oligonucleotide-PAB-VC-PEG3-azide (the oligonucleotide was a PMO) was also assessed. A pre-reaction was conducted using the following conditions: 1.63 mM endo-BCN-PEG4-PFP ester, 2.47 mM oligonucleotide-PAB-VC-PEG3-azide linker-payload (1:1.5 molar ratio) in 1:1 DMA to 25 mM MES pH 5.5 buffer, 20 h at 20-25 C. For this test, the decrease in endo-BCN-PEG4-PFP ester peak area in the reaction was monitored by RP-UPLC every 30 min, 1 h, or 5 h out to a total reaction duration of 20 h. Results are shown in FIG. 23.

### Example 12. Initial Antibody-Oligonucleotide Analyses

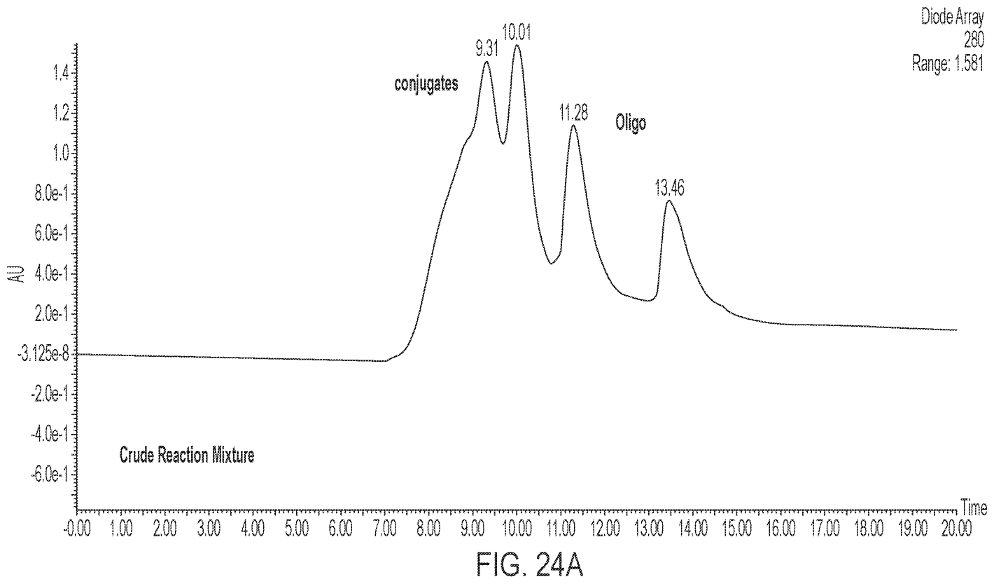

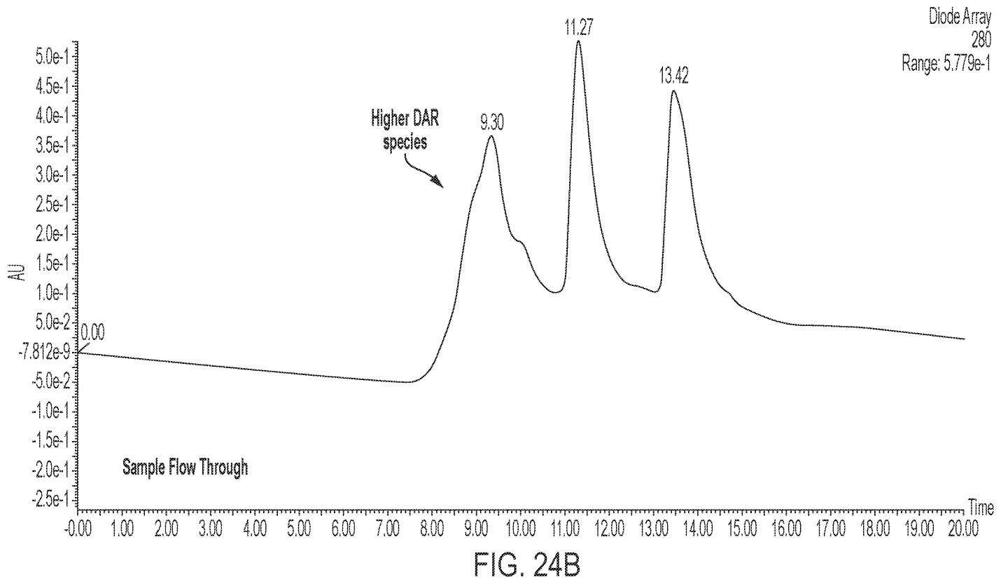

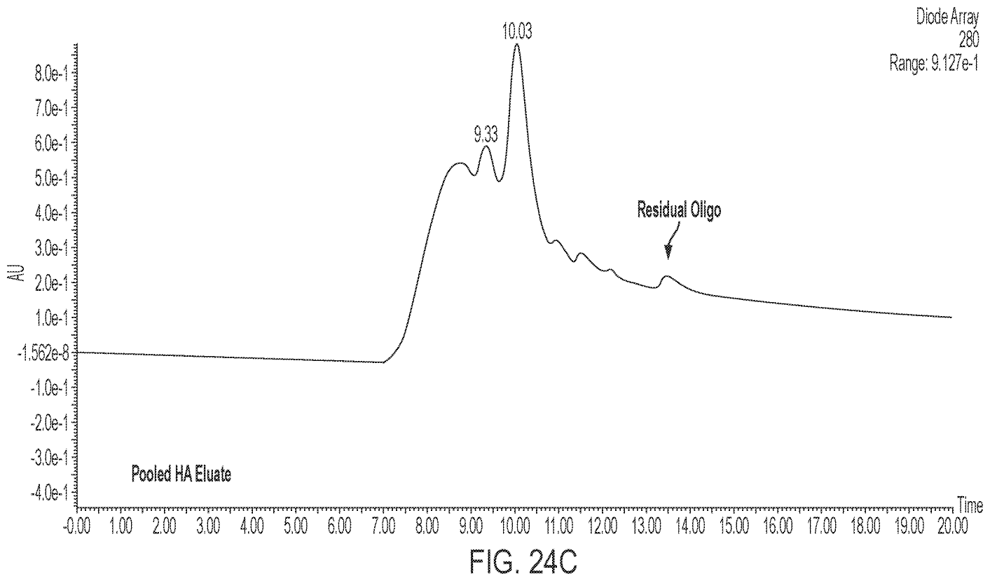

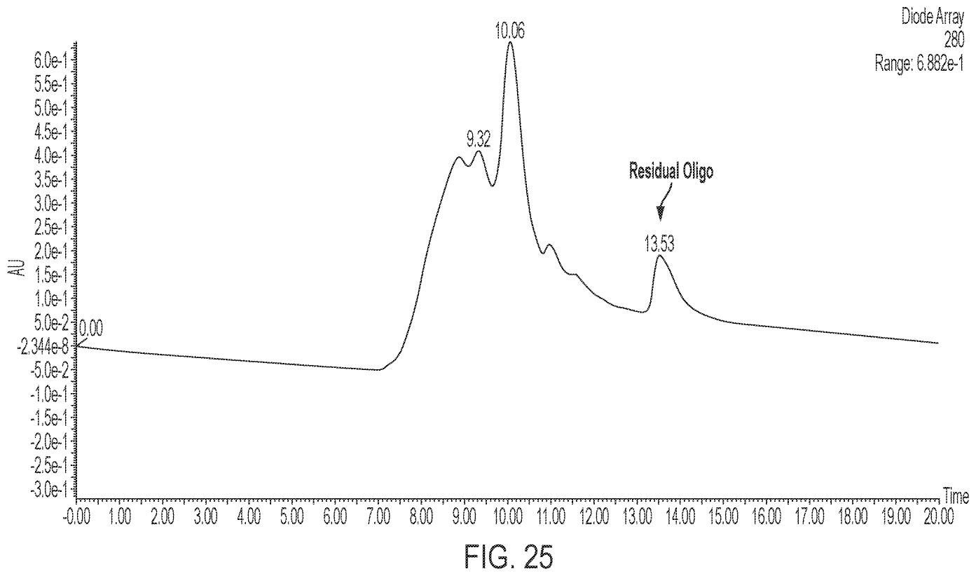

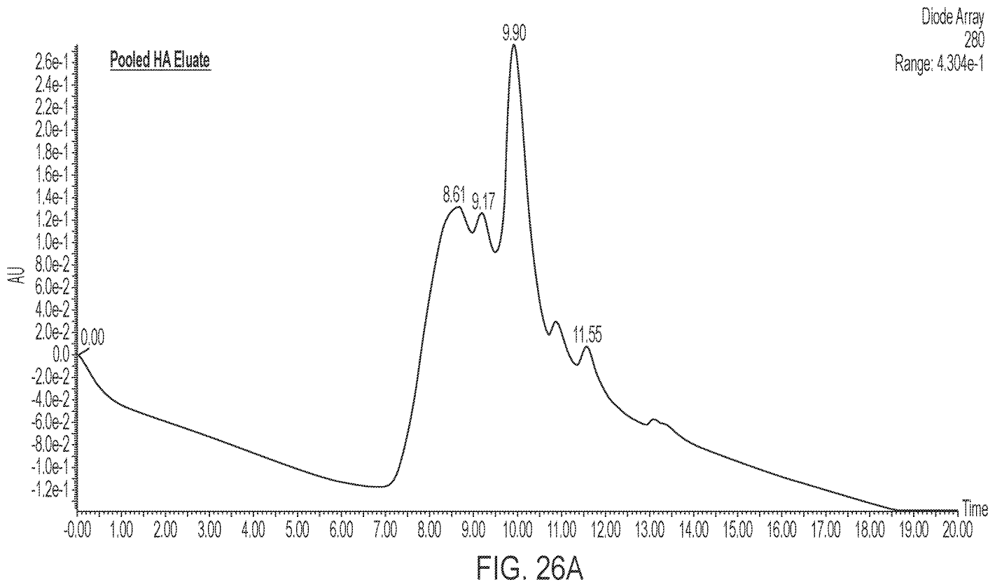

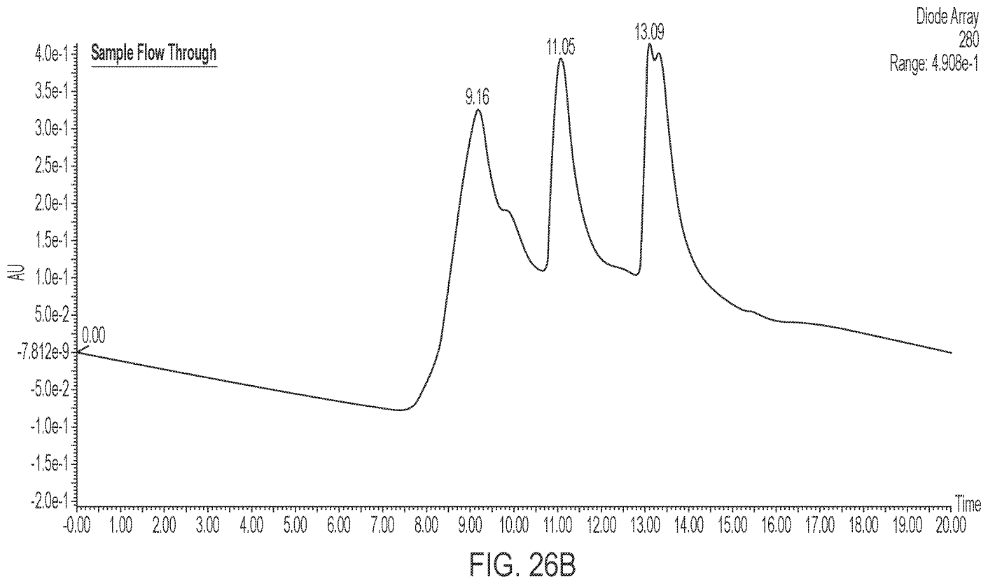

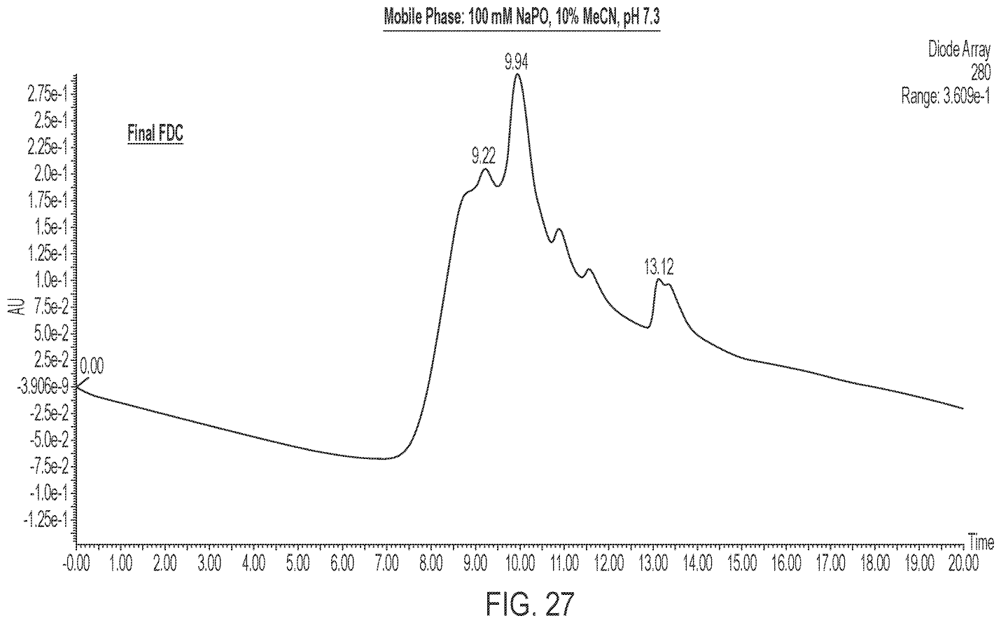

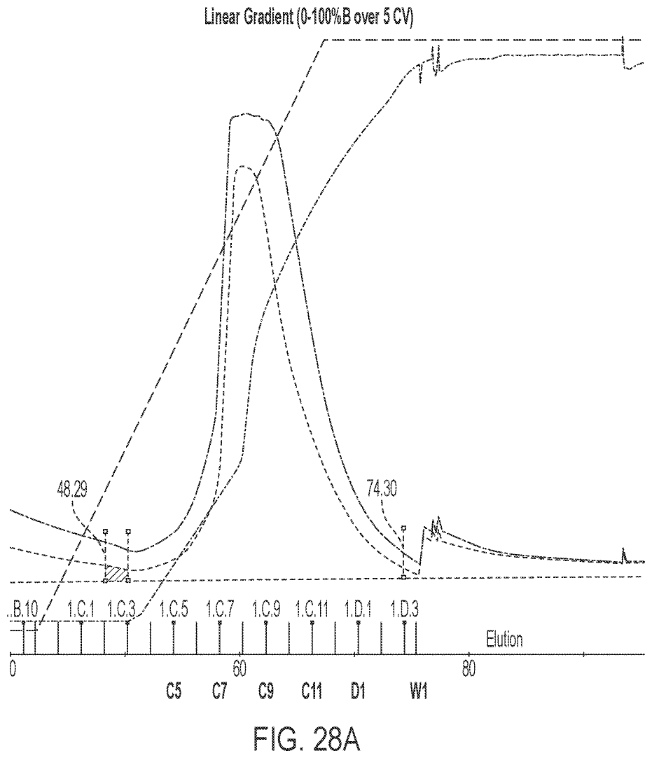

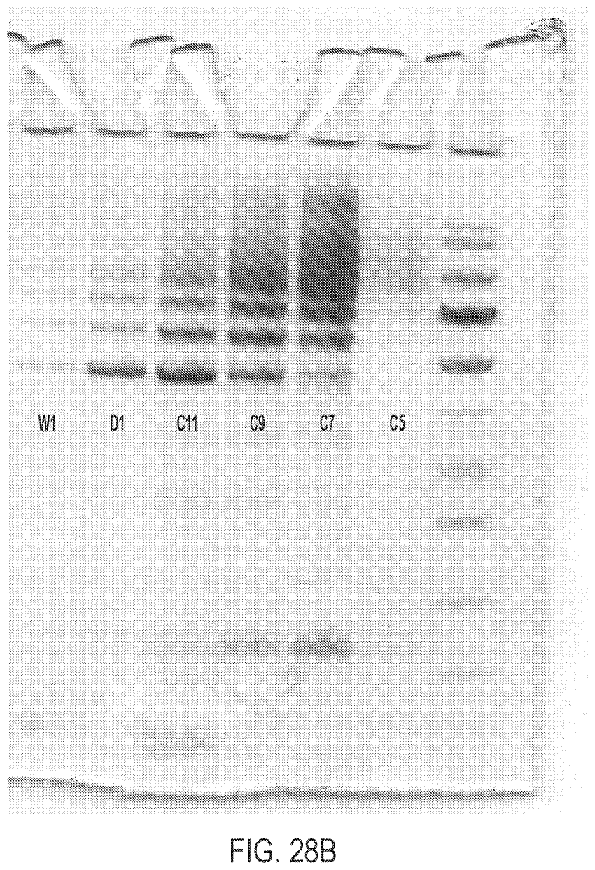

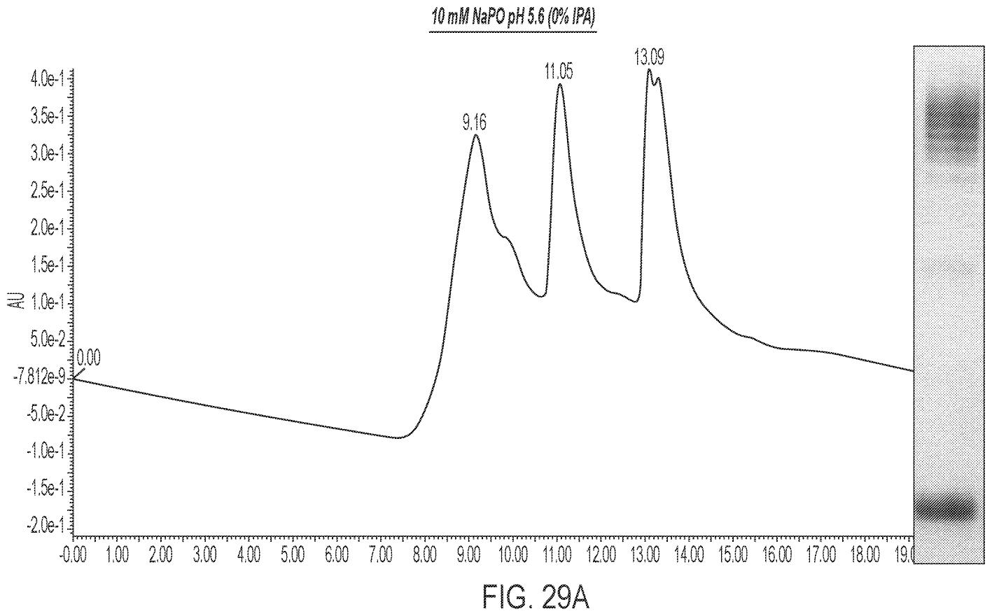

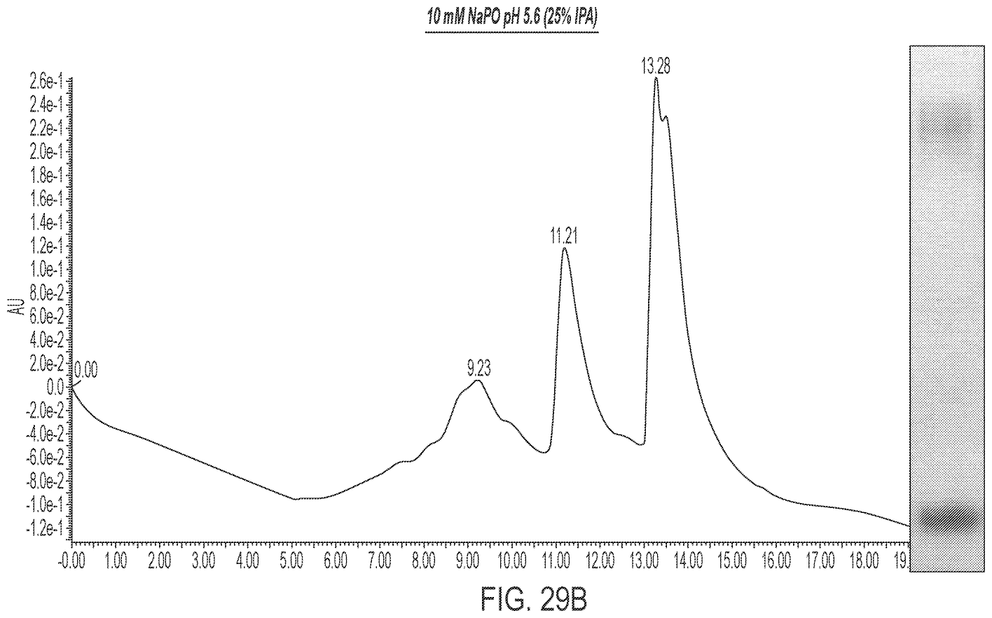

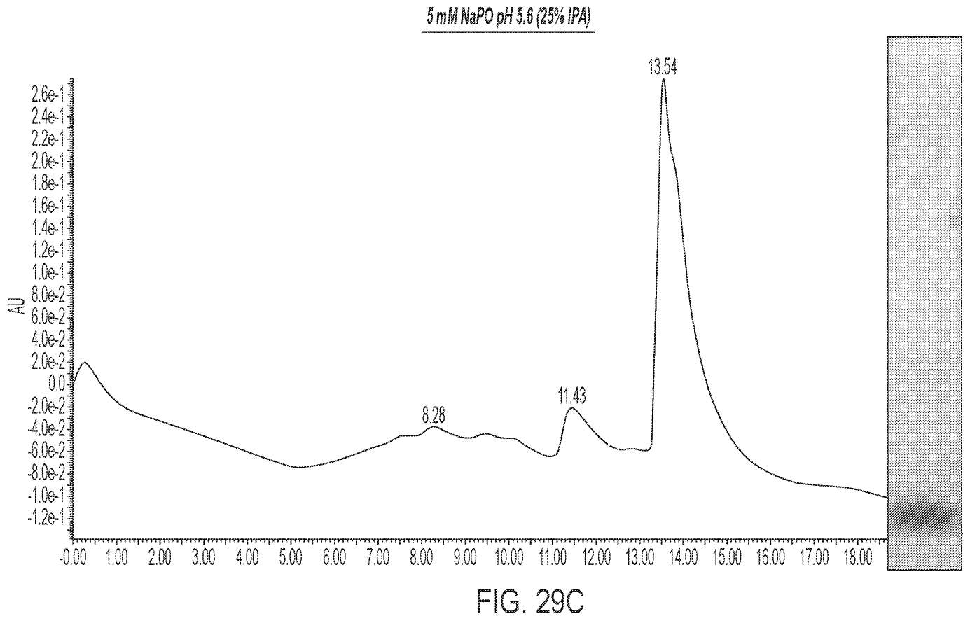

Reaction mixtures were evaluated for efficiency in producing high DAR products. Initially, a crude reaction mixture, sample flow through, and pooled HA eluate were evaluated by SEC-UPLC analysis (FIGS. 24A-24C). The crude reaction mixture contained antibody-oligonucleotide conjugates as well as unconjugated oligonucleotides (FIG. 24A). The sample flow through showed the presence of high DAR species in the presence of unconjugated oligonucleotides (FIG. 24B). Unconjugated, residual oligonucleotides were present in the pooled HA eluate (FIG. 24C). These results were consistent in the buffer exchanged final conjugate (FIG. 25), which resulted in an overall yield of 62%. Further analyses of the pooled HA eluate and sample flow through by SEC-UPLC indicated conjugate loss in a mobile phase buffer comprising 100 mM sodium phosphate and 10% MeCN at pH 7.3 (FIGS. 26A-26B). The final antibody fragment-drug conjugates (FDC) mixture in the same reaction conditions was determined to comprise unconjugated oligonucleotide (potentially oligonucleotide dimers) and a 47.2% recovery of conjugated oligonucleotides (FIG. 27). HA purification chromatogram showed low conjugate purification and the presence of unconjugated oligonucleotide (FIG. 28A-28B). Together, these results suggested a need for improved reaction conditions to yield higher concentrations of antibody-oligonucleotide conjugates.

### Example 13. Reaction Conditions

To determine conditions to yield high concentrations of antibody-oligonucleotide conjugates, reaction conditions were varied. However, increasing IPA concentration and decreasing sodium phosphate concentration appeared to improve flow through of unconjugated oligonucleotide and retention of the FDC during sample application (FIGS. 30A-30C). Finally, increasing DMA concentration improved the reaction efficiency, resulting in a higher number of antibody-oligonucleotide conjugates and a lower number of unconjugated oligonucleotides (Table 14).

TABLE 14

| | | Reaction | | |
|---|---|---|---|---|
| | % | Temperature | DAR by | |
| Reaction | DMA | (° C.) | densitometry | % D0 |
| 1 | 15 | 20 | 2.08 | 19.2 |
| 2 | 25 | 20 | 2.27 | 13.4 |
| 3 | 30 | 20 | 2.40 | 9.3 |
| 4 | 15 | 30 | 2.12 | 19.0 |
| 5 | 30 | 30 | 2.29 | 9.9 |
| 6 | 15 | 37 | 2.05 | 17.8 |
| 7 | 30 | 37 | 2.42 | 6.2 |

### Additional Embodiments

1. A mixture comprising complexes each comprising an antibody covalently linked to one or more oligonucleotides and unlinked oligonucleotides, wherein the mixture is produced by a method comprising:

(i) linking an oligonucleotide to a val-cit linker to obtain a first intermediate;

(ii) linking the first intermediate obtained in step (i) with a compound comprising a bicyclononyne to obtain a second intermediate; and

(iii) linking the second intermediate obtained in step (ii) to an antibody to obtain the complexes;

wherein the compound comprising the bicyclononyne is present in the reaction of step (iii) in an amount that is less than 5% of the starting amount of the compound in step (ii), optionally wherein the oligonucleotide is linked to the val-cit linker at the 5′ end and/or the antibody is linked via a lysine.

2. A mixture comprising complexes each comprising an antibody covalently linked to one or more oligonucleotides and unlinked oligonucleotides, wherein the mixture is produced by a method comprising:

(i) linking the oligonucleotide to a linker of Formula (A):

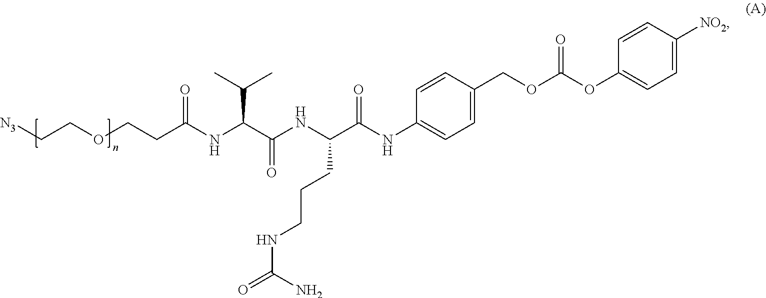

wherein n is 3; to provide an oligonucleotide of Formula (B):

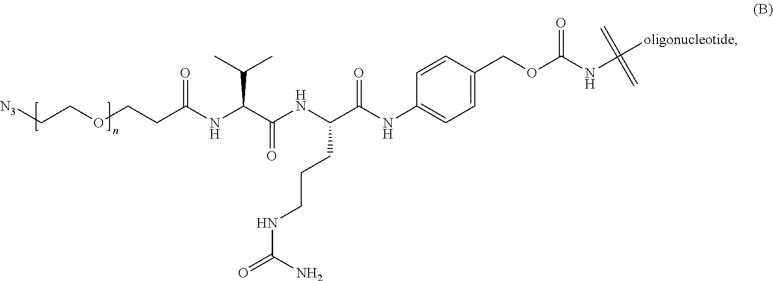

wherein n is 3;

(ii) contacting the oligonucleotide of Formula (B) with a compound of Formula (C):

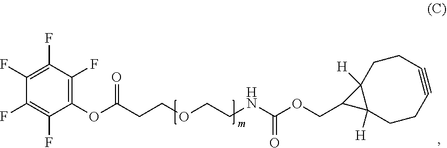

wherein m is 4; to provide an oligonucleotide of Formula (D):

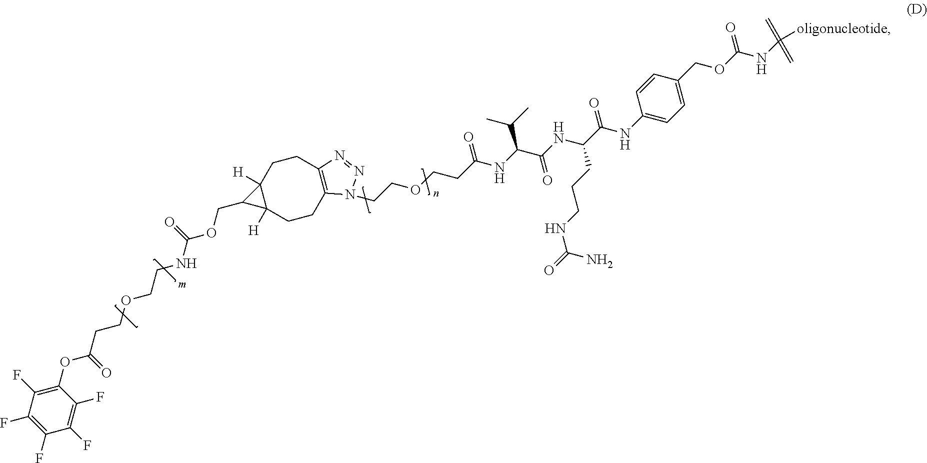

wherein n is 3 and m is 4; and

(iii) contacting the oligonucleotide of Formula (D) with an antibody to provide a complex of Formula (E):

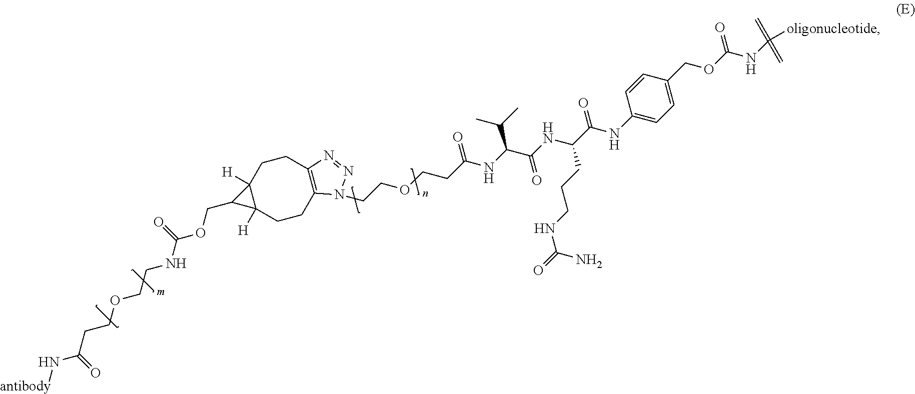

wherein n is 3 and m is 4;

wherein the compound of Formula (C) is present in the reaction mixture of step (iii) in an amount that is less than 5% of the starting amount of the compound of Formula (C) in the reaction of step (ii).

3. A method of processing complexes each comprising an antibody covalently linked to one or more oligonucleotides, the method comprising:

(i) contacting the mixture of embodiment 1 or embodiment 2 with a mixed-mode resin that comprises positively-charged metal sites and negatively charged ionic sites, under conditions in which the complexes adsorb to the mixed-mode resin, wherein the mixture comprises trace amounts of unlinked antibodies that comprise an alkyne group; and

(ii) eluting the complexes from the mixed-mode resin under conditions in which the complexes dissociate from the mixed-mode resin.

4. The method of embodiment 3, wherein the mixture in step (i) has a pH of 5.0-6.0.
5. The method of embodiment 3 or embodiment 4, wherein the mixture in step (i) has not been subjected to previous purification.
6. The method of any one of embodiments 3 to 5, wherein the mixture of step (i) comprises trace amounts of phosphate ions and/or chloride ions.
7. The method of any one of embodiments 3 to 5, wherein the mixture of step (i) does not comprise phosphate ions and/or chloride ions.
8. The method of any one of embodiments 3 to 5, wherein the mixed-mode resin is an apatite resin.
9. The method of embodiment 8, wherein the apatite resin is a hydroxyapatite resin, a ceramic hydroxyapatite resin, a hydroxyfluoroapatite resin, a fluoroapatite resin, or a chlorapatite resin.
10. The method of any one of embodiments 3 to 9, wherein the unlinked oligonucleotide does not adsorb to the mixed-mode resin in step (i).
11. The method of any one of embodiments 3 to 9, wherein some or all of the unlinked oligonucleotides adsorb to the mixed-mode resin in step (i).
12. The method of embodiment 11, further comprising washing the mixed-mode resin between step (i) and step (ii) with a washing solution comprising up to 20 mM phosphate ions and/or up to 30 mM chloride ions, optionally wherein the solution comprises up to 10 mM phosphate ions and/or up to 25 mM chloride ions.
13. The method of embodiment 12, wherein the washing solution has a pH of 5.0-7.6.
14. The method of embodiment 12 or embodiment 13, wherein most or all of the unlinked oligonucleotides are removed from the mixed-mode resin in the washing step.
15. The method of any one of embodiments 3 to 14, wherein step (ii) comprises applying an elution solution comprising at least 30 mM phosphate ions and/or at least 50 mM chloride ions to the mixed-mode resin to elute the complexes, optionally wherein the elution solution comprises at least 100 mM phosphate ions and/or at least 100 mM chloride ions.
16. The method of embodiment 15, wherein the elution solution has a pH of 7.5-8.5.
17. The method of any one of embodiments 3 to 16, wherein the antibody is a full length IgG, a Fab fragment, a Fab′ fragment, a F(ab′)2 fragment, a scFv, or a Fv fragment.
18. The method of any one of embodiments 3 to 17, wherein the antibody is an anti-transferrin receptor antibody.
19. The method of any one of embodiments 3 to 18, wherein the oligonucleotide is single stranded.
20. The method of embodiment 19, wherein the oligonucleotide is an antisense oligonucleotide, optionally a gapmer.
21. The method of embodiment 20, wherein the oligonucleotide is one strand of a double stranded oligonucleotide, optionally wherein the double stranded oligonucleotide is an siRNA, and optionally wherein the one strand is the sense strand of the siRNA.
22. The method of any one of embodiments 3 to 21, wherein the oligonucleotide comprises at least one modified internucleotide linkage, optionally wherein the at least one modified internucleotide linkage is a phosphorothioate linkage.
23. The method of any one of embodiments 3 to 22, wherein the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.
24. The method of any one of embodiments 3 to 23, wherein the oligonucleotide is 10-50 nucleotides in length, optionally 15-25 nucleotides in length.
25. The method of any one of embodiments 3 to 24, wherein the antibody is covalently linked to the 5′ of the oligonucleotide.
26. The method of any one of embodiments 3 to 25, wherein the antibody is covalently linked to the 3′ of the oligonucleotide.
27. The method of any one of embodiments 3 to 26, wherein the antibody is linked via a lysine.
28. The method of any one of embodiments 2 to 27, wherein the eluent obtained from step (ii) comprises undetectable levels of unlinked oligonucleotide.
29. A method of processing complexes each comprising an antibody covalently linked to one or more charge-neutral oligonucleotides, the method comprising:

(i) contacting a mixture comprising an organic solvent, the complexes and unlinked charge-neutral oligonucleotides with a mixed-mode resin that comprises positively-charged metal sites and negatively charged ionic sites, under conditions in which the complexes adsorb to the mixed-mode resin, and

(ii) eluting the complexes from the mixed-mode resin under conditions in which the complexes dissociate from the mixed-mode resin.

30. The method of embodiment 29, wherein the mixed-mode resin is an apatite resin.
31. The method of embodiment 2, wherein the apatite resin is a hydroxyapatite resin, a ceramic hydroxyapatite resin, a hydroxyfluoroapatite resin, a fluoroapatite resin, or a chlorapatite resin.
32. The method of any one of embodiments 29 to 31, wherein the organic solvent is Dimethylacetamide (DMA), isopropyl alcohol (IPA), dimethyl sulfoxide (DMSO), acetonitrile (ACN), or propylene glycol (PG).
33. The method of any one of embodiments 29 to 32, wherein the organic solvent is at 5%-20% (v/v) in the mixture in step (i), optionally wherein the organic solvent is at 15% (v/v) in the mixture in step (i).
34. The method of any one of embodiments 29 to 33, wherein the mixture in step (i) does not comprise a phosphate ion or a chloride ion.
35. The method of any one of embodiments 29 to 34, wherein the mixture in step (i) further comprises up to 10 mM phosphate ions and/or up to 20 mM chloride ions.
36. The method of any one of embodiments 29 to 35, wherein the mixture in step (i) has a pH of 5.0-6.0.
37. The method of any one of embodiments 29 to 36, wherein the unlinked charge-neutral oligonucleotide does not adsorb to the mixed-mode resin in step (i).
38. The method of any one of embodiments 29 to 37, further comprising washing the mixed-mode resin between step (i) and step (ii) with a washing solution comprising of an organic solvent, optionally wherein the organic solvent is Dimethylacetamide (DMA), isopropyl alcohol (IPA), dimethyl sulfoxide (DMSO), acetonitrile (ACN), or propylene glycol (PG).
39. The method of embodiment 38, wherein the organic solvent is at 5%-20% (v/v) in the washing solution, optionally wherein the organic solvent is at 15% (v/v) in the washing solution.
40. The method of embodiment 38 or embodiment 39, wherein the washing solution further comprises up to 10 mM phosphate ions and/or up to 20 mM chloride ions.
41. The method of any one of embodiments 29 to 40, wherein step (ii) comprises applying an elution solution to the mixed-mode resin to elute the complexes, wherein the elution solution comprises an organic solvent, optionally wherein the organic solvent is Dimethylacetamide (DMA), isopropyl alcohol (IPA), dimethyl sulfoxide (DMSO), acetonitrile (ACN), or propylene glycol (PG).
42. The method of embodiment 41, wherein the organic solvent is at 10%-20% (v/v) in the elution solution, optionally wherein the organic solvent is at 10% (v/v) in the elution solution.
43. The method of embodiment 41 or embodiment 42, wherein the elution solution comprises at least 30 mM phosphate ions, optionally wherein the elution solution comprises at least 100 mM phosphate ions.
44. The method of embodiment 43, wherein the elution solution does not comprise chloride ions.
45. The method of embodiment 41 or embodiment 42, wherein the elution solution comprises a gradually increasing concentration of phosphate ions, optionally wherein the concentration of the phosphate ions increases from at least 10 mM to at least 100 mM.
46. The method of any one of embodiments 41 to 45, wherein the elution solution has a pH of 7.6-8.5.
47. The method of any one of embodiments 29 to 46, wherein the antibody is a full-length IgG, a Fab fragment, a Fab′ fragment, a F(ab′)2 fragment, a scFv, or a Fv fragment.
48. The method of any one of embodiments 29 to 47, wherein the antibody is an anti-transferrin receptor antibody.
49. The method of any one of embodiments 29 to 48, wherein the charge-neutral oligonucleotide is single stranded.
50. The method of embodiment 49, wherein the charge-neutral oligonucleotide is an antisense oligonucleotide.
51. The method of any one of embodiments 29 to 50, wherein the charge-neutral oligonucleotide is a phosphorodiamidate morpholino oligomer (PMO).
52. The method of any one of embodiments 29 to 51, wherein the charge-neutral oligonucleotide is 10-50 nucleotides in length, optionally 20-30 nucleotides in length.
53. The method of any one of embodiments 29 to 52, wherein the antibody is covalently linked to the 5′ of the charge-neutral oligonucleotide.
54. The method of any one of embodiments 29 to 53, wherein the antibody is covalently linked to the 3′ of the charge-neutral oligonucleotide.
55. The method of any one of embodiments 29 to 54, wherein the antibody is covalently linked to the charge-neutral oligonucleotide via a linker, optionally a Val-cit linker.
56. The method of embodiment 55, wherein the linker comprises a structure of:

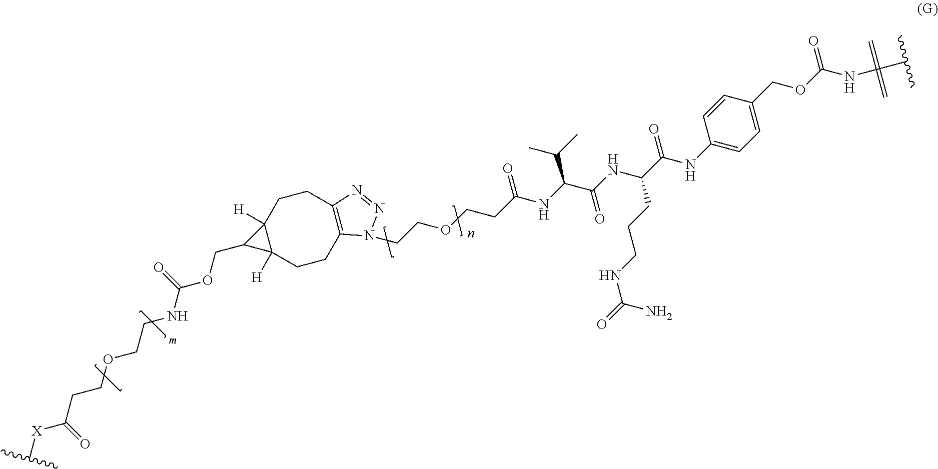

wherein n is 3 and m is 4.
57. The method of embodiment 56, wherein the complex comprises a structure of:

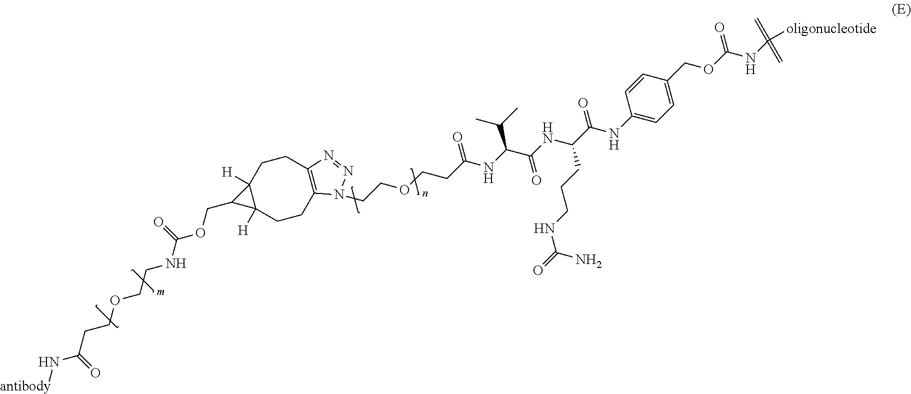

wherein n is 3 and m is 4, and wherein the antibody is linked via a lysine.
58. The method of any one of embodiments 29 to 57, wherein the complexes in the mixture of step (i) have an average drug to antibody ratio (DAR) of at least about 1.8.
59. The method of any one of embodiments 29 to 58, wherein the eluent obtained from step (ii) comprises undetectable levels of unlinked charge-neutral oligonucleotides.
60. The method of any one of embodiments 29 to 59, wherein the complex is produced via linking a charge-neutral oligonucleotide to an antibody, wherein the charge-neutral oligonucleotide comprises a structure of:

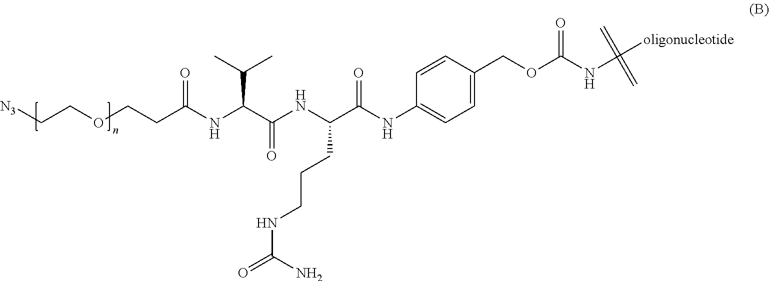

and wherein the antibody comprises a structure of:

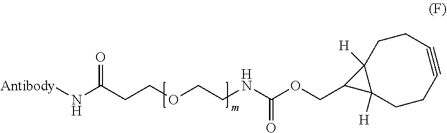

wherein n is 3 and m is 4.
61. The method of any one of embodiments 29 to 59, wherein the complex is produced via linking a charge-neutral oligonucleotide to an antibody, wherein the charge-neutral oligonucleotide comprises a structure of:

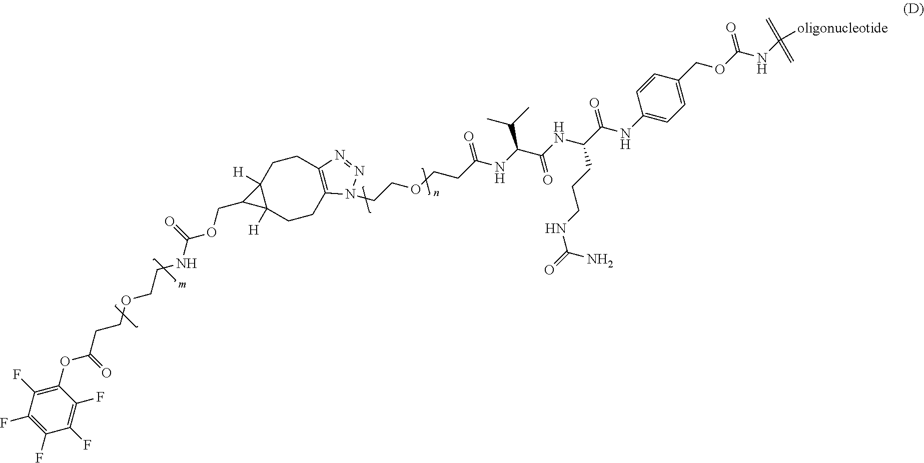

wherein n is 3 and m is 4.
62. A method of producing a complex comprising an antibody covalently linked to one or more oligonucleotides, the method comprising:

(i) obtaining an oligonucleotide comprising a structure of:

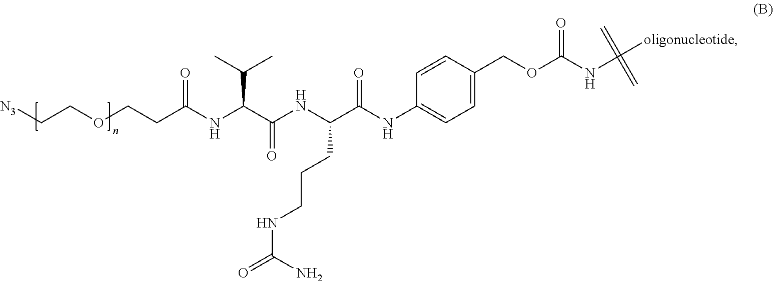

wherein n is 3;

(ii) obtaining an antibody comprises a structure of:

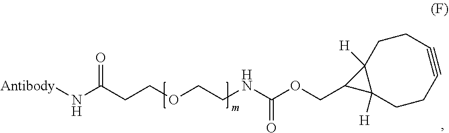

wherein m is 4; and

(iii) reacting the oligonucleotide in step (i) and the antibody obtained in step (ii) to obtain the complex.

63. A method of producing a complex comprising an antibody covalently linked to one or more oligonucleotides, the method comprising:

(i) obtaining an oligonucleotide comprising a structure of:

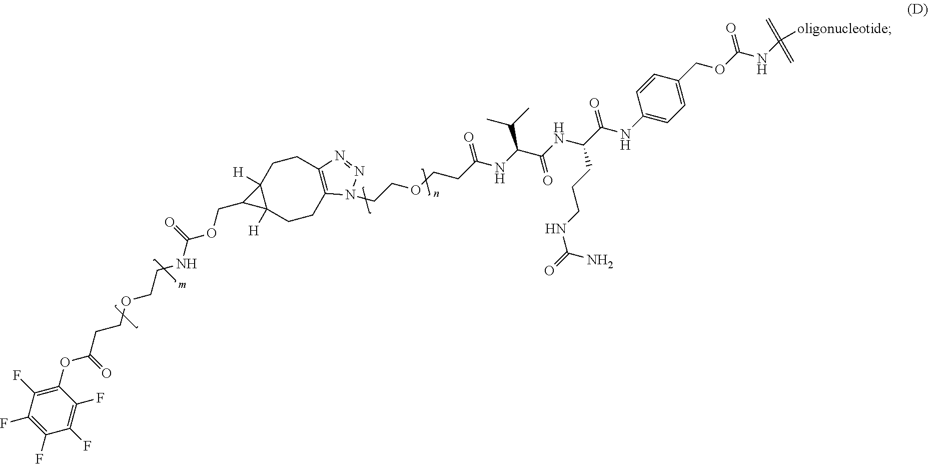

wherein n is 3 and wherein m is 4;

(ii) obtaining an antibody; and

(iii) reacting the oligonucleotide in step (i) and the antibody obtained in step (ii) to obtain the complex.

64. The method of embodiment 62 or embodiment 63, wherein the complex comprises a structure of:

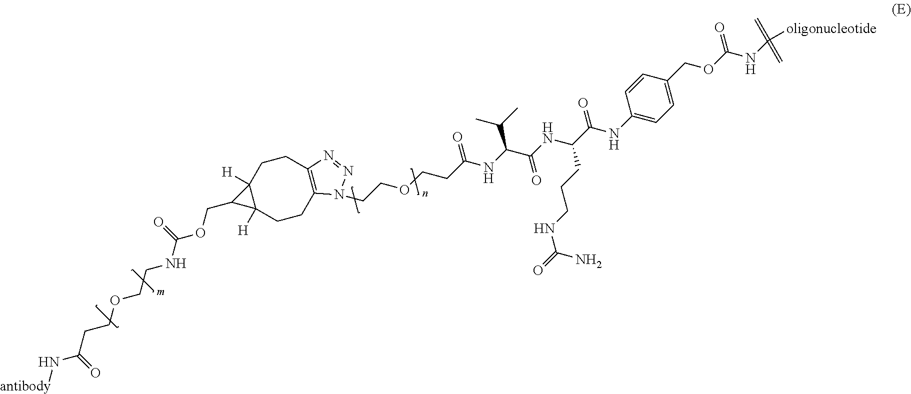

wherein n is 3 and m is 4, and wherein the antibody is linked via a lysine.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A method of processing complexes each comprising an antibody covalently linked to one or more charge-neutral oligonucleotides, the method comprising:
(i) contacting a mixture comprising an organic solvent, the complexes and unlinked charge-neutral oligonucleotides with a mixed-mode resin that comprises positively-charged metal sites and negatively charged ionic sites, under conditions in which the complexes adsorb to the mixed-mode resin, and
(ii) eluting the complexes from the mixed-mode resin under conditions in which the complexes dissociate from the mixed-mode resin.

2. The method of any one of claim 1, wherein the organic solvent is Dimethylacetamide (DMA), isopropyl alcohol (IPA), dimethyl sulfoxide (DMSO), acetonitrile (ACN), or propylene glycol (PG).

3. The method of claim 1 or 2, wherein the organic solvent is at 5%-30% (v/v) in the mixture in step (i), optionally wherein the organic solvent is at 15% (v/v) in the mixture in step (i).

4. The method of any one of claims 1-3, wherein the mixture in step (i) further comprises up to 10 mM phosphate ions and/or up to 20 mM chloride ions.

5. The method of any one of claims 1-4, further comprising washing the mixed-mode resin between step (i) and step (ii) with a washing solution comprising an organic solvent, optionally wherein the organic solvent is Dimethylacetamide (DMA), isopropyl alcohol (IPA), dimethyl sulfoxide (DMSO), acetonitrile (ACN), or propylene glycol (PG).

6. The method of claim 5, wherein the organic solvent is at 5%-30% (v/v) in the washing solution, optionally wherein the organic solvent is at 15% (v/v) in the washing solution.

7. The method of claim 5 or claim 6, wherein the washing solution further comprises up to 10 mM phosphate ions and/or up to 20 mM chloride ions.

8. The method of any one of claims 1-7, wherein step (ii) comprises applying an elution solution to the mixed-mode resin to elute the complexes, wherein the elution solution comprises an organic solvent, optionally wherein the organic solvent is Dimethylacetamide (DMA), isopropyl alcohol (IPA), dimethyl sulfoxide (DMSO), acetonitrile (ACN), or propylene glycol (PG).

9. The method of claim 8, wherein the organic solvent is at 10%-30% (v/v) in the elution solution, optionally wherein the organic solvent is at 10% (v/v) in the elution solution.

10. The method of claim 8 or claim 9, wherein the elution solution comprises at least 30 mM phosphate ions, optionally wherein the elution solution comprises at least 100 mM phosphate ions.

11. The method of claim 8 or claim 9, wherein the elution solution comprises a gradually increasing concentration of phosphate ions, optionally wherein the concentration of the phosphate ions increases from at least 10 mM to at least 100 mM.

12. The method of any one of claims 8-11, wherein the elution solution has a pH of 7.6-8.5.

13. A method of producing a complex comprising an antibody covalently linked to one or more oligonucleotides, the method comprising:
(i) obtaining an oligonucleotide comprising a structure of: 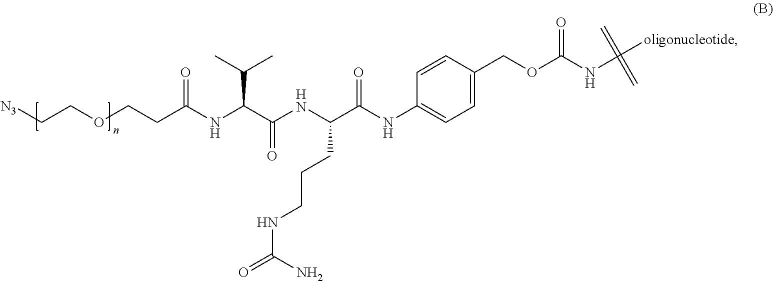
wherein n is 3;
(ii) obtaining an antibody comprises a structure of: 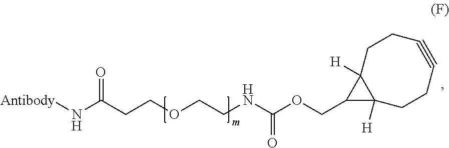
wherein m is 4; and
(iii) reacting the oligonucleotide in step (i) and the antibody obtained in step (ii) to obtain the complex.

14. A method of producing a complex comprising an antibody covalently linked to one or more oligonucleotides, the method comprising:
(i) obtaining an oligonucleotide comprising a structure of: 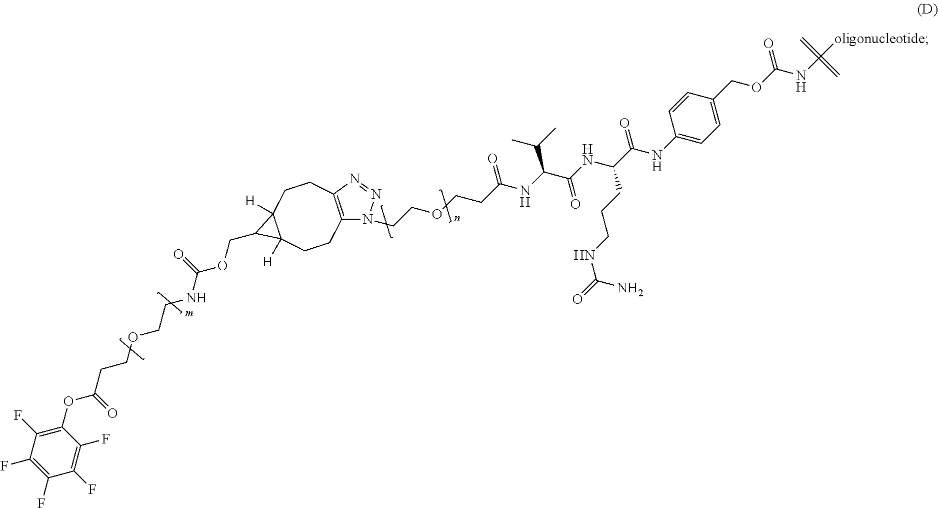
wherein n is 3 and wherein m is 4;
(ii) obtaining an antibody; and
(iii) reacting the oligonucleotide in step (i) and the antibody obtained in step (ii) to obtain the complex.

15. The method of claim 14, wherein the complex comprises a structure of: 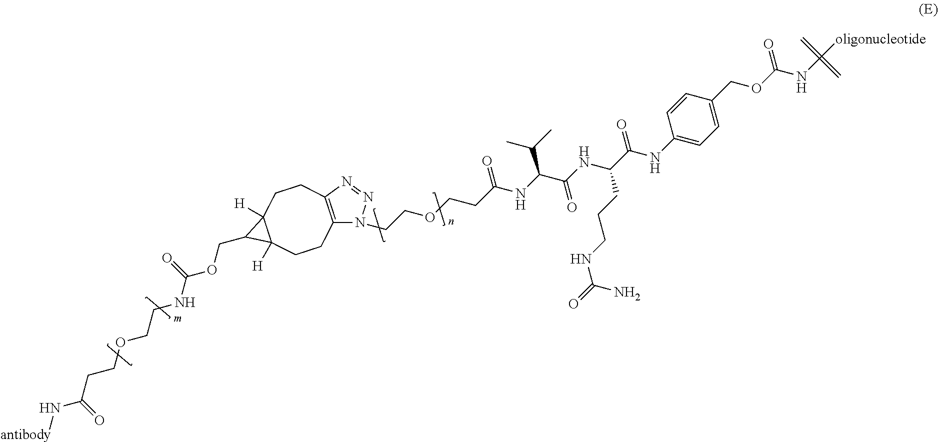
wherein n is 3 and m is 4, and wherein the antibody is linked via a lysine.

16. A mixture comprising complexes, each complex comprising an antibody covalently linked to one or more oligonucleotides, and unlinked oligonucleotides, wherein the mixture is produced by a method comprising:
(i) obtaining a first intermediate comprising an oligonucleotide covalently linked to a cleavable linker comprising a valine-citrulline sequence;
(ii) linking the first intermediate obtained in step (i) with a compound comprising a bicyclononyne to obtain a second intermediate; and
(iii) linking the second intermediate obtained in step (ii) to an antibody to obtain the complexes;
wherein the compound comprising the bicyclononyne is present in the reaction of step (iii) in an amount that is less than 5% of the starting amount of the compound in step (ii), optionally wherein the oligonucleotide is covalently linked to the cleavable linker comprising the valine-citrulline sequence at the 5′ end and/or the antibody is linked via a lysine.

17. A mixture comprising complexes, each complex comprising an antibody covalently linked to one or more oligonucleotides, and ii) unlinked oligonucleotides, wherein the mixture is produced by a method comprising:
(i) combining one or more oligonucleotides with a linker of Formula (A): 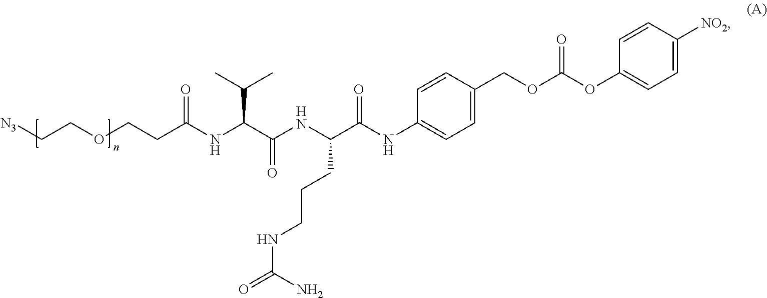
wherein n is 3; under reaction conditions that produce product of Formula (B): 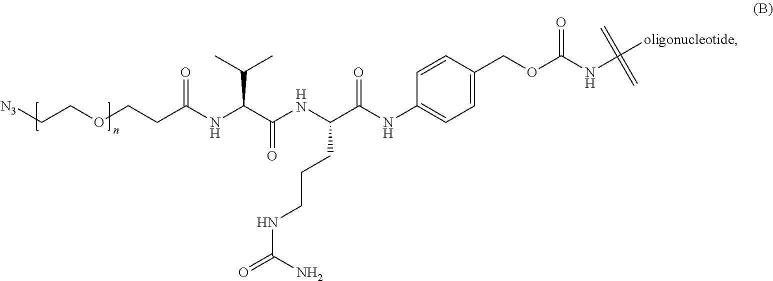
wherein n is 3;
(ii) contacting the product of Formula (B) with a compound of Formula (C): 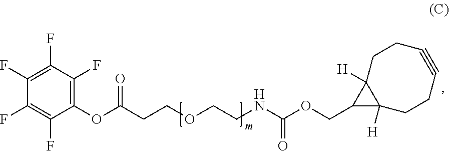
wherein m is 4; under reaction conditions that produce a product of Formula (D): 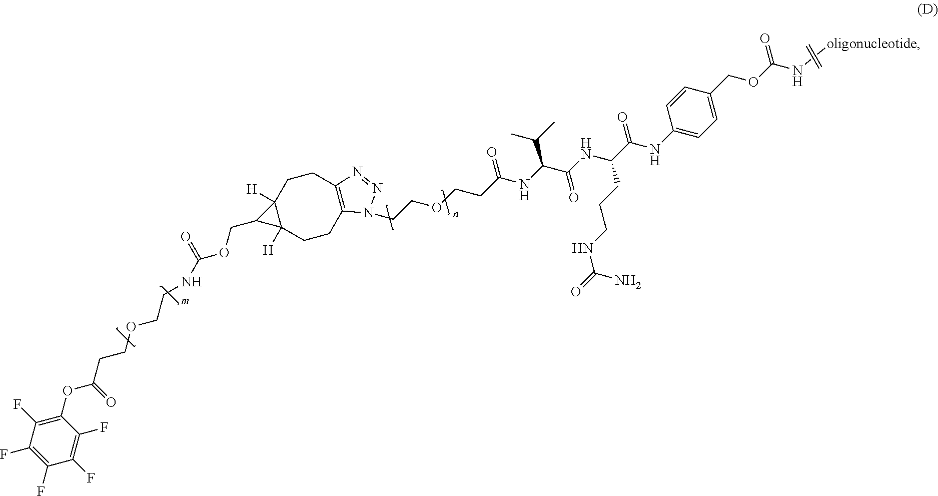
wherein n is 3 and m is 4; and
(iii) contacting the product of Formula (D) with an antibody under reaction conditions that produce a complex of Formula (E): 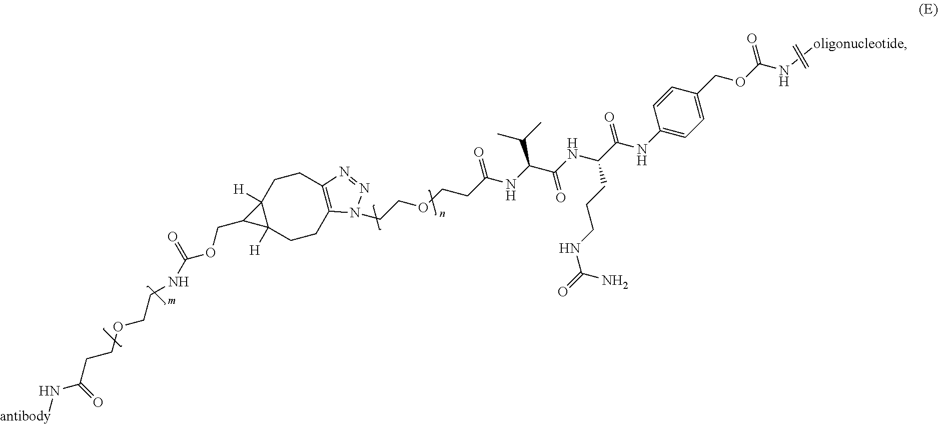
wherein n is 3 and m is 4;
wherein the compound of Formula (C) is present in the reaction of step (iii) in an amount that is less than 5% of the starting amount of the compound of Formula (C) in the reaction of step (ii).

18. A method of processing complexes each comprising an antibody covalently linked to one or more oligonucleotides, the method comprising:
(i) contacting the mixture of claim 16 or claim 17 with a mixed-mode resin that comprises positively-charged metal sites and negatively charged ionic sites, under conditions in which the complexes adsorb to the mixed-mode resin, wherein the mixture comprises trace amounts of unlinked antibodies that comprise an alkyne group; and
(ii) eluting the complexes from the mixed-mode resin under conditions in which the complexes dissociate from the mixed-mode resin.

19. The method of claim 18, wherein the mixture in step (i) has not been subjected to previous purification.

20. The method of 18 or 19, wherein the mixture of step (i) comprises trace amounts of phosphate ions and/or chloride ions.

21. The method of claim 18, further comprising washing the mixed-mode resin between step (i) and step (ii) with a washing solution comprising up to 20 mM phosphate ions and/or up to 30 mM chloride ions, optionally wherein the solution comprises up to 10 mM phosphate ions and/or up to 25 mM chloride ions.

22. The method of claim 21, wherein the washing solution has a pH of 5.0-7.6.

23. The method of claim 21 or claim 22, wherein most or all unlinked oligonucleotides are removed from the mixed-mode resin in the washing step.

24. The method of any one of claims 18-23, wherein step (ii) comprises applying an elution solution comprising at least 30 mM phosphate ions and/or at least 50 mM chloride ions to the mixed-mode resin to elute the complexes, optionally wherein the elution solution comprises at least 100 mM phosphate ions and/or at least 100 mM chloride ions.

25. The method of claim 24, wherein the elution solution has a pH of 7.5-8.5.

26. The method of any one of claims 18-25, wherein the antibody is an anti-transferrin receptor antibody.

27. The method of any one of claims 18-26, wherein the oligonucleotide is a charged oligonucleotide.

28. The method of claim 27, wherein the oligonucleotide is a negatively charged oligonucleotide.

29. The method of claim 27 or claim 28, wherein the oligonucleotide is single-stranded.

30. The method of any one of claims 27-29, wherein the oligonucleotide is an antisense oligonucleotide, optionally a gapmer.

31. The method of claim 30, wherein the oligonucleotide is one strand of a double stranded oligonucleotide, optionally wherein the double stranded oligonucleotide is an siRNA, and optionally wherein the one strand is the sense strand of the siRNA.

32. The method of any one of claims 18-31, wherein the oligonucleotide comprises at least one modified internucleotide linkage, optionally wherein the at least one modified internucleotide linkage is a phosphorothioate linkage.

33. The method of any one of claims 18-32, wherein the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.


SEQUENCE LISTING (45 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 3 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 4 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 5 - protein, 197 aa, Artificial Sequence
  Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 6 - protein, 7 aa, Artificial Sequence
  Synthetic
ASSLNIA

SEQ ID NO: 7 - protein, 12 aa, Artificial Sequence
  Synthetic
SKTFNTHPQSTP

SEQ ID NO: 8 - protein, 12 aa, Artificial Sequence
  Synthetic
TARGEHKEEELI

SEQ ID NO: 9 - protein, 9 aa, Artificial Sequence
  Synthetic
CQAQGQLVC

SEQ ID NO: 10 - protein, 9 aa, Artificial Sequence
  Synthetic
CSERSMNFC

SEQ ID NO: 11 - protein, 9 aa, Artificial Sequence
  Synthetic
CPKTRRVPC

SEQ ID NO: 12 - protein, 20 aa, Artificial Sequence
  Synthetic
WLSEAGPVVTVRALRGTGSW

SEQ ID NO: 13 - protein, 9 aa, Artificial Sequence
  Synthetic
CMQHSMRVC

SEQ ID NO: 14 - protein, 7 aa, Artificial Sequence
  Synthetic
DDTRHWG

SEQ ID NO: 15-16 - skipped (2 entries)

SEQ ID NO: 17 - protein, 5 aa, Artificial Sequence
  Synthetic
SYWMH

SEQ ID NO: 18 - protein, 17 aa, Artificial Sequence
  Synthetic
EINPTNGRTNYIEKFKS

SEQ ID NO: 19 - protein, 7 aa, Artificial Sequence
  Synthetic
GTRAYHY

SEQ ID NO: 20 - protein, 11 aa, Artificial Sequence
  Synthetic
RASDNLYSNLA

SEQ ID NO: 21 - protein, 7 aa, Artificial Sequence
  Synthetic
DATNLAD

SEQ ID NO: 22 - protein, 9 aa, Artificial Sequence
  Synthetic
QHFWGTPLT

SEQ ID NO: 23 - protein, 7 aa, Artificial Sequence
  Synthetic
GYTFTSY

SEQ ID NO: 24 - protein, 6 aa, Artificial Sequence
  Synthetic
NPTNGR

SEQ ID NO: 25 - protein, 6 aa, Artificial Sequence
  Synthetic
TSYWMH

SEQ ID NO: 26 - protein, 13 aa, Artificial Sequence
  Synthetic
WIGEINPTNGRTN

SEQ ID NO: 27 - protein, 6 aa, Artificial Sequence
  Synthetic
ARGTRA

SEQ ID NO: 28 - protein, 7 aa, Artificial Sequence
  Synthetic
YSNLAWY

SEQ ID NO: 29 - protein, 10 aa, Artificial Sequence
  Synthetic
LLVYDATNLA

SEQ ID NO: 30 - protein, 8 aa, Artificial Sequence
  Synthetic
QHFWGTPL

SEQ ID NO: 31 - protein, 9 aa, Artificial Sequence
  Synthetic
QHFAGTPLT

SEQ ID NO: 32 - protein, 8 aa, Artificial Sequence
  Synthetic
QHFAGTPL

SEQ ID NO: 33 - protein, 116 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 34 - protein, 107 aa, Artificial Sequence
  Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 35 - protein, 116 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 36 - protein, 107 aa, Artificial Sequence
  Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 37 - protein, 330 aa, Homo sapiens [sequence omitted]

SEQ ID NO: 38 - protein, 107 aa, Artificial Sequence [sequence omitted]

SEQ ID NO: 39 - protein, 446 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 446 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial Sequence
  Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 226 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 44 - protein, 226 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 45 - protein, 13 aa, Artificial Sequence
  Synthetic
CPAPELLGGPSVF